(19) United States
(12) Patent Application Publication
(10) Pub. No.: US 2019/0322741 A1
(43) Pub. Date: Oct. 24, 2019

(54) Methods for the Use of CD32B x CD79B-Binding Molecules in the Treatment of Inflammatory Diseases and Disorders

(71) Applicant: MacroGenics, Inc., Rockville, MD (US)

(72) Inventors: Wei Chen, Gaithersburg, MD (US); Paul A. Moore, North Potomac, MD (US); Naimish Bharat Pandya, Clarksville, MD (US); Ezio Bonvini, Potomac, MD (US); Jon Marc Wigginton, Rockville, MD (US)

(73) Assignee: MacroGenics, Inc., Rockville, MD (US)

(21) Appl. No.: 16/307,385

(22) PCT Filed: Jun. 6, 2017
(86) PCT No.: PCT/US2017/036079

§ 371 Date: Dec. 5, 2018

Related U.S. Application Data
(60) Provisional application No. 62/432,328, filed on Dec. 9, 2016.
(60) Provisional application No. 62/346,717, filed on Jun. 7, 2016.

(51) Int. Cl. C07K 16/28; C07K 16/46; A61P 37/06

(52) U.S. Cl. CPC C07K 16/283; C07K 16/46; A61P 37/06; C07K 2317/73; C07K 2317/52; C07K 2317/626; C07K 2317/31

(57) ABSTRACT

The present invention is directed to methods for using bispecific binding molecules that possess a binding site specific for an epitope of CD32B and a binding site specific for an epitope of CD79B, and are thus capable of simultaneous binding to CD32B and CD79B. The invention particularly concerns such molecules that are bispecific antibodies or bispecific diabodies (and especially such diabodies that additionally comprise an Fc Domain). The invention is directed to the use of such molecules, and to the use of pharmaceutical compositions that contain such molecules in the treatment of inflammatory diseases or conditions.

### CROSS-REFERENCE TO RELATED APPLICATIONS

This application is a § 371 Application of PCT/US2017/036079 (filed Jun. 6, 2017, pending), which application claims priority to U.S. Patent Applications Ser. Nos. 62/346,717 (filed on Jun. 7, 2016; now lapsed) and 62/432,328 (filed on Dec. 9, 2016; now lapsed), each of which applications is herein incorporated by reference in its entirety.

### REFERENCE TO SEQUENCE LISTING

This application includes one or more Sequence Listings pursuant to 37 C.F.R. 1.821 et seq., which are disclosed in computer-readable media (file name: 1301_0145PCT_ST25.txt, created on May 19, 2017, and having a size of 59,748 bytes), which file is herein incorporated by reference in its entirety.

### BACKGROUND OF THE INVENTION

### Field of the Invention

The present invention is directed to methods for using bispecific binding molecules that possess a binding site specific for an epitope of CD32B and a binding site specific for an epitope of CD79B, and are thus capable of simultaneous binding to CD32B and CD79B. The invention particularly concerns such molecules that are bispecific antibodies or bispecific diabodies (and especially such diabodies that additionally comprise an Fc Domain). The invention is directed to the use of such molecules, and to the use of pharmaceutical compositions that contain such molecules in the treatment of inflammatory diseases or conditions.

### Description of Related Art

I. The Fcγ Receptors and CD32B

The interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation and antibody secretion. All these interactions are initiated through the binding of the Fc Domain of antibodies or immune complexes to specialized cell-surface receptors on hematopoietic cells. The diversity of cellular responses triggered by antibodies and immune complexes results from the structural heterogeneity of Fc receptors. Fc receptors share structurally related ligand binding domains which presumably mediate intracellular signaling.

The Fc receptors are members of the immunoglobulin gene superfamily of proteins. They are surface glycoproteins that can bind the Fc portion of immunoglobulin molecules. Each member of the family recognizes immunoglobulins of one or more isotypes through a recognition domain on the a chain of the Fc receptor.

Fc receptors are defined by their specificity for immunoglobulin subtypes (see, Ravetch J. V. et al. (1991) “Fc Receptors,” Annu. Rev. Immunol. 9:457-92; Gerber J. S. et al. (2001) “Stimulatory And Inhibitory Signals Originating From The Macrophage Fcγ Receptors,” Microbes and Infection, 3:131-139; Billadeau D. D. et al. (2002) “ITAMs Versus ITIMs: Striking A Balance During Cell Regulation,” J. Clin. Invest. 2(109):161-1681; Ravetch J. V. et al. (2000) “Immune Inhibitory Receptors,” Science 290:84-89; Ravetch J. V. et al. (2001) “IgG Fc Receptors,” Annu. Rev. Immunol. 19:275-90; Ravetch J. V. (1994) “Fc Receptors: Rubor Redux,” Cell, 78(4): 553-60).

Fc receptors that are capable of binding to IgG antibodies are termed “FcγRs.” Each member of this family is an integral membrane glycoprotein, possessing extracellular domains related to a C2-set of immunoglobulin-related domains, a single membrane spanning domain and an intracytoplasmic domain of variable length. There are three known FcγRs, designated FcγRI(CD64), FcγRII(CD32), and FcγRIII(CD16). The three receptors are encoded by distinct genes; however, the extensive homologies between the three family members suggest they arose from a common progenitor perhaps by gene duplication.

FcγRII(CD32) proteins are 40 KDa integral membrane glycoproteins which bind only the complexed IgG due to a low affinity for monomeric Ig (10⁶ M⁻¹). This receptor is the most widely expressed FcγR, present on all hematopoietic cells, including monocytes, macrophages, B-cells, NK cells, neutrophils, mast cells, and platelets. FcγRII has only two immunoglobulin-like regions in its immunoglobulin binding chain and hence a much lower affinity for IgG than FcγRI. There are three human FcγRII genes (FcγRIIA(CD32A), FcγRIIB(CD32B), FcγRIIC(CD32C)), all of which bind IgG in aggregates or immune complexes.

Distinct differences within the cytoplasmic domains of the FcγRIIA and FcγRIIB create two functionally heterogeneous responses to receptor ligation. The fundamental difference is that, upon binding to an IgG Fc Region, the FcγRIIA isoform initiates intracellular signaling leading to immune system activation (e.g., phagocytosis, respiratory burst, etc.), whereas, upon binding to an IgG Fc Region, the FcγRIIB isoform initiates signals that lead to the dampening or inhibition of the immune system (e.g., inhibiting B-cell activation, etc.).

Such activating and inhibitory signals are both transduced through the FcγRs following ligation to an IgG Fc Region. These diametrically opposing functions result from structural differences among the different receptor isoforms. Two distinct domains within the cytoplasmic signaling domains of the receptor called Immunoreceptor Tyrosine-based Activation Motifs (ITAMs) or Immunoreceptor Tyrosine-Based Inhibitory Motifs (ITIMS) account for the different responses. The recruitment of different cytoplasmic enzymes to these structures dictates the outcome of the FcγR-mediated cellular responses. ITAM-containing FcγR complexes include FcγRI, FcγRIIA, FcγRIIIA, whereas ITIM-containing complexes only include FcγRIIB

Human neutrophils express the FcγRIIA gene. FcγRIIA clustering via immune complexes or specific antibody cross-linking serves to aggregate ITAMs along with receptor-associated kinases which facilitate ITAM phosphorylation. ITAM phosphorylation serves as a docking site for Syk kinase, activation of which results in activation of downstream substrates (e.g., PI₃K). Cellular activation leads to release of pro-inflammatory mediators.

The FcγRIIB gene is expressed on B lymphocytes; its extracellular domain is 96% identical to FcγRIIA and binds IgG complexes in an indistinguishable manner. The presence of an ITIM in the cytoplasmic domain of FcγRIIB defines this inhibitory subclass of FcγR. The molecular basis of this inhibition has been established. When FcγRIIB becomes co-ligated to an activating receptor by way of the Fc regions of the IgG immunoglobulins of an immune complex, the FcγRIIB ITIM becomes phosphorylated and attracts the SH2 domain of the inositol polyphosphate 5′-phosphatase (SHIP), which hydrolyzes phosphoinositol messengers released as a consequence of ITAM-containing, FcγR-mediated tyrosine kinase activation, consequently preventing the influx of intracellular Ca′. Thus such cross-linking of FcγRIIB and an activating receptor dampens the activity of the activating receptor, and thus inhibits cellular responsiveness. Thus, on B-cells, B-cell activation, B-cell proliferation and antibody secretion is dampened or aborted. Thus, at the onset of antigen detection, monomeric IgG-antigen bonding occurs, and the Fc regions of bound antibodies bind to ITAMs of the activating FcγRs to mediate activation of the immune system. As the host's response progresses, multimeric IgG-antigen immune complexes form that are capable of binding to FcγRIIB (thus co-ligating such complexes with an activating receptor), leading to the dampening and ultimate cessation of the immune response (see, e.g., U.S. Pat. Nos. 8,445,645; 8,217,147; 8,216,579; 8,216,574; 8,193,318; 192,737; 8,187,593; 8,133,982; 8,044,180; 8,003,774; 7,960,512; 7,786,270; 7,632,497; 7,521,542; 7,425,619; 7,355,008 and United States Patent Publications No.: 2012/0276094; 2012/0269811; 2012/0263711; 2012/0219551; 2012/0213781; 2012/0141476; 2011/0305714; 2011/0243941; 2010/0322924; 2010/0254985; 2010/0196362; 2010/0174053; 2009/0202537; 2009/0191195; 2009/0092610; 2009/0076251; 2009/0074771; 2009/0060910; 2009/0053218; 2009/0017027; 2009/0017026; 2009/0017023; 2008/0138349; 2008/0138344; 2008/0131435; 2008/0112961; 2008/0044429; 2008/0044417; 2007/0077246; 2007/0036799; 2007/0014795; 2007/0004909; 2005/0260213; 2005/0215767; 2005/0064514; 2005/0037000; 2004/0185045).

II. The B-Cell Receptor and CD79B

B-cells are immune system cells that are responsible for producing antibodies. Additionally, B-cells present antigens and secrete cytokines. The B-cell response to antigen is an essential component of the normal immune system. B-cells possess specialized cell-surface receptors (B-cell receptors; “BCR”). If a B-cell encounters an antigen capable of binding to that cell's BCR, the B-cell will be stimulated to proliferate and produce antibodies specific for the bound antigen. To generate an efficient response to antigens, BCR-associated proteins and T-cell assistance are also required. The antigen/BCR complex is internalized, and the antigen is proteolytically processed. A small part of the antigen remains complexed with major histocompatability complex-II (“MI-IC-II”) molecules on the surface of the B-cells where the complex can be recognized by T-cells. T-cells activated by such antigen presentation secrete CD40L and a variety of lymphokines that induce B-cell maturation.

Signaling through the BCR plays an important role in the generation of antibodies, in autoimmunity, and in the establishment of immunological tolerance (Gauld, S. B. et al. (2002) “B Cell Antigen Receptor Signaling: Roles In Cell Development And Disease,” Science 296(5573):1641-1642). Immature B-cells that bind self-antigens while still in the bone marrow are eliminated by apoptosis. In contrast, antigen binding on mature B-cells results in activation, proliferation, anergy and apoptosis. The particular functional response observed depends upon whether the B-cell receives co-stimulatory signals through other surface receptors and the specific signal transduction pathways that are activated.

The BCR is composed of a membrane immunoglobulin which, together with non-covalently associated α and β subunits of CD79 (“CD79a” and “CD79B,” respectively), forms the BCR complex. CD79a and CD79B are signal transducing subunits that contain a conserved immunoreceptor tyrosine-based activation motif (“ITAM”) required for signal transduction (Dylke, J. et al. (2007) “Role of the extracellular and transmembrane domain of Ig-alpha/beta in assembly of the B cell antigen receptor (BCR),” Immunol. Lett. 112(1):47-57; Cambier, J. C. (1995) “New Nomenclature For The Reth Motif (or ARH1/TAM/ARAM/YXXL),” Immunol. Today 16:110). Aggregation of the BCR complex by multivalent antigen initiates transphosphorylation of the CD79a and CD79B ITAMs and activation of receptor-associated kinases (DeFranco, A. L. (1997) “The Complexity Of Signaling Pathways Activated By The BCR,” Curr. Opin. Immunol. 9:296-308; Kurosaki, T. (1997) “Molecular Mechanisms In B-Cell Antigen Receptor Signaling,” Curr. Opin. Immunol. 9:309-318; Kim, K. M. et al. (1993) “Signalling Function Of The B-Cell Antigen Receptors,” Immun. Rev. 132:125-146). Phosphorylated ITAMs recruit additional effectors such as PI₃K, PLC-γ and members of the Ras/MAPK pathway. These signaling events are responsible for both the B-cell proliferation and increased expression of activation markers (such as MHC-II and CD86) that are required to prime B-cells for their subsequent interactions with T-helper (“Tₕ”) cells.

III. Inflammatory Diseases or Conditions

Inflammation is a process by which the body's white blood cells and chemicals protect our bodies from infection by foreign substances, such as bacteria and viruses. It is usually characterized by pain, swelling, warmth and redness of the affected area. Chemicals known as cytokines and prostaglandins control this process, and are released in an ordered and self-limiting cascade into the blood or affected tissues. This release of chemicals increases the blood flow to the area of injury or infection, and may result in the redness and warmth. Some of the chemicals cause a leak of fluid into the tissues, resulting in swelling. This protective process may stimulate nerves and cause pain. These changes, when occurring for a limited period in the relevant area, work to the benefit of the body.

Inflammatory diseases or conditions reflect an immune system attack on a body's own cells and tissue (i.e., an “autoimmune” response). There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin. Examples of autoimmune disorders include, but are not limited to, Addison's disease, autoimmune hepatitis, autoimmune inner ear disease myasthenia gravis, Crohn's disease, dermatomyositis, familial adenomatous polyposis, graft vs. host disease (GvHD), Graves' disease, Hashimoto's thyroiditis, lupus erythematosus, multiple sclerosis (MS); pernicious anemia, Reiter's syndrome, rheumatoid arthritis (RA), Sjogren's syndrome, systemic lupus erythematosus (SLE), type 1 diabetes, primary vasculitis (e.g., polymyalgia rheumatic, giant cell arteritis, Behcets), pemphigus, neuromyelitis optica, anti-NMDA receptor encephalitis, Guillain-Barré syndrome, chronic inflammatory demyelinating polyneuropathy (CIDP), Grave's opthalmopthy, IgG4 related diseases, idiopathic thrombocytopenic purpura (ITP), and ulcerative colitis

Inflammatory diseases or conditions can also arise when the body's normally protective immune system causes damage by attacking foreign cells or tissues whose presence is beneficial to the body (e.g., the rejection of transplants (host vs. host disease)) or from the rejection of the cells of an immunosuppressed host by immunocompetent cells of an introduced transplant graft (graft vs. host disease) (DePaoli, A. M. et al. (1992) “Graft-Versus-Host Disease And Liver Transplantation,” Ann. Intern. Med. 117:170-171; Sudhindran, S. et al. (2003) “Treatment Of Graft-Versus-Host Disease After Liver Transplantation With Basiliximab Followed By Bowel Resection,” Am J Transplant. 3:1024-1029; Pollack, M. S. et al. (2005) “Severe, Late-Onset Graft-Versus-Host Disease In A Liver Transplant Recipient Documented By Chimerism Analysis,” Hum. Immunol. 66:28-31; Perri, R. et al. (2007) “Graft Vs. Host Disease After Liver Transplantation: A New Approach Is Needed,” Liver Transpl. 13:1092-1099; Mawad, R. et al. (2009) “Graft-Versus-Host Disease Presenting With Pancytopenia After En Bloc Multiorgan Transplantation: Case Report And Literature Review,” Transplant Proc. 41:4431-4433; Akbulut, S. et al. (2012) “Graft-Versus-Host Disease After Liver Transplantation: A Comprehensive Literature Review,” World J. Gastroenterol. 18(37): 5240-5248).

Despite recent advances in the treatment of such diseases or conditions, a need continues to exist for compositions capable of treating or preventing inflammatory diseases or conditions.

IV. Bispecific Binding Molecules

A. Bispecific Antibodies

The ability of an unmodified natural antibody (e.g., an IgG) to bind an epitope of an antigen depends upon the presence and interaction of Variable Domains on the immunoglobulin Light and Heavy Chains (i.e., its Light Chain Variable Domain (VL Domain) and its Heavy Chain Variable Domain (VII Domain) to form the epitope-binding sites of the antibody. As a consequence of the presence of only a single species of Light Chain and a single species of Heavy Chain, natural antibodies are capable of binding to only one epitope species (i.e., they are monospecific), although they can bind multiple copies of that species (i.e., exhibiting bi-valency or multivalency).

The art has, however, succeeded in producing bispecific antibodies, for example through the co-expression of two immunoglobulin Heavy Chain-Light Chain pairs having different epitope specificities, followed by purification of the desired molecule using affinity chromatography, as described by Milstein et al. (1983) “Hybrid Hybridomas And Their Use In Immunohistochemistry,” Nature 305:537-39, WO 93/08829, In a different approach, antibody Variable Domains with the desired binding specificities (antibody-antigen combining sites) have been fused to immunoglobulin Constant Domain sequences, for example to a Heavy Chain Constant Domain, comprising at least part of the hinge, C_{H2}, and C_{H3} regions. The nucleic acids encoding these fusions may be inserted into the same or different expression vectors, and are expressed in a suitable host organism. Bispecific antibodies are reviewed by, for example: Traunecker et al. (1991) “Bispecific Single Chain Molecules (Janusins) Target Cytotoxic Lymphocytes On HIV Infected Cells,” EMBO J. 10:3655-3659; Zhukovsky, E. A. et al. (2016) “Bispecific Antibodies And Cars: Generalized Immunotherapeutics Harnessing T Cell Redirection,” Curr. Opin. Immunol. 40:24-35; Kiefer, J. D. et al. (2016) “Immunocytokines And Bispecific Antibodies: Two Complementary Strategies For The Selective Activation Of Immune Cells At The Tumor Site,” Immunol. Rev. 270(1):178-192; Solimando, A. G. et al. (2016) “Targeting B-Cell Non-Hodgkin Lymphoma: New And Old Tricks,” Leuk. Res. 42:93-104; Fan, G. et al. (2015) “Bispecific Antibodies And Their Applications,” J. Hematol. Oncol. 8:130; Grandjenette, C. et al. (2015) “Bispecific Antibodies: An Innovative Arsenal To Hunt, Grab And Destroy Cancer Cells,” Curr. Pharm. Biotechnol. 16(8):670-683; Nuñez-Prado, N. et al. (2015) “The Coming Of Age Of Engineered Multivalent Antibodies,” Drug Discov. Today 20(5):588-594; and Kontermann, R. E. et al. (2015) “Bispecific Antibodies,” Drug. Discov. Today 20(7):838-847.

In addition to intact bispecific antibodies, the art has developed bispecific single-chain antibody derivatives (e.g., Bispecific T-cell Engagers (BiTEs)) that are composed of a single polypeptide chain having a VL and VH Domain for a first binding molecule and a VL and VH Domain for a second binding molecule (e.g., U.S. Pat. Nos. 7,112,324, 7,235,641, 7,575,923, 7,919,089; Wu, J. et al. (2015) “Blinatumomab: A Bispecific T Cell Engager (BiTe) Antibody Against CD19/CD3 For Refractory Acute Lymphoid Leukemia,” J. Hematol. Oncol. 8:104; Lutterbuese, R. et al. (2008) “Conversion Of Cetuximab, Panitumumab, Trastuzumab And Omalizumab Into T-Cell-Engaging BiTE Antibodies Creates Novel Drug Candidates Of High Potency,” Proc. Am. Assoc. Cancer Res 99:Abs 2402; Baeuerle, P. A. et al. (2009) “Bispecific T-Cell Engaging Antibodies For Cancer Therapy,” Cancer Res. 69(12):4941-4944;

B. Bispecific Diabodies

The art has noted the capability to produce diabodies that differ from natural antibodies in being capable of binding two or more different epitope species (i.e., exhibiting bispecificity or multispecificity in addition to bi-valency or multivalency). The design of a diabody is based on the single chain Fv construct (scFv), which possess a VL Domain and a corresponding VH Domain, separated by an intervening linker that allows such domains to interact with one another. Where such interaction of the VL and VH Domains is rendered impossible due to the use of a linker of insufficient length (less than about 12 amino acid residues), two such scFv constructs can interact with one another to form a bivalent diabody molecule in which the VL Domain of one chain associates with the VH Domain of the other (reviewed in Marvin et al. (2005) “Recombinant Approaches To IgG-Like Bispecific Antibodies,” Acta Pharmacol. Sin. 26:649-658, Holliger et al. (1993) “‘Diabodies’: Small Bivalent And Bispecific Antibody Fragments,” Proc. Natl. Acad. Sci. (U.S.A.) 90:6444-6448; US 2004/0058400 (Holliger et al.); US 2004/0220388 (Mertens et al.); Mertens, N. et al., “New Recombinant Bi- and Trispecific Antibody Derivatives,” In: NOVEL FRONTIERS IN THE PRODUCTION OF COMPOUNDS FOR BIOMEDICAL USE, A. VanBroekhoven et al. (Eds.), Kluwer Academic Publishers, Dordrecht, The Netherlands (2001), pages 195-208; Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng. Des. Sel. 17(1):21-27; Wu, A. et al. (2001) “Multimerization Of A Chimeric Anti-CD20 Single Chain Fv-Fc Fusion Protein Is Mediated Through Variable Domain Exchange,” Protein Engineering 14(2):1025-1033; Asano et al. (2004) “A Diabody For Cancer Immunotherapy And Its Functional Enhancement By Fusion Of Human Fc Region,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Baeuerle, P. A. et al. (2009) “Bispecific T-Cell Engaging Antibodies For Cancer Therapy,” Cancer Res. 69(12):4941-4944).

The provision of non-monospecific diabodies provides a significant advantage: the capacity to co-ligate and co-localize cells that express different epitopes. Bivalent diabodies thus have wide-ranging applications including therapy and immunodiagnosis. Bi-valency allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221). Of particular importance is the co-ligating of differing cells, for example, the cross-linking of cytotoxic T-cells to tumor cells (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305).

Diabody epitope-binding domains may also be directed to a surface determinant of any immune effector cell such as CD3, CD16, CD32, or CD64, which are expressed on T lymphocytes, natural killer (NK) cells or other mononuclear cells. In many studies, diabody binding to effector cell determinants, e.g., Fcγ receptors (FcγR), was also found to activate the effector cell (Holliger et al. (1996) “Specific Killing Of Lymphoma Cells By Cytotoxic T-Cells Mediated By A Bispecific Diabody,” Protein Eng. 9:299-305; Holliger et al. (1999) “Carcinoembryonic Antigen (CEA)-Specific T-cell Activation In Colon Carcinoma Induced By Anti-CD3 x Anti-CEA Bispecific Diabodies And B7 x Anti-CEA Bispecific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen bound antibody to an effector cell via Fc-FcγR interaction; thus, in this regard, diabody molecules of the invention may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of the tumor cells but leads to effective tumor killing (see e.g., Cao et al. (2003) “Bispecific Antibody Conjugates In Therapeutics,” Adv. Drug. Deliv. Rev. 55:171-197).

However, the above advantages come at salient cost. The formation of such non-monospecific diabodies requires the successful assembly of two or more distinct and different polypeptides (i.e., such formation requires that the diabodies be formed through the heterodimerization of different polypeptide chain species). This fact is in contrast to monospecific diabodies, which are formed through the homodimerization of identical polypeptide chains. Because at least two dissimilar polypeptides (i.e., two polypeptide species) must be provided in order to form a non-monospecific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Region,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, the art has recognized that bispecific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-monospecific diabodies (see, e.g., WO 2006/113665; WO 2008/157379; WO 2010/080538; WO 2012/018687; WO 2012/162068; Johnson, S. et al. (2010) “Effector Cell Recruitment With Novel Fv-Based Dual-Affinity Re-Targeting Protein Leads To Potent Tumor Cytolysis And In Vivo B-Cell Depletion,” J. Molec. Biol. 399(3):436-449; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7): 1933-1943; Moore, P. A. et al. (2011) “Application Of Dual Affinity Retargeting Molecules To Achieve Optimal Redirected T-Cell Killing Of B-Cell Lymphoma,” Blood 117(17):4542-4551; Chen, X. et al. (2016) “Mechanistic Projection Of First In Human Dose For Bispecific Immuno-Modulatory P-Cadherin LP-DART—An Integrated PK/PD Modeling Approach,” Clin. Pharmacol. Ther. doi: 10.1002/cpt.393; Tsai, P. et al. (2016) “CD19x CD3 DART Protein Mediates Human B-Cell Depletion In Vivo In Humanized BLT Mice,” Mol. Ther. Oncolytics. 3:15024; Root et al. (2016) “Development of PF-06671008, a Highly Potent Anti-P-cadherin/Anti-CD3 Bispecific DART Molecule with Extended Half-Life for the Treatment of Cancer,” Antibodies 5:6; Sloan, D. D. et al. (2015) “Targeting HIV Reservoir in Infected CD4 T Cells by Dual-Affinity Re-targeting Molecules (DARTs) that Bind HIV Envelope and Recruit Cytotoxic T Cells,” PLoS Pathog. 11(11):e1005233; Al-Hussaini, M. et al. (2016) “Targeting CD123 In Acute Myeloid Leukemia Using A T-Cell-Directed Dual-Affinity Retargeting Platform,” Blood 127(1):122-131); Chichili, G. R. et al. (2015) “A CD3x CD123 Bispecific DART For Redirecting Host T Cells To Myelogenous Leukemia: Preclinical Activity And Safety In Nonhuman Primates,” Sci. Transl. Med. 7(289):289ra82; Zanin, M. et al. (2015) “An Anti-H5N1 Influenza Virus FcDART Antibody Is A Highly Efficacious Therapeutic Agent And Prophylactic Against H5N1 Influenza Virus Infection,” J. Virol. 89(8):4549-4561). Such approaches involve engineering one or more cysteine residues into each of the employed polypeptide species. For example, the addition of a cysteine residue to the C-terminus of such constructs has been shown to allow disulfide bonding between the polypeptide chains, stabilizing the resulting heterodimer without interfering with the binding characteristics of the bivalent molecule.

Building on such success, the art has produced MGD010, a bispecific, bivalent DART® diabody that co-ligates the inhibitory Fcγ receptor IIb (CD32B) and the B-cell receptor (BCR) component, CD79B, on B-cells, so as to be capable of simultaneously binding CD32B and CD79B (Chen, W. (2014) “Development Of Human B-Lymphocyte Targeted Bi-Specific DART® Molecules For The Treatment Of Autoimmune Disorders,” J. Immunol. 192(1 Supp.):200.9) (FIG. 1A). The present invention relates to improved methods for using and administering MGD010 and other CD32B x CD79B bispecific molecules, particularly such bispecific molecules comprising an Fc Domain.

### SUMMARY OF THE INVENTION

The present invention is directed to methods for using bispecific binding molecules that possess a binding site specific for an epitope of CD32B and a binding site specific for an epitope of CD79B, and are thus capable of simultaneous binding to CD32B and CD79B. The invention particularly concerns such molecules that are bispecific antibodies (i.e., “CD32B x CD79B antibodies”) or bispecific diabodies (i.e., “CD32B x CD79B diabodies,” and especially such diabodies that additionally comprise an Fc Domain (i.e., “CD32B x CD79B Fc diabodies”). The invention is directed to the use of such molecules, and to the use of pharmaceutical compositions that contain such molecules in the treatment of inflammatory diseases or conditions.

In detail, the invention provides a method of treating an inflammatory disease or condition that comprises administering a therapeutically effective amount of a CD32B x CD79B Binding Molecule to a subject in need thereof, wherein the CD32B x CD79B Binding Molecule is capable of immunospecifically binding an epitope of CD32B and an epitope of CD79B, and wherein the CD32B x CD79B Binding Molecule is administered at a dose of between about 3 mg/kg and about 30 mg/kg, and at a dosage regimen of between one dose per week and one dose per 8 weeks.

The invention further concerns a method of reducing or inhibiting an immune response that comprises administering a therapeutically effective amount of a CD32B x CD79B Binding Molecule to a subject in need thereof, wherein the CD32B x CD79B Binding Molecule is capable of immunospecifically binding an epitope of CD32B and an epitope of CD79B, and wherein the CD32B x CD79B Binding Molecule is administered at a dose of between about 3 mg/kg and about 30 mg/kg, and at a dosage regimen of between one dose per week and one dose per 8 weeks.

The invention further concerns the embodiments of such methods wherein the CD32B x CD79B Binding Molecule is administered at a dose of about 3 mg/kg, wherein the CD32B x CD79B Binding Molecule is administered at a dose of about 10 mg/kg, or wherein the CD32B x CD79B Binding Molecule is administered at a dose of about 30 mg/kg.

The invention further concerns the embodiments of such methods wherein the dosage regimen is one dose per 2 weeks (Q2W), wherein the dosage regimen is one dose per 3 weeks (Q3W), or wherein the dosage regimen is one dose per 4 weeks (Q4W).

The invention further concerns the embodiments of such methods wherein the CD32B x CD79B Binding Molecule is a bispecific antibody that binds an epitope of CD32B and an epitope of CD79B, or a molecule that comprises the CD32B- and CD79B-binding domains of the bispecific antibody.

The invention further concerns the embodiments of such methods wherein the CD32B x CD79B Binding Molecule is a CD32B x CD79B bispecific diabody that binds an epitope of CD32B and an epitope of CD79B, and in particular, wherein the CD32B x CD79B bispecific diabody is a CD32B x CD79B bispecific Fc diabody.

The invention further concerns the embodiments of such methods wherein the inflammatory disease or condition is an autoimmune disease, and in particular, wherein the autoimmune disease is selected from the group consisting of: Addison's disease, autoimmune hepatitis, autoimmune inner ear disease myasthenia gravis, Crohn's disease, dermatomyositis, familial adenomatous polyposis, graft vs. host disease (GvHD), Graves' disease, Hashimoto's thyroiditis, lupus erythematosus, multiple sclerosis (MS); pernicious anemia, Reiter's syndrome, rheumatoid arthritis (RA), Sjogren's syndrome, systemic lupus erythematosus (SLE), type 1 diabetes, primary vasculitis (e.g., polymyalgia rheumatic, giant cell arteritis, Behcets), pemphigus, neuromyelitis optica, anti-NMDA receptor encephalitis, Guillain-Barré syndrome, chronic inflammatory demyelinating polyneuropathy (CIDP), Grave's opthalmopthy, IgG4 related disease, idiopathic thrombocytopenic purpura (ITP), and ulcerative colitis. The invention particularly concerns the embodiments of such methods wherein the inflammatory disease or condition is GvHD, MS, RA or SLE.

The invention further concerns the embodiments of such methods wherein the serum level of an immunoglobulin is reduced by day 36 after administration of a first dose of the CD32B x CD79B Binding Molecule. The invention particularly concerns the embodiments of such methods wherein immunoglobulin is IgM, IgA or IgG.

The invention further concerns the embodiments of such methods wherein BCR-mediated peripheral B-cell activation is inhibited by 24 hours after administration of a single dose of the CD32B x CD79B Binding Molecule, wherein the B-cell activation is determined by an ex vivo calcium mobilization assay. The invention particularly concerns the embodiments of such methods wherein BCR-mediated B-cell activation is inhibited by at least 50%, and wherein the inhibition is sustained for at least 6 days.

The invention further concerns the embodiments of such methods wherein at least 20% of the CD32B x CD79B binding sites on peripheral B-cell are occupied 6 hours after administration of a first dose of the CD32B x CD79B Binding Molecule.

The invention further concerns the embodiments of such methods wherein subject is a human.

The invention particularly concerns the embodiments of all such methods wherein the CD32B x CD79B Binding Molecule that comprises:
(A) a VL_{CD32B} Domain that comprises the amino acid sequence of SEQ ID NO:30;
(B) a VH_{CD32B} Domain that comprises the amino acid sequence of SEQ ID NO:31;
(C) a VL_{CD79B} Domain that comprises the amino acid sequence of SEQ ID NO:32;
(D) a VH_{CD79B} Domain that comprises the amino acid sequence of SEQ ID NO:33.

The invention further concerns the embodiments of such methods wherein the CD32B x CD79B Fc diabody comprises:
(A) a first polypeptide chain that comprises the amino acid sequence of SEQ ID NO:39;
(B) a second polypeptide chain that comprises the amino acid sequence of SEQ ID NO:41;
(C) a third polypeptide chain that comprises the amino acid sequence of SEQ ID NO:44.

### BRIEF DESCRIPTION OF THE DRAWINGS

FIG. 1 provides a schematic of a representative covalently bonded diabody having two epitope-binding domains composed of two polypeptide chains, each having an E-coil or K-coil Heterodimer-Promoting Domain (alternative Heterodimer-Promoting Domains are provided below). A cysteine residue may be present in a linker and/or in the Heterodimer-Promoting Domain as shown in FIGS. 3A/3B. VL and VH Domains that recognize the same epitope are shown using the same shading or fill pattern.

FIG. 2 provides a schematic of a representative covalently bonded diabody molecule having two epitope-binding domains composed of two polypeptide chains, each having a CH2 and CH3 Domain, such that the associated chains form all or part of an Fc Region. VL and VH Domains that recognize the same epitope are shown using the same shading or fill pattern.

FIGS. 3A-3E provide schematics of representative covalently bonded diabody molecule having two epitope-binding domains composed of three polypeptide chains. Two orientations of the CH2-CH3 Domains are shown (FIGS. 3A/3B vs. FIGS. 3C/3D). Two of the polypeptide chains possess a CH2 and CH3 Domain, such that the associated chains form all or part of an Fc Region. The polypeptide chains comprising the VL and VH Domain each further comprise a Heterodimer-Promoting Domain and are covalently bonded to one another via a disulfide bond formed between the cysteine residues present in the linker (FIGS. 3A and 3C) or in the Heterodimer-Promoting Domain (FIGS. 3D and 3B). FIG. 3E illustrate the structure and function of an exemplary CD32B x CD79B Fc diabody having the orientation of domains shown in FIG. 3A. The diabody shown in FIG. 3E is a covalently bonded complex that comprises the three polypeptide chains of the diabody of FIG. 3A, but without the optionally present Heterodimer-Promoting Domain. The complex includes an Fc Domain that includes a CH2 and CH3 IgG Heavy Chain Domain, and binding domains specific for CD32B and for CD79B. VL and VH Domains that recognize the same epitope are shown using the same shading or fill pattern.

FIG. 4 illustrates an exemplary mechanism through which the diabodies of the present invention may mediate their inhibition of the immune system. As shown in the Figure, a diabody of the invention is capable of simultaneously binding to a CD79B molecule of a BCR and to a CD32B molecule of a B-cell, thereby co-ligating such molecules to one another. Such co-ligation serves to permit the ITIM of the CD32B molecule to become phosphorylated and to attract the SH2 domain of the inositol polyphosphate 5′-phosphatase (SHIP), which hydrolyzes phosphoinositol messengers released as a consequence of ITAM-mediated tyrosine kinase activation. Such hydrolysis inhibits the ITAM activating signal and thereby serves to attenuate B-cell activation.

FIG. 5 shows the ability of the preferred CD32B x CD79B Fc diabody to decrease xenogeneic GvHD in vivo in a murine model.

FIG. 6 shows the in vivo pharmacokinetics of an exemplary CD32B x CD79B Binding Molecule upon administration to human subjects.

FIG. 7 summarizes the ex vivo flow cytometric analysis of in vivo binding to peripheral B-cells of an exemplary CD32B x CD79B Binding Molecule upon administration to human subjects over the study course.

FIGS. 8A-8D show the peripheral B- and T-cell populations subsequent to the administration of an exemplary CD32B x CD79B Binding Molecule to human subjects over the study course, as determined by ex vivo flow cytometric analysis.

FIGS. 9A-9D show the methods and results of B-cell function studies. FIGS. 9A-9B illustrate the ex vivo experimental procedure and data analysis methods of an ex vivo calcium mobilization assay that was used to evaluate the B-cell function of recipients of an exemplary CD32B x CD79B Binding Molecule. FIGS. 9C-9D show the reduction in peak response (FIG. 9C) and the sustained reduction in overall response as measured by the Area Under the Curve (AUC; FIG. 9D) subsequent to the administration of an exemplary CD32B x CD79B Binding Molecule to human subjects over the study course.

FIGS. 10A-10C show that administration of CD32B x CD79B Binding Molecules down-regulates BCR expression on CD27⁺ memory B-cells of human subjects over the study course. FIG. 10A: membrane-bound IgG (mIgG); FIG. 10B: membrane-bound IgM; FIG. 10C: membrane-bound IgD (mIgD). Data are presented as Mean±SEM.

FIGS. 11A-11C show that administration of CD32B x CD79B Binding Molecules down-regulates BCR expression on CD27⁻ naïve B-cells of human subjects over the study course. FIG. 11A: membrane-bound IgD (mIgD); FIG. 11B: membrane-bound IgM; FIG. 11C: percent change in membrane-bound IgM (mIgM). Membrane-bound immunoglobulin levels were determined by flow cytometry. Data are presented as Mean±SEM.

FIGS. 12A-12C show that administration of CD32B x CD79B Binding Molecules modulates serum Ig levels of human subjects over the study course. FIG. 12A: Serum IgM; FIG. 12B: Serum IgA; FIG. 12C: Serum IgG. Serum immunoglobulin IgA, IgG and IgM levels were determined by ELISA. Data are presented as Mean±SEM.

FIG. 13 shows that administration of CD32B x CD79B Binding Molecules Reduces the level of the co-stimulation molecule CD40 as determined by ex vivo flow cytometric analysis of surface co-stimulation molecules of peripheral B-cells. Data are presented as Mean±SEM.

FIGS. 14A-14B show data (at two different concentration ranges) for an ex vivo saturation Eₘₐₓ PK/PD B-cell binding study of the exemplary CD32B x CD79B Fc Diabody of Example 1. Data were graphically evaluated on linear-linear and log-linear scale.

FIGS. 15A-15F depict preclinical target concentrations with superimposed CD32B x CD79B Binding Molecule pharmacokinetic profiles in humans to identify the doses that would attain the target concentrations. In FIGS. 15A-15F, the y-axes are CD32B x CD79B Binding Molecule Concentration [ng/mL] and the x-axes are time in hours, the top, middle and lower horizontal lines are, respectively, the Binding Molecule concentration values of in vitro B-cell binding/inhibition studies, the Binding Molecule concentration values of EC50 B-cell binding in the current study, and the Binding Molecule concentration values of in vivo inhibition of IgG and IgM in a humanized mouse model.

FIGS. 16A-16D show simulations of the mean concentration of CD32B x CD79B Binding Molecule at doses of 0.3 mg/kg (FIG. 16A), 1 mg/kg (FIG. 16B), 3 mg/kg (FIG. 16C) and 10 mg/kg (FIG. 16D) subject body weight for once per 2 week (Q2W), once per 3 week (Q3W) and once per 4 week (Q4W) dosing regimens. Actual times and concentrations and nominal doses were used. For FIGS. 16A-16D, the y-axes are CD32B x CD79B Binding Molecule Concentration [ng/mL] and the x-axes are time in hours.

FIGS. 17A-17D show the predicted variability (with SD) in the modeled profiles of FIGS. 16A-16D.

FIG. 18 shows the concentration of HAV-specific IgG present in the serum of healthy human subjects at day 57 after vaccination with HAV. These data show that administration of CD32B x CD79B Binding Molecules reduces HAV-specific IgG levels in HAV-vaccinated human subjects.

FIG. 19 shows that CD32B x CD79B Binding Molecules block CD40 dependent B-Cell responses as determine by in vitro detection of CD40 dependent B-cell IgG secretion. Human B-cells were cultured with or without stimulators (CD40-ligand (500 ng/mL), IL-4 (100 ng/mL) and IL-21 (20 ng/mL)) used undiluted, or a series of 3 fold dilutions (1, 1/3, 1/9, and 1/27) in the presence or absence of the exemplary CD32B x CD79B Binding Molecule (20 μg/mL) for 5 days and secreted IgG was determined by ELISA assay.

### DETAILED DESCRIPTION OF THE INVENTION

The present invention is directed to methods for using bispecific binding molecules that possess a binding site specific for an epitope of CD32B and a binding site specific for an epitope of CD79B, and are thus capable of simultaneous binding to CD32B and CD79B. The invention particularly concerns such molecules that are bispecific antibodies (i.e., “CD32B x CD79B antibodies”) or bispecific diabodies (i.e., “CD32B x CD79B diabodies,” and especially such diabodies that additionally comprise an Fc Domain (i.e., “CD32B x CD79B Fc diabodies”). The invention is directed to the use of such molecules, and to the use of pharmaceutical compositions that contain such molecules.

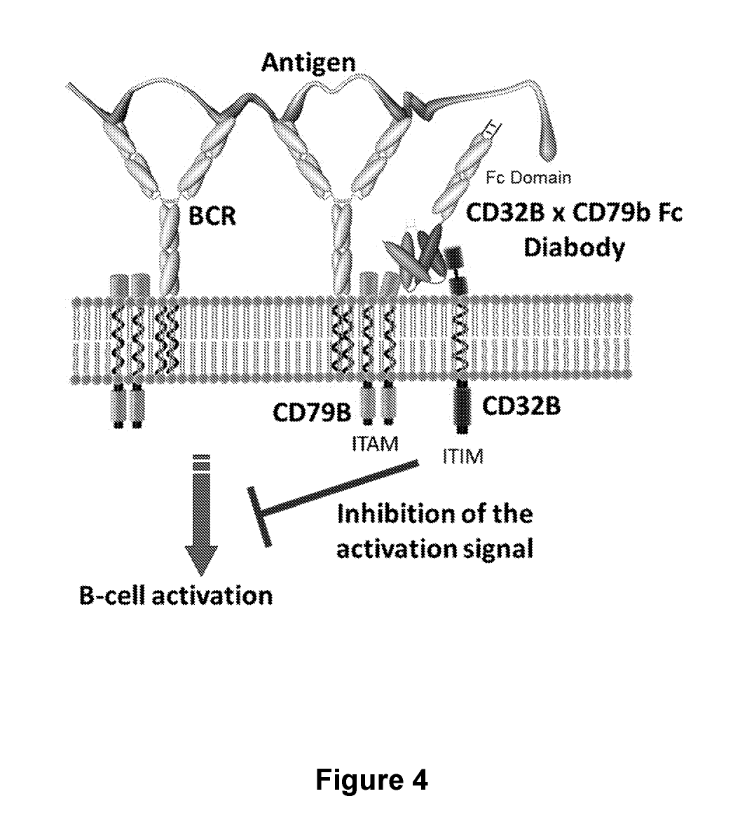

As discussed above, CD79B and CD32B (FcγRIIB) are both expressed by B-cells that are proliferating in response to antigen recognition. The bispecific binding molecules of the invention are capable of immunospecifically binding to both molecules and are thus capable of co-ligating the molecules. Such co-ligation (see, e.g., FIG. 4) permits the ITIM of the CD32B molecule to become phosphorylated and to attract the SH2 domain of the inositol polyphosphate 5′-phosphatase (SHIP), which hydrolyzes phosphoinositol messengers that are released as a consequence of the tyrosine kinase-mediated activation of the CD79B ITAM. Such hydrolysis inhibits the ITAM activating signal of CD79B and thereby serves to attenuate B-cell activation. Thus, the bispecific binding molecules of the invention have the ability to inhibit or dampen a host's immune system in response to an unwanted B-cell activation, B-cell proliferation and antibody secretion, and have utility in the treatment of inflammatory diseases and disorders and in particular, systemic lupus erythematosus (SLE), multiple sclerosis (MS), and graft vs. host disease (GvHD).

I. Antibody Characteristics and Structure

As used herein, the term “antibody” refers to an immunoglobulin molecule capable of immunospecific binding to a polypeptide or protein or a non-protein molecule due to the presence on such molecule of a particular domain or moiety or conformation (an “epitope”). An epitope-containing molecule may have immunogenic activity, such that it elicits an antibody production response in an animal; such molecules are termed “antigens”). Epitope-containing molecules need not necessarily be immunogenic.

Natural antibodies (such as IgG antibodies) are composed of two Light Chains complexed with two Heavy Chains. Each Light Chain of a natural antibody (such as an IgG antibody) contains a Variable Domain (VL Domain) and a Constant Domain (CL Domain). Each Heavy Chain of a natural antibody contains a Heavy Chain Variable Domain (VH Domain), three Constant Domains (CH1, CH2 and CH3 Domains), and a “Hinge” Domain (“H”) located between the CH1 and CH2 Domains. The basic structural unit of naturally occurring immunoglobulins (e.g., IgG) is thus a tetramer having two Light Chains and two Heavy Chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with Light Chains having a single Constant Domain and Heavy Chains usually having three Constant Domains and a Hinge Domain. Thus, the structure of the Light Chains of an IgG molecule is n-VL-CL-c and the structure of the IgG Heavy Chains is n-VH-CH1-H-CH2-CH3-c (where n and c represent, respectively, the N-terminus and the C-terminus of the polypeptide). The ability of an antibody to bind an epitope of an antigen depends upon the presence and amino acid sequence of the antibody's VL and VH Domains. Interaction of an antibody Light Chain and an antibody Heavy Chain and, in particular, interaction of its VL and VH Domains forms one of the two epitope-binding sites of a natural antibody. Natural antibodies are capable of binding to only one epitope species (i.e., they are monospecific), although they can bind multiple copies of that species (i.e., exhibiting bi-valency or multivalency). The Variable Domains of an IgG molecule consist of the complementarity determining regions (CDR), which contain the residues in contact with epitope, and non-CDR segments, referred to as framework segments (FR), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. Polypeptides that are (or may serve as) the first, second and third CDR of an antibody Light Chain are herein respectively designated CDR_{L}1 Domain, CDR_{L}2 Domain, and CDR_{L}3 Domain. Similarly, polypeptides that are (or may serve as) the first, second and third CDR of an antibody Heavy Chain are herein respectively designated CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and Heavy Chains or a diabody or a single-chain binding molecule (e.g., an scFv, a BiTe, etc.), or is another type of protein. Accordingly, as used herein, the term “Epitope-Binding Domain” refers to that portion of an epitope-binding molecule that is responsible for the ability of such molecule to immunospecifically bind an epitope. An epitope-binding fragment may contain 1, 2, 3, 4, 5 or all 6 of the CDR Domains of such antibody and, although capable of immunospecifically binding to such epitope, may exhibit an immunospecificity, affinity or selectivity towards such epitope that differs from that of such antibody. Preferably, however, an epitope-binding fragment will contain all 6 of the CDR Domains of such antibody. An epitope-binding fragment of an antibody may be a single polypeptide chain (e.g., an scFv), or may comprise two or more polypeptide chains, each having an amino terminus and a carboxy terminus (e.g., a diabody, a Fab fragment, an F(ab′)₂ fragment, etc.).

The term “antibody,” as used herein, encompasses monoclonal antibodies, multi specific antibodies, human antibodies, humanized antibodies, synthetic antibodies, chimeric antibodies, polyclonal antibodies, camelized antibodies, single-chain Fvs (scFv), single-chain antibodies, immunologically active antibody fragments (e.g., antibody fragments capable of binding to an epitope, e.g., Fab fragments, Fab′ fragments, F(ab′)2 fragments, Fv fragments, fragments containing a VL and/or VH Domain, or that contain 1, 2, or 3 of the complementarity determining regions (CDRs) of such VL Domain (i.e., CDR_{L}1, CDR_{L}2, and/or CDR_{L}3) or VH Domain (i.e., CDR_{H}1, CDR_{H}2, and/or CDR_{H}3)) that specifically bind an antigen, etc., bi-functional or multi-functional antibodies, disulfide-linked bispecific Fvs (sdFv), intrabodies, and diabodies, and epitope-binding fragments of any of the above. In particular, the term “antibody” is intended to encompass immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass (see, e.g., United States Patent Publication Nos.: 20040185045; 20050037000; 20050064514; 20050215767; 20070004909; 20070036799; 20070077246; and 20070244303). The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). Over 200 antibody-based drugs have been approved for use or are under development.

The term “chimeric antibody” refers to an antibody in which a portion of a heavy and/or Light Chain is identical to or homologous with an antibody from one species (e.g., mouse) or antibody class or subclass, while the remaining portion is identical to or homologous with an antibody of another species (e.g., human) or antibody class or subclass, so long as they exhibit the desired biological activity. Chimeric antibodies of interest herein include “primatized” antibodies comprising Variable Domain antigen binding sequences derived from a non-human primate (e.g., Old World Monkey, Ape, etc.) and human constant region sequences.

The term “monoclonal antibody” as used herein refers to an antibody of a population of substantially homogeneous antibodies, i.e., the individual antibodies comprising the population are identical except for possible antibodies possessing naturally occurring mutations that may be present in minor amounts, and the term “polyclonal antibody” as used herein refers to an antibody obtained from a population of heterogeneous antibodies. The term “monoclonal” indicates the character of the antibody as being a substantially homogeneous population of antibodies, and is not to be construed as requiring production of the antibody by any particular method (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125). In general, cells should be kept intact and preferably viable when used as immunogens. Intact cells may allow antigens to be better detected than ruptured cells by the immunized animal. Use of denaturing or harsh adjuvants, e.g., Freud's adjuvant, may rupture cells and therefore is discouraged. The immunogen may be administered multiple times at periodic intervals such as, bi-weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules of the invention as well as an affinity optimized, a chimeric antibody, a humanized antibody, and/or a caninized antibody, to improve the affinity, or other characteristics of the antibody.

The term “scFv” refers to single-chain Variable Domain fragments. scFv molecules are made by linking Light and/or Heavy Chain Variable Domain using a short linking peptide. Bird et al. (1988) (“Single-Chain Antigen-Binding Proteins,” Science 242:423-426) describes example of linking peptides which bridge approximately 3.5 nm between the carboxy terminus of one Variable Domain and the amino terminus of the other Variable Domain. Linkers of other sequences have been designed and used (Bird et al. (1988) “Single-Chain Antigen-Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The term “humanized antibody” refers to a chimeric molecule, generally prepared using recombinant techniques, having an epitope-binding site of an immunoglobulin from a non-human species and a remaining immunoglobulin structure of the molecule that is based upon the structure and/or sequence of a human immunoglobulin. The epitope-binding site may comprise either complete Variable Domains fused onto Constant Domains or only the CDRs grafted onto appropriate framework regions in the Variable Domains. Epitope-binding sites may be wild-type or modified by one or more amino acid substitutions. This eliminates the constant region as an immunogen in human individuals, but the possibility of an immune response to the foreign variable region remains (LoBuglio, A. F. et al. (1989) “Mouse/Human Chimeric Monoclonal Antibody In Man: Kinetics And Immune Response,” Proc. Natl. Acad. Sci. (U.S.A.) 86:4220-4224). Another approach focuses not only on providing human-derived constant regions, but modifying the variable regions as well so as to reshape them as closely as possible to human form. It is known that the variable regions of both heavy and Light Chains contain three CDRs which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable regions can be “reshaped” or “humanized” by grafting CDRs derived from a non-human antibody on the FRs present in the human antibody to be modified. Application of this approach to various antibodies has been reported by Sato, K. et al. (1993) “Reshaping A Human Antibody To Inhibit The Interleukin 6-Dependent Tumor Cell Growth,” Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five or six) that are altered in their amino acid sequence(s) relative to the original antibody, which are also termed one or more CDRs “derived from” one or more CDRs from the original antibody (i.e., derived from such CDRs, derived from knowledge of the amino acid sequences of such CDRs, etc.). A polynucleotide sequence that encodes the Variable Domain of an antibody may be used to generate such derivatives and to improve the affinity, or other characteristics of such antibodies. The general principle in humanizing an antibody involves retaining the basic sequence of the epitope-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy Variable Domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

As indicated above, the Bispecific Binding Molecules of the present invention possess at least two Epitope-Binding Domains. Each of such Epitope-Binding Domains are capable of binding to epitopes in an “immunospecific” manner. As used herein, an antibody, diabody or other Bispecific Binding Molecule of the present invention is said to “immunospecifically” bind (or to exhibit “specific” binding to) a region of another molecule (i.e., an epitope) if it reacts or associates more frequently, more rapidly, with greater duration and/or with greater affinity with that epitope relative to alternative epitopes. For example, an antibody that specifically binds to an epitope of CD32B (or an epitope of CD79B) is an antibody that binds such epitope with greater affinity, avidity, more readily, and/or with greater duration than it binds to other epitopes of CD32B (or to other epitopes of CD79B) or to an epitope of a molecule other than CD32B (or CD79B). It is also understood by reading this definition that, for example, an antibody (or moiety or epitope) that immunospecifically binds to a first target may or may not specifically or preferentially bind to a second target. As such, “immunospecific binding” does not necessarily require (although it can include) exclusive binding. Generally, but not necessarily, reference to binding means “specific” binding. The ability of an antibody to immunospecifically bind to an epitope may be determined by, for example, an immunoassay.

An Epitope-Binding Domain of the humanized molecules of the present invention may comprise a complete Variable Domain fused to a Constant Domain or only the complementarity determining regions (CDRs) of such Variable Domain grafted to appropriate framework regions. An Epitope-Binding Domain may be wild-type or may be modified by one or more amino acid substitutions, for example to lessen the ability of any Constant Domain of the molecule to serve as an immunogen in human individuals. Although this may eliminate the constant region as an immunogen in human individuals, the possibility of an immune response to the foreign Variable Domain remains (LoBuglio, A. F. et al. (1989) “Mouse/Human Chimeric Monoclonal Antibody In Man: Kinetics And Immune Response,” Proc. Natl. Acad. Sci. (U.S.A.) 86:4220-4224). Another approach focuses not only on providing human-derived constant regions, but modifying the Variable Domains as well so as to reshape them as closely as possible to human form. It is known that the Variable Domains of both heavy and Light Chains contain three complementarity determining regions (CDRs) which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the Variable Domains can be “reshaped” or “humanized” by grafting CDRs derived from non-human antibody on the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which differ in sequence relative to the original antibody.

A number of “humanized” antibody molecules comprising an epitope-binding site derived from a non-human immunoglobulin have been described, including chimeric antibodies having rodent or modified rodent Variable Domain and their associated complementarity determining regions (CDRs) fused to human Constant Domains (see, for example, Winter et al. (1991) “Man-made Antibodies,” Nature 349:293-299; Lobuglio et al. (1989) “Mouse/Human Chimeric Monoclonal Antibody In Man: Kinetics And Immune Response,” Proc. Natl. Acad. Sci. (U.S.A.) 86:4220-4224 (1989), Shaw et al. (1987) “Characterization Of A Mouse/Human Chimeric Monoclonal Antibody (17-1A) To A Colon Cancer Tumor-Associated Antigen,” J. Immunol. 138:4534-4538, and Brown et al. (1987) “Tumor-Specific Genetically Engineered Murine/Human Chimeric Monoclonal Antibody,” Cancer Res. 47:3577-3583). Other references describe rodent CDRs grafted into a human supporting framework region (FR) prior to fusion with an appropriate human antibody Constant Domain (see, for example, Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; and Jones et al. (1986) “Replacing The Complementarity Determining Regions In A Human Antibody With Those From A Mouse,” Nature 321:522-525). Another reference describes rodent CDRs supported by recombinantly veneered rodent framework regions. See, for example, European Patent Publication No. 519,596. These “humanized” molecules are designed to minimize unwanted immunological response towards rodent anti-human antibody molecules, which limits the duration and effectiveness of therapeutic applications of those moieties in human recipients. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; 5,997,867; and 5,866,692.

### II. Antibody Constant Regions

A preferred CL Domain is a human IgG CL Kappa Domain. The amino acid sequence of an exemplary human CL Kappa Domain is (SEQ ID NO:1):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an exemplary CL Domain is a human IgG CL Lambda Domain. The amino acid sequence of an exemplary human CL Lambda Domain is (SEQ ID NO:2):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

An exemplary CH1 Domain is a human IgG1 CH1 Domain. The amino acid sequence of an exemplary human IgG1 CH1 Domain is (SEQ ID NO:3):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

An exemplary CH1 Domain is a human IgG2 CH1 Domain. The amino acid sequence of an exemplary human IgG2 CH1 Domain is (SEQ ID NO:4):

ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTV

An exemplary CH1 Domain is a human IgG4 CH1 Domain. The amino acid sequence of an exemplary human IgG4 CH1 Domain is (SEQ ID NO:5):

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRV

One exemplary hinge region is a human IgG1 Hinge Region. The amino acid sequence of an exemplary human IgG1 Hinge Region is (SEQ ID NO:6):

EPKSCDKTHTCPPCP.

Another exemplary hinge region is a human IgG2 Hinge Region. The amino acid sequence of an exemplary human IgG2 Hinge Region is (SEQ ID NO:7):

ERKCCVECPPCP.

Another exemplary hinge region is a human IgG4 Hinge Region. The amino acid sequence of an exemplary human IgG4 Hinge Region is (SEQ ID NO:8): ESKYGPPCPSCP. As described herein, an IgG4 hinge region may comprise a stabilizing mutation such as the S228P substitution. The amino acid sequence of an exemplary stabilized IgG4 Hinge Region is (SEQ ID NO:9): ESKYGPPCPPCP.

The CH2 and CH3 Domains of antibody Heavy Chains interact to form the Fc Region, which contains an Fc Domain that is recognized by cellular Fc Receptors, including but not limited to Fc gamma Receptors (FcγRs) such as CD32B. The amino acid sequence of the CH2-CH3 Domain of an exemplary human IgG1 is (SEQ ID NO:10):

```
231 240 250 260 270
APELLGGPSV FLFPPKPKDT LMISRTPEVT CVVVDVSHED
              280 290 300 310
PEVKFNWYVD GVEVHNAKTK PREEQYNSTY RVVSVLTVLH
              320 330 340 350
QDWLNGKEYK CKVSNKALPA PIEKTISKAK GQPREPQVYT
              360 370 380 390
LPPSREEMTK NQVSLTCLVK GFYPSDIAVE WESNGQPENN
              400 410 420 430
YKTTPPVLDS DGSFFLYSKL TVDKSRWQQG NVFSCSVMHE
              440 447
ALHNHYTQKS LSLSPGX
```

as numbered by the EU index as set forth in Kabat, wherein, X is a lysine (K) or is absent.

The amino acid sequence of the CH2-CH3 Domain of an exemplary human IgG2 is (SEQ ID NO:11):

```
231 240 250 260 270
APPVA-GPSV FLFPPKPKDT LMISRTPEVT CVVVDVSHED
              280 290 300 310
PEVQFNWYVD GVEVHNAKTK PREEQFNSTF RVVSVLTVVH
              320 330 340 350
QDWLNGKEYK CKVSNKGLPA PIEKTISKTK GQPREPQVYT
              360 370 380 390
LPPSREEMTK NQVSLTCLVK GFYPSDISVE WESNGQPENN
              400 410 420 430
YKTTPPMLDS DGSFFLYSKL TVDKSRWQQG NVFSCSVMHE
              440 447
ALHNHYTQKS LSLSPGX
```

as numbered by the EU index as set forth in Kabat, wherein, X is a lysine (K) or is absent.

The amino acid sequence of the CH2-CH3 Domain of an exemplary human IgG3 is (SEQ ID NO:12):

```
231 240 250 260 270
APELLGGPSV FLFPPKPKDT LMISRTPEVT CVVVDVSHED
              280 290 300 310
PEVQFKWYVD GVEVHNAKTK PREEQYNSTF RVVSVLTVLH
              320 330 340 350
QDWLNGKEYK CKVSNKALPA PIEKTISKTK GQPREPQVYT
              360 370 380 390
LPPSREEMTK NQVSLTCLVK GFYPSDIAVE WESSGQPENN
              400 410 420 430
YNTTPPMLDS DGSFFLYSKL TVDKSRWQQG NIFSCSVMHE
              440 447
ALHNRFTQKS LSLSPGX
```

as numbered by the EU index as set forth in Kabat, wherein, X is a lysine (K) or is absent.

The amino acid sequence of the CH2-CH3 Domain of an exemplary human IgG4 is (SEQ ID NO:13):

```
231 240 250 260 270
APEFLGGPSV FLFPPKPKDT LMISRTPEVT CVVVDVSQED
              280 290 300 310
PEVQFNWYVD GVEVHNAKTK PREEQFNSTY RVVSVLTVLH
              320 330 340 350
QDWLNGKEYK CKVSNKGLPS SIEKTISKAK GQPREPQVYT
              360 370 380 390
LPPSQEEMTK NQVSLTCLVK GFYPSDIAVE WESNGQPENN
              400 410 420 430
YKTTPPVLDS DGSFFLYSRL TVDKSRWQEG NVFSCSVMHE
              440 447
ALHNHYTQKS LSLSLGX
```

as numbered by the EU index as set forth in Kabat, wherein, X is a lysine (K) or is absent.

Throughout the present specification, the numbering of the residues in the constant region of an IgG Heavy Chain is that of the EU index as in Kabat et al., Sequences of Proteins of Immunological Interest, 5^{th} Ed. Public Health Service, NH1, MD (1991) (“Kabat”), expressly incorporated herein by references. The term “EU index as in Kabat” refers to the numbering of the human IgG1 EU antibody. Amino acids from the Variable Domains of the mature heavy and Light Chains of immunoglobulins are designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (it will be understood that CDR_{H}1 as defined by Chothia, C. & Lesk, A. M. ((1987) “Canonical structures for the hypervariable regions of immunoglobulins,”. J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody Light Chain occupies the equivalent position to an amino acid at position 50 of a mouse antibody Light Chain.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the antibodies of the present invention may be incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the Fc Domain-containing Binding Molecules of the invention. Specifically encompassed by the instant invention are Binding Molecules of the invention lacking the C-terminal residue of the CH3 Domain. Also specifically encompassed by the instant invention are such constructs comprising the C-terminal lysine residue of the CH3 Domain.

III. Preferred CD32B x CD79B Binding Molecules of the Present Invention

The present invention relates to bispecific binding molecules that are capable of binding to an epitope of CD32B and an epitope of Cd79b, so as to be capable of simultaneously binding to such molecules as natively arrayed (i.e., without recombinantly-induced overexpression) on the surface of a B-cell. Such specific binding molecules may be composed of a single polypeptide chain (e.g., a BiTe), or may be composed of two, three, four, five or more polypeptide chains that together form a covalently bonded complex, preferably through the presence of multiple disulfide bonds between individual polypeptide chains of the CD32B x CD79B Binding Molecule. Preferably, such molecules will be capable of immunospecifically binding to CD32B without substantially interfering with, or impeding, the ability of the CD32B molecule to bind to the Fc Domain of an antibody or of an Fc Domain-containing diabody.

A. Bispecific ScFv and Antibodies

In a first preferred embodiment, the CD32B x CD79B Binding Molecules of the present invention are single chain molecules, such as BiTes that possess a VL_{CD32B} Domain, a VL_{CD79B} Domain, a VH_{CD32B} Domain and a VL_{CD79B} Domain, and in which such domains are separated by peptide linker molecules that permit the VL_{CD32B} Domain to interact with the VH_{CD32B} Domain so as to form a CD32B-Epitope-Binding Domain, and that permit the VL_{CD79B} Domain to interact with the VH_{CD79B} Domain so as to form a CD79B-Epitope-Binding Domain.

In a second preferred embodiment, the CD32B x CD79B Binding Molecules of the present invention are bispecific antibodies, or epitope-binding fragments thereof, that possess a VL_{CD32B} Domain, a VL_{CD79B} Domain, a VH_{CD32B} Domain and a VL_{CD79B} Domain, so as to form a CD32B-Epitope-Binding Domain and a CD79B-Epitope-Binding Domain. Such antibodies may contain an Fc Domain.

B. Bispecific Diabodies

1. Non-Fc-Domain-Containing Bispecific Diabodies

In a further preferred embodiment, the CD32B x CD79B Binding Molecules of the present invention are bispecific monovalent diabodies that are composed of two, three, four, five or more polypeptide chains.

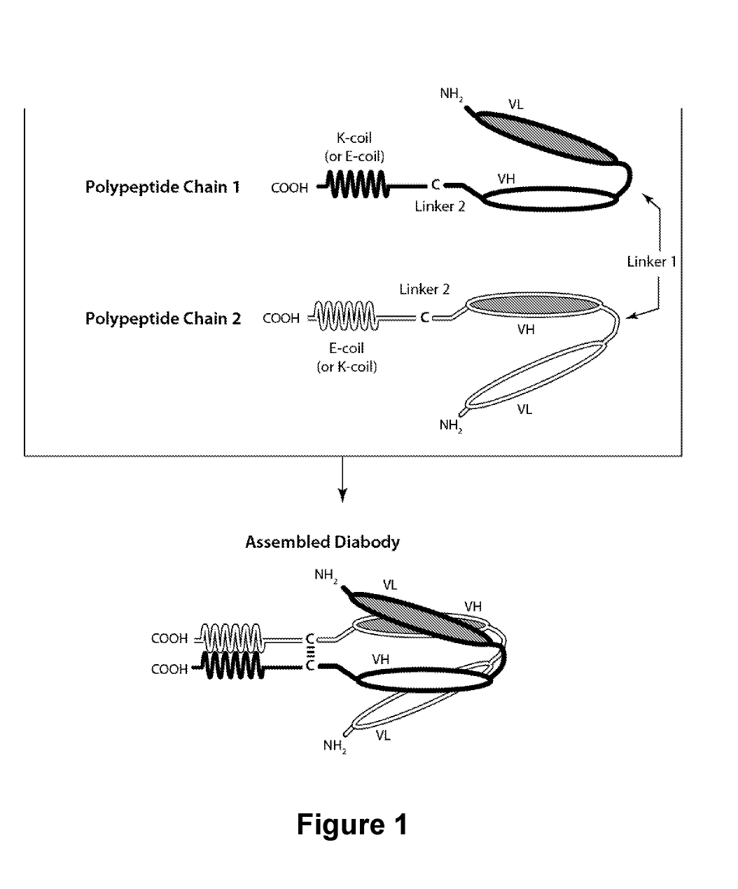

For example, FIG. 1 shows a CD32B x CD79B bispecific monovalent diabody composed of two polypeptide chains, which are covalently bonded to one another via a disulfide bond. The VL Domain of the first polypeptide chain interacts with the VH Domain of the second polypeptide chain in order to form a first functional antigen binding site that is specific for the first antigen (i.e., either CD32B or CD79B). Likewise, the VL Domain of the second polypeptide chain interacts with the VH Domain of the first polypeptide chain in order to form a second functional antigen binding site that is specific for the second antigen (i.e., either CD79B or CD32B, depending upon the identity of the first antigen). Thus, the selection of the VL and VH Domains of the first and second polypeptide chains are coordinated, such that the two polypeptide chains collectively comprise VL and VH Domains capable of binding to CD32B and CD79B (i.e., they comprise VL_{CD32B}/VH_{CD32B} and VL_{CD79B}/VH_{CD79B}) (FIG. 1). Collectively, each such VL and VH Domain, and the intervening Linker that separates them, are referred to as an Antigen-Binding Domain of the molecule.

The first polypeptide chain of the preferred CD32B x CD79B bispecific monovalent diabody comprises (in the N-terminal to C-terminal direction): an amino terminus, the VL Domain of a monoclonal antibody capable of binding to either CD32B or CD79B (i.e., either VL_{CD32B} or VL_{CD79B}), an intervening spacer peptide (Linker 1), a VH Domain of a monoclonal antibody capable of binding to either CD79B (if such first polypeptide chain contains VL_{CD32B}) or CD32B (if such first polypeptide chain contains VL_{CD79B}), an intervening spacer peptide (Linker 2), a Heterodimer-Promoting Domain, an optional further domain to provide improved stabilization to the Heterodimer-Promoting Domain and a C-terminus (FIG. 1).

The second polypeptide chain of such preferred CD32B x CD79B bispecific monovalent Fc diabody comprises (in the N-terminal to C-terminal direction): an amino terminus, a VL Domain of a monoclonal antibody capable of binding to either CD79B or CD32B (i.e., either VL_{CD79B} or VL_{CD32B}, depending upon the VL Domain selected for the first polypeptide chain of the diabody), an intervening linker peptide (Linker 1), a VH Domain of a monoclonal antibody capable of binding to either CD32B (if such second polypeptide chain contains VL_{CD79B}) or CD32B (if such second polypeptide chain contains VL_{CD32B}), an intervening spacer peptide (Linker 2), a Heterodimer-Promoting Domain, and a C-terminus (FIG. 1).

Most preferably, the length of Linker 1, which separates such VL and VH Domains is selected to substantially or completely prevent such VL and VH Domains from binding to one another (for example consisting of from 1, 2, 3, 4, 5, 6, 7, 8, or 9 amino acid residues). Thus, the VL and VH Domains of the first polypeptide chain are substantially or completely incapable of binding to one another. Likewise, the VL and VH Domains of the second polypeptide chain are substantially or completely incapable of binding to one another. A preferred intervening spacer peptide (Linker 1) has the sequence (SEQ ID NO:14): GGGSGGGG.

The purpose of Linker 2 is to separate the VH Domain of a polypeptide chain from the optionally present Heterodimer-Promoting Domain of that polypeptide chain. Any of a variety of linkers can be used for the purpose of Linker 2. A preferred sequence for such Linker 2 has the amino acid sequence: AS TKG (SEQ ID NO:15), which is derived from the IgG CH1 Domain, or GGCGGG (SEQ ID NO:16), which possesses a cysteine residue that may be used to covalently bond the first and second polypeptide chains to one another via a disulfide bond. Since the Linker 2, AS TKG (SEQ ID NO:15) does not possess such a cysteine, the use of such Linker 2 is preferably associated with the use of a cysteine-containing Heterodimer-Promoting Domain, such as the E-coil of SEQ ID NO:23 or the K-coil of SEQ ID NO:24 (see below). Thus, in one embodiment, Linker 2 of the polypeptide chains contains a cysteine residue (so as to covalently link the first and second polypeptide chains to one another). In another embodiment, Linker 2 of the polypeptide chains does not possess a cysteine, and the Heterodimer-Promoting Domains of such polypeptide chains contains such a cysteine residue to thereby covalently link the first and second polypeptide chains to one another.

The formation of heterodimers of the first and second polypeptide chains can be driven by the inclusion of Heterodimer-Promoting Domains. Such domains include GVEPKSC (SEQ ID NO:17) or VEPKSC (SEQ ID NO:18) on one polypeptide chain and GFNRGEC (SEQ ID NO:19) or FNRGEC (SEQ ID NO:20) on the other polypeptide chain (US2007/0004909).

More preferably, however, the Heterodimer-Promoting Domains of the present invention are formed from one, two, three or four tandemly repeated coil domains of opposing charge that comprise a sequence of at least six, at least seven or at least eight charged amino acid residues (Apostolovic, B. et al. (2008) “pH-Sensitivity of the E3/K3 Heterodimeric Coiled Coil,” Biomacromolecules 9:3173-3180; Arndt, K. M. et al. (2001) “Helix-stabilized Fv (hsFv) Antibody Fragments: Substituting the Constant Domains of a Fab Fragment for a Heterodimeric Coiled-coil Domain,” J. Molec. Biol. 312:221-228; Arndt, K. M. et al. (2002) “Comparison of In Vivo Selection and Rational Design of Heterodimeric Coiled Coils,” Structure 10:1235-1248; Boucher, C. et al. (2010) “Protein Detection By Western Blot Via Coiled—Coil Interactions,” Analytical Biochemistry 399:138-140; Cachia, P. J. et al. (2004) “Synthetic Peptide Vaccine Development: Measurement Of Polyclonal Antibody Affinity And Cross-Reactivity Using A New Peptide Capture And Release System For Surface Plasmon Resonance Spectroscopy,” J. Mol. Recognit. 17:540-557; De Crescenzo, G. D. et al. (2003) “Real-Time Monitoring of the Interactions of Two-Stranded de novo Designed Coiled-Coils: Effect of Chain Length on the Kinetic and Thermodynamic Constants of Binding,” Biochemistry 42:1754-1763; Fernandez-Rodriquez, J. et al. (2012) “Induced Heterodimerization And Purification Of Two Target Proteins By A Synthetic Coiled-Coil Tag,” Protein Science 21:511-519; Ghosh, T. S. et al. (2009) “End-To-End And End-To-Middle Interhelical Interactions: New Classes Of Interacting Helix Pairs In Protein Structures,” Acta Crystallographica D65:1032-1041; Grigoryan, G. et al. (2008) “Structural Specificity In Coiled-Coil Interactions,” Curr. Opin. Struc. Biol. 18:477-483; Litowski, J. R. et al. (2002) “Designing Heterodimeric Two-Stranded α-Helical Coiled-Coils: The Effects Of Hydrophobicity And α-Helical Propensity On Protein Folding, Stability, And Specificity,” J. Biol. Chem. 277:37272-37279; Steinkruger, J. D. et al. (2012) “The d′--d--d′ Vertical Triad is Less Discriminating Than the a′--a--a′ Vertical Triad in the Antiparallel Coiled-coil Dimer Motif,” J. Amer. Chem. Soc. 134(5):2626-2633; Straussman, R. et al. (2007) “Kinking the Coiled Coil—Negatively Charged Residues at the Coiled-coil Interface,” J. Molec. Biol. 366:1232-1242; Tripet, B. et al. (2002) “Kinetic Analysis of the Interactions between Troponin C and the C-terminal Troponin I Regulatory Region and Validation of a New Peptide Delivery/Capture System used for Surface Plasmon Resonance,” J. Molec. Biol. 323:345-362; Woolfson, D. N. (2005) “The Design Of Coiled-Coil Structures And Assemblies,” Adv. Prot. Chem. 70:79-112; Zeng, Y. et al. (2008) “A Ligand-Pseudoreceptor System Based On de novo Designed Peptides For The Generation Of Adenoviral Vectors With Altered Tropism,” J. Gene Med. 10:355-367).

Such repeated coil domains may be exact repeats or may have substitutions. For example, the Heterodimer-Promoting Domain of the first polypeptide chain may comprise a sequence of eight negatively charged amino acid residues and the Heterodimer-Promoting Domain of the second polypeptide chain may comprise a sequence of eight negatively charged amino acid residues. It is immaterial which coil is provided to the first or second polypeptide chains, provided that a coil of opposite charge is used for the other polypeptide chain. However, a preferred CD32B x CD79B bispecific monovalent diabody of the present invention has a first polypeptide chain having a negatively charged coil. The positively charged amino acid may be lysine, arginine, histidine, etc. and/or the negatively charged amino acid may be glutamic acid, aspartic acid, etc. The positively charged amino acid is preferably lysine and/or the negatively charged amino acid is preferably glutamic acid. It is possible for only a single Heterodimer-Promoting Domain to be employed (since such domain will inhibit homodimerization and thereby promote heterodimerization), however, it is preferred for both the first and second polypeptide chains of the diabodies of the present invention to contain Heterodimer-Promoting Domains.

In a preferred embodiment, one of the Heterodimer-Promoting Domains will comprise four tandem “E-coil” helical domains (SEQ ID NO:21: EVAALEK-EVAALEK-EVAALEK-EVAALEK), whose glutamate residues will form a negative charge at pH 7, while the other of the Heterodimer-Promoting Domains will comprise four tandem “K-coil” domains (SEQ ID NO:22: KVAALKE-KVAALKE-KVAALKE-KVAALKE), whose lysine residues will form a positive charge at pH 7. The presence of such charged domains promotes association between the first and second polypeptides, and thus fosters heterodimerization. Especially preferred is a Heterodimer-Promoting Domain in which one of the four tandem “E-coil” helical domains of SEQ ID NO:21 has been modified to contain a cysteine residue: EVAACEK-EVAALEK-EVAALEK-EVAALEK (SEQ ID NO:23). Likewise, especially preferred is a Heterodimer-Promoting Domain in which one of the four tandem “K-coil” helical domains of SEQ ID NO:22 has been modified to contain a cysteine residue: KVAACKE-KVAALKE-KVAALKE-KVAALKE (SEQ ID NO:24).

2. Fc-Domain-Containing Bispecific Diabodies

In a further preferred embodiment, the CD32B x CD79B diabodies of the present invention additionally comprise an Fc Domain. The Fc Domain of such Fc Domain-containing diabodies of the present invention may be either a complete Fc Region (e.g., a complete IgG Fc Region) or only a fragment of a complete Fc Region. Although the Fc Domain of the bispecific monovalent Fc diabodies of the present invention may possess the ability to bind to one or more Fc receptors (e.g., FcγR(s)), more preferably such Fc Domain will have substantially reduced or no ability to bind to FcγRIA (CD64), FcγRIIA (CD32A), FcγRIIB (CD32B), FcγRIIIA (CD16a) or FcγRIIIB (CD16b) (relative to the binding exhibited by a wild-type Fc Region). The Fc Domain of the bispecific monovalent Fc diabodies of the present invention may include some or all of the CH2 Domain and/or some or all of the CH3 Domain of a complete Fc Region, or may comprise a variant CH2 and/or a variant CH3 sequence (that may include, for example, one or more insertions and/or one or more deletions with respect to the CH2 or CH3 Domains of a complete Fc Region). The Fc Domain of the bispecific monovalent Fc diabodies of the present invention may comprise non-Fc polypeptide portions, or may comprise portions of non-naturally complete Fc regions, or may comprise non-naturally occurring orientations of CH2 and/or CH3 Domains (such as, for example, two CH2 Domains or two CH3 Domains, or in the N-terminal to C-terminal direction, a CH3 Domain linked to a CH2 Domain, etc.).

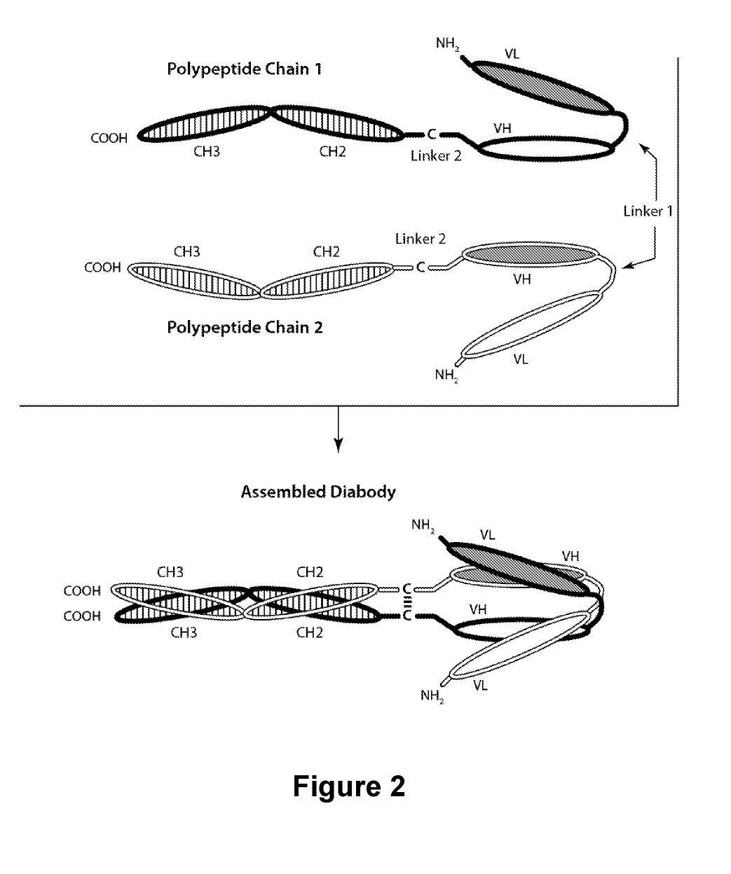

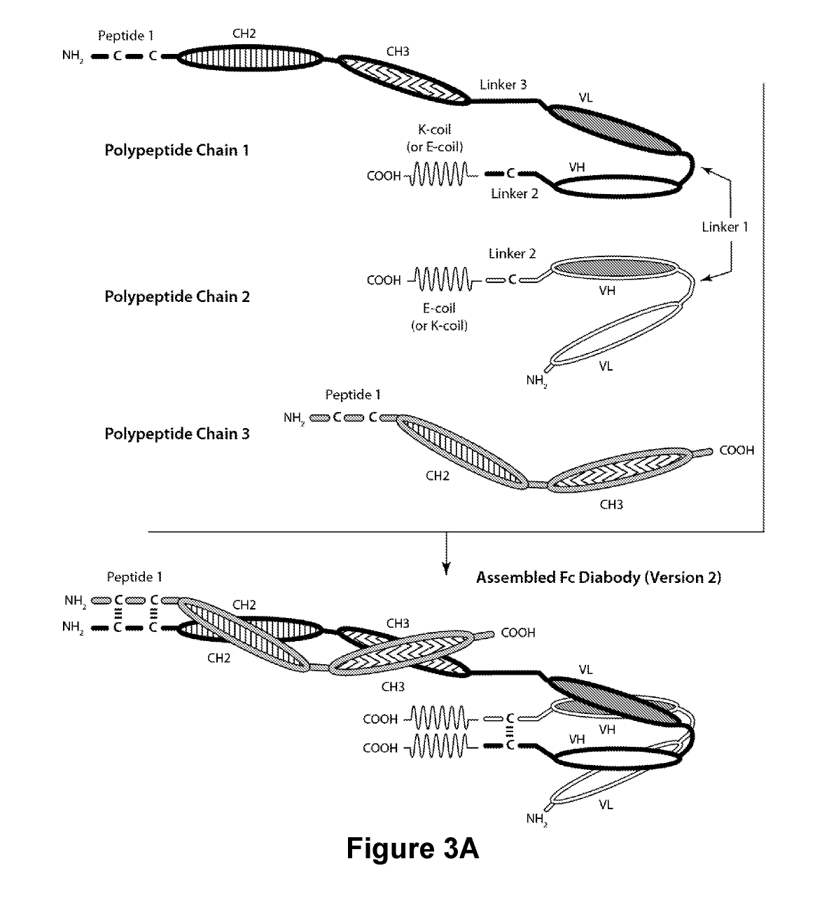

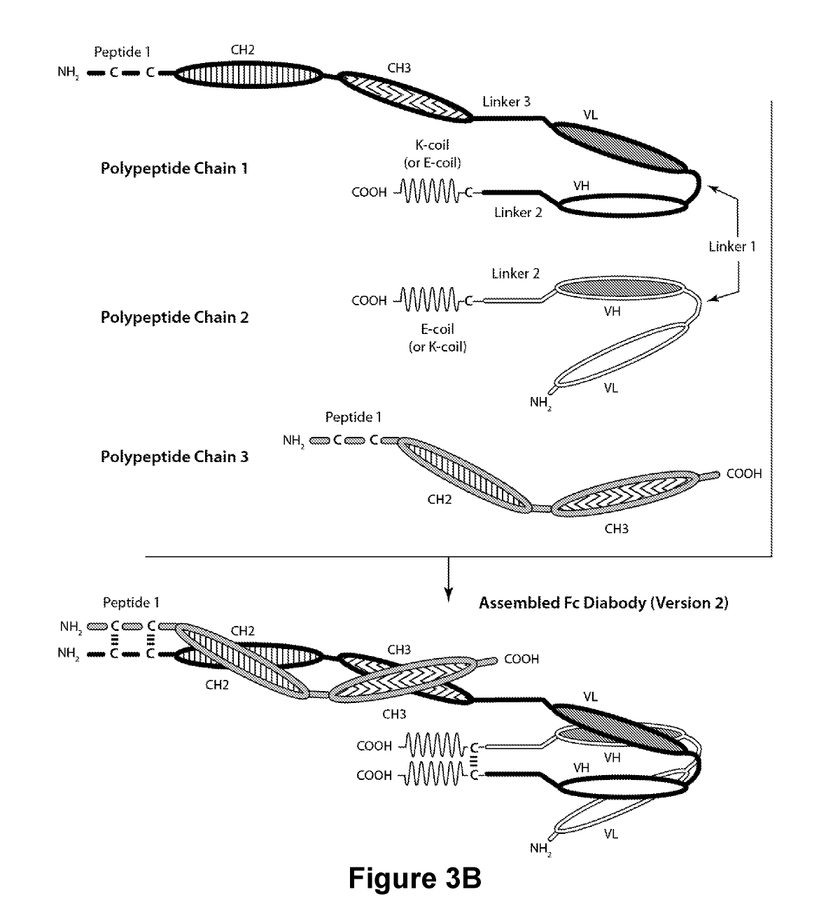

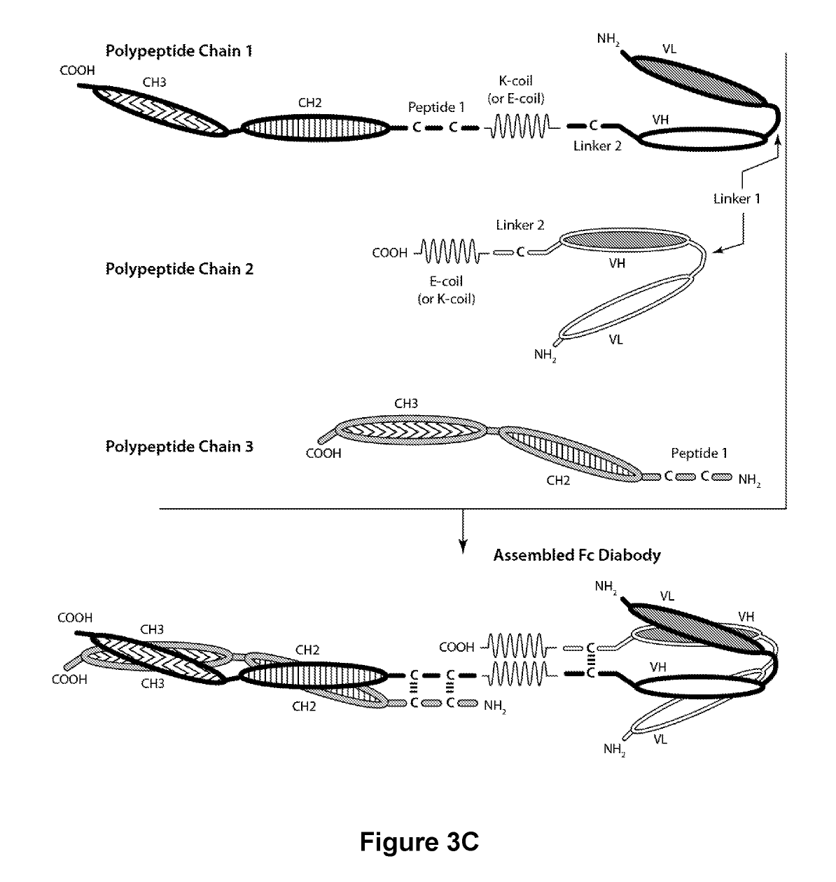

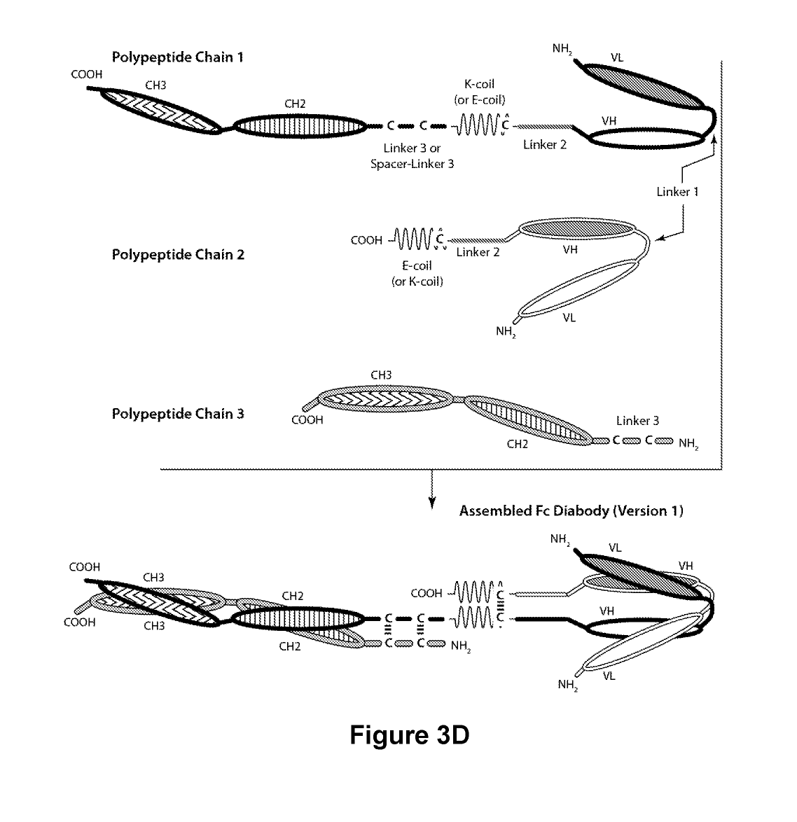

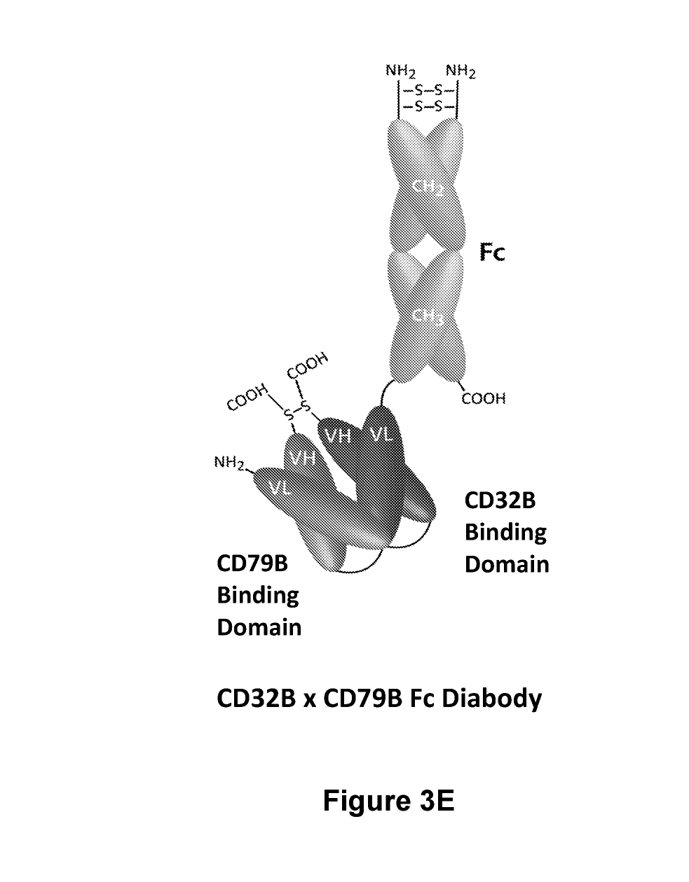

Such Fc-Domain-containing diabodies of the present invention may comprise two polypeptide chains (e.g., FIG. 2), or may comprise three (e.g., FIGS. 3A-3E) or more polypeptide chains. FIG. 2 shows a diabody having a structure similar to that described above, except that the respective Heterodimer-Promoting Domains are replaced with CH2-CH3 Domains. Preferably, the Fc Domain formed by such polypeptide chains chain will have substantially reduced or no ability to bind to activating FcγR, such as FcγRIA (CD64), FcγRIIA (CD32A), FcγRIIIA (CD16a) or FcγRIIIB (CD16b) (relative to the binding exhibited by a wild-type Fc Region).

FIGS. 3A-3E show alternative CD32B x CD79B Fc diabodies composed of three polypeptide chains, of which the first and second polypeptide chains are covalently bonded to one another and the first and third polypeptide chains are covalently bonded to one another. As in the above-described diabodies, the VL Domain of the first polypeptide chain interacts with the VH Domain of the second polypeptide chain in order to form a first functional antigen binding site that is specific for the first antigen (i.e., either CD32B or CD79B). Likewise, the VL Domain of the second polypeptide chain interacts with the VH Domain of the first polypeptide chain in order to form a second functional antigen binding site that is specific for the second antigen (i.e., either CD79B or CD32B, depending upon the identity of the first antigen). Thus, the selection of the VL and VH Domains of the first and second polypeptide chains are coordinated, such that the two polypeptide chains collectively comprise VL and VH Domains capable of binding to CD32B and CD79B (i.e., they comprise VL_{CD32B}/VH_{CD32B} and VL_{CD79B}/VH_{CD79B}). Collectively, each such VL and VH Domain, and the intervening Linker that separates them, are referred to as an Antigen-Binding Domain of the molecule.

In the CD32B x CD79B bispecific Fc diabody embodiment shown in FIG. 3A and FIG. 3B, the first polypeptide chain comprises (in the N-terminal to C-terminal direction): an amino terminus, a cysteine-containing peptide (Peptide 1), an IgG Fc Domain composed of all or part of the CH2 and CH3 Domains of an antibody Fc Region, an intervening linker peptide (Linker 3), the VL Domain of a monoclonal antibody capable of binding to either CD32B or CD79B (i.e., either VL_{CD32B} or VL_{CD79B}), an intervening peptide (Linker 1), a VH Domain of a monoclonal antibody capable of binding to either CD79B (if such first polypeptide chain contains VL_{CD32B}) or CD32B (if such first polypeptide chain contains VL_{CD79B}), an intervening spacer peptide (Linker 2), a Heterodimer-Promoting Domain, an optional fourth spacer peptide (Linker 4) to provide improved stabilization to the Heterodimer-Promoting Domain and a C-terminus.

The second polypeptide chain of such CD32B x CD79B bispecific Fc diabody embodiments comprises (in the N-terminal to C-terminal direction): an amino terminus, a VL Domain of a monoclonal antibody capable of binding to either CD79B or CD32B (i.e., either VL_{CD79B} or VL_{CD32B}, depending upon the VL Domain selected for the first polypeptide chain of the diabody), an intervening linker peptide (Linker 1), a VH Domain of a monoclonal antibody capable of binding to either CD32B (if such second polypeptide chain contains VL_{CD79B}) or CD32B (if such second polypeptide chain contains VL_{CD32B}), an intervening spacer peptide (Linker 2), a Heterodimer-Promoting Domain, and a C-terminus.

The third polypeptide chain of such preferred CD32B x CD79B bispecific Fc diabody comprises (in the N-terminal to C-terminal direction): an amino terminus, a cysteine-containing peptide (Peptide 1), an IgG Fc Domain (preferably, the CH2 and CH3 Domains of an antibody Fc Region) having the same isotype as that of the Fc Domain of the first polypeptide chain and a C-terminus.

The embodiment of the CD32B x CD79B bispecific Fc diabody shown in FIG. 3A, differs from that shown in FIG. 3B in that the intervening spacer peptide (Linker 2) of the first and second polypeptide chains do not contain a cysteine residue, such cysteine residue now being part of the Heterodimer-Promoting Domains of these polypeptide chains.

In the CD32B x CD79B bispecific Fc diabody embodiment shown in FIG. 3C, the first polypeptide chain comprises (in the N-terminal to C-terminal direction): an amino terminus, the VL Domain of a monoclonal antibody capable of binding to either CD32B or CD79B (i.e., either VL_{CD32B} or VL_{CD79B}), an intervening peptide (Linker 1), a VH Domain of a monoclonal antibody capable of binding to either CD79B (if such first polypeptide chain contains VL_{CD32B}) or CD32B (if such first polypeptide chain contains VL_{CD79B}), a cysteine-containing intervening spacer peptide (Linker 2), a Heterodimer-Promoting Domain, a cysteine-containing peptide (Peptide 1), an IgG Fc Domain composed of all or part of the CH2 and CH3 Domains of an antibody Fc Region, and a C-terminus.

The second polypeptide chain of such CD32B x CD79B bispecific Fc diabody embodiments comprises (in the N-terminal to C-terminal direction): an amino terminus, a VL Domain of a monoclonal antibody capable of binding to either CD79B or CD32B (i.e., either VL_{CD79B} or VL_{CD32B}, depending upon the VL Domain selected for the first polypeptide chain of the diabody), an intervening linker peptide (Linker 1), a VH Domain of a monoclonal antibody capable of binding to either CD32B (if such second polypeptide chain contains VL_{CD79B}) or CD32B (if such second polypeptide chain contains VL_{CD32B}), a cysteine-containing intervening spacer peptide (Linker 2), a Heterodimer-Promoting Domain, and a C-terminus.

The third polypeptide chain of the CD32B x CD79B bispecific Fc diabody of FIG. 3C and FIG. 3D comprise (in the N-terminal to C-terminal direction): an amino terminus, a cysteine-containing peptide (Peptide 1), an IgG Fc Domain (preferably, the CH2 and CH3 Domains of an antibody Fc Region) having the same isotype as that of the Fc Domain of the first polypeptide chain and a C-terminus.

The embodiment of the CD32B x CD79B bispecific Fc diabody shown in FIG. 3D, differs from that shown in FIG. 3C in that the intervening spacer peptide (Linker 2) of the first and second polypeptide chains do not contain a cysteine residue, such cysteine residue now being part of the Heterodimer-Promoting Domains of these polypeptide chains.

The cysteine-containing peptide (Peptide 1) of the first and third polypeptide chains may be comprised of the same amino acid sequence or of different amino acid sequences, and will contain 1, 2, 3 or more cysteine residues. A particularly preferred Peptide 1 has the amino acid sequence (SEQ ID NO:25): DKTHTCPPCP or (SEQ ID NO:26) GGGDKTHTCPPCP. A preferred intervening linker peptide (Linker 3) comprises the amino acid sequence (SEQ ID NO:27): APSSS, and more preferably has the amino acid sequence (SEQ ID NO:28): APSSSPME. A preferred fourth spacer peptide (Linker 4) has the sequence GGG or is SEQ ID NO:29: GGGNS.

Preferably, the Fc Domain formed by the first and third polypeptide chains of the Fc-containing diabodies of the invention have substantially reduced or no ability to bind to activating FcγR, such as FcγRIA (CD64), FcγRIIA (CD32A), FcγRIIIA (CD16a) or FcγRIBB (CD16b) (relative to the binding exhibited by a wild-type Fc Region). Fc Domains having mutations that reduce or eliminate binding to such receptors are well known in the art and include amino acid substitutions at positions 234 and 235, a substitution at position 265 or a substitution at position 297 (see, for example, U.S. Pat. No. 5,624,821, herein incorporated by reference). In a preferred embodiment, the CH2 and CH3 Domain includes a substitution at position 234 with alanine and 235 with alanine.

The CH2 and/or CH3 Domains of the first and third polypeptide chains of the Fc-containing diabodies of the invention need not be identical, and advantageously are modified to foster complexing between the two polypeptides. For example, an amino acid substitution (preferably a substitution with an amino acid comprising a bulky side group forming a ‘knob’, e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., ‘the hole’ (e.g., a substitution with glycine). Such sets of mutations can be engineered into any pair of polypeptides comprising the Fc diabody molecule, and further, engineered into any portion of the polypeptides chains of said pair. Methods of protein engineering to favor heterodimerization over homodimerization are well known in the art, in particular with respect to the engineering of immunoglobulin-like molecules, and are encompassed herein (see e.g., Ridgway et al. (1996) “Knobs-Into-Holes'Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270: 26-35, and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety). Preferably the ‘knob’ is engineered into the CH2-CH3 Domains of the first polypeptide chain and the ‘hole’ is engineered into the CH2-CH3 Domains of the third polypeptide chain. Thus, the ‘knob’ will help in preventing the first polypeptide chain from homodimerizing via its CH2 and/or CH3 Domains. As the third polypeptide chain preferably contains the ‘hole’ substitution it will heterodimerize with the first polypeptide chain as well as homodimerize with itself. A preferred knob is created by modifying a native IgG Fc Region to contain the modification T366W. A preferred hole is created by modifying a native IgG Fc Region to contain the modification T366S, L368A and Y407V. To aid in purifying the third polypeptide chain homodimer from the final bispecific monovalent Fc diabody comprising the first, second and third polypeptide chains, the protein A binding site of the CH2 and CH3 Domains of the third polypeptide chain is preferably mutated by amino acid substitution at position 435 (H435R). To aid in purifying the third polypeptide chain homodimer from the final bispecific monovalent Fc diabody comprising the first, second and third polypeptide chains, the protein A binding site of the CH2 and CH3 Domains of the third polypeptide chain is preferably mutated by amino acid substitution. Thus, the third polypeptide chain homodimer will not bind to protein A, whereas the bispecific monovalent Fc diabody will retain its ability to bind protein A via the protein A binding site on the first polypeptide chain.

3. Exemplary CD32B x CD79B Bispecific Diabodies

An exemplary CD32B x CD79B bispecific diabody of the invention will comprise two or more polypeptide chains, and will comprise:
(1) a VL Domain of an antibody that binds CD32B (VL_{CD32B}), such VL_{CD32B} Domain having the sequence (SEQ ID NO:30):

DIQMTQSPSS LSASVGDRVT ITCRASQEIS GYLSWLQQKP
GKAPRRLIYA ASTLDSGVPS RFSGSESGTE FTLTISSLQP
EDFATYYCLQ YFSYPLTFGG GTKVEIK

(2) A VH Domain of an antibody that binds CD32B (VH_{CD32B}), such VH_{CD32B} Domain having the sequence (SEQ ID NO:31):

EVQLVESGGG LVQPGGSLRL SCAASGFTFS DAWMDWVRQA
PGKGLEWVAE IRNKAKNHAT YYAESVIGRF TISRDDAKNS
LYLQMNSLRA EDTAVYYCGA LGLDYWGQGT LVTVSS

(3) A VL Domain of an antibody that binds CD79B (VL_{CD79B}), such VL_{CD79B} Domain having the sequence (SEQ ID NO:32):

DVVMTQSPLS LPVTLGQPAS ISCKSSQSLL DSDGKTYLNW
FQQRPGQSPN RLIYLVSKLD SGVPDRFSGS GSGTDFTLKI
SRVEAEDVGV YYCWQGTHFP LTFGGGTKLE IK

(4) A VH Domain of an antibody that binds CD79B (VH_{CD79B}), such VH_{CD79B} Domain having the sequence (SEQ ID NO:33):

QVQLVQSGAE VKKPGASVKV SCKASGYTFT SYWMNWVRQA
PGQGLEWIGM IDPSDSETHY NQKFKDRVTM TTDTSTSTAY
MELRSLRSDD TAVYYCARAM GYWGQGTTVT VSS

A first exemplary CD32B x CD79B bispecific diabody of the invention has two polypeptide chains. The first polypeptide chain of such exemplary diabody has the structure, in the N-terminal to C-terminal direction, of: an N-terminus, the above-indicated VL_{CD32B} Domain, a Linker 1, the above-indicated VH_{C}D79B Domain, a cysteine-containing Linker 2, an E-coil Domain, and a C-terminus. The amino acid sequence of such a preferred polypeptide is (SEQ ID NO:34):

DIQMTQSPSS LSASVGDRVT ITCRASQEIS GYLSWLQQKP
GKAPRRLIYA ASTLDSGVPS RFSGSESGTE FTLTISSLQP
EDFATYYCLQ YFSYPLTFGG GTKVEIKGGG SGGGGQVQLV
QSGAEVKKPG ASVKVSCKAS GYTFTSYWMN WVRQAPGQGL
EWIGMIDPSD SETHYNQKFK DRVTMTTDTS TSTAYMELRS
LRSDDTAVYY CARAMGYWGQ GTTVTVSSGG CGGGEVAALE
KEVAALEKEV AALEKEVAAL EK

In SEQ ID NO:34, amino acid residues 1-107 are the VL Domain of an antibody that binds CD32B (VL_{CD32B}) (SEQ ID NO:30), amino acid residues 108-115 are Linker 1 (SEQ ID NO:14), amino acid residues 116-228 is the VH Domain of an antibody that binds CD79B (VH_{CD79B}) (SEQ ID NO:33), amino acid residues 229-234 are the cysteine-containing Linker 2 (SEQ ID NO:16), amino acid residues 235-262 are the heterodimer-promoting E-coil Domain (SEQ ID NO:21).

The second polypeptide chain of such exemplary diabody has the amino acid sequence, in the N-terminal to C-terminal direction, of (SEQ ID NO:35):

DVVMTQSPLS LPVTLGQPAS ISCKSSQSLL DSDGKTYLNW
FQQRPGQSPN RLIYLVSKLD SGVPDRFSGS GSGTDFTLKI
SRVEAEDVGV YYCWQGTHFP LTFGGGTKLE IKGGGSGGGG
EVQLVESGGG LVQPGGSLRL SCAASGFTFS DAWMDWVRQA
PGKGLEWVAE IRNKAKNHAT YYAESVIGRF TISRDDAKNS
LYLQMNSLRA EDTAVYYCGA LGLDYWGQGT LVTVSSGGCG
GGKVAALKEK VAALKEKVAA LKEKVAALKE

In SEQ ID NO:35, amino acid residues 1-112 is the VL Domain of an antibody that binds CD79B (VL_{CD79B}) (SEQ ID NO:32), amino acid residues 113-120 are Linker 1 (SEQ ID NO:14), amino acid residues 121-236 is the VH Domain of an antibody that binds CD32B (VH_{CD32B}) (SEQ ID NO:31), amino acid residues 237-242 are a cysteine-containing Linker 2 (SEQ ID NO:16), and amino acid residues 243-270 are the heterodimer-promoting K-coil Domain (SEQ ID NO:22).

A second exemplary CD32B x CD79B bispecific diabody of the invention has two polypeptide chains, in which the first polypeptide chain has the structure, in the N-terminal to C-terminal direction, of: an N-terminus, the above-indicated VL_{C}D32B Domain, a Linker 1, the above-indicated VH_{C}D79B Domain, a Linker 2, a cysteine-containing E-coil Domain, and a C-terminus. The amino acid sequence of such a preferred polypeptide is (SEQ ID NO:36):

DIQMTQSPSS LSASVGDRVT ITCRASQEIS GYLSWLQQKP
GKAPRRLIYA ASTLDSGVPS RFSGSESGTE FTLTISSLQP
EDFATYYCLQ YFSYPLTFGG GTKVEIKGGG SGGGGQVQLV
QSGAEVKKPG ASVKVSCKAS GYTFTSYWMN WVRQAPGQGL
EWIGMIDPSD SETHYNQKFK DRVTMTTDTS TSTAYMELRS
LRSDDTAVYY CARAMGYWGQ GTTVTVSSAS TKGEVAACEK
EVAALEKEVA ALEKEVAALE K

In SEQ ID NO:36, amino acid residues 1-107 are the VL Domain of an antibody that binds CD32B (VL_{CD32B}) (SEQ ID NO:30), amino acid residues 108-115 are Linker 1 (SEQ ID NO:14), amino acid residues 116-228 is the VH Domain of an antibody that binds CD79B (VH_{CD79B}) (SEQ ID NO:33), amino acid residues 229-233 are Linker 2 (SEQ ID NO:15), amino acid residues 234-261 are the cysteine-containing heterodimer-promoting E-coil Domain (SEQ ID NO:23).

The second polypeptide chain of such second exemplary diabody has the amino acid sequence, in the N-terminal to C-terminal direction, of (SEQ ID NO:37):

DVVMTQSPLS LPVTLGQPAS ISCKSSQSLL DSDGKTYLNW
FQQRPGQSPN RLIYLVSKLD SGVPDRFSGS GSGTDFTLKI
SRVEAEDVGV YYCWQGTHFP LTFGGGTKLE IKGGGSGGGG
EVQLVESGGG LVQPGGSLRL SCAASGFTFS DAWMDWVRQA
PGKGLEWVAE IRNKAKNHAT YYAESVIGRF TISRDDAKNS
LYLQMNSLRA EDTAVYYCGA LGLDYWGQGT LVTVSSASTK
GKVAACKEKV AALKEKVAAL KEKVAALKE

In SEQ ID NO:37, amino acid residues 1-112 is the VL Domain of an antibody that binds CD79B (VL_{CD79B}) (SEQ ID NO:32), amino acid residues 113-120 are Linker 1 (SEQ ID NO:14), amino acid residues 121-236 is the VH Domain of an antibody that binds CD32B (VH_{CD32B}) (SEQ ID NO:31), amino acid residues 237-241 are Linker 2 (SEQ ID NO:15), and amino acid residues 242-269 are the cysteine-containing heterodimer-promoting K-coil Domain (SEQ ID NO:24).

4. Exemplary CD32B x CD79B Bispecific Fc Diabodies

A first exemplary CD32B x CD79B bispecific Fc diabody of the invention has three polypeptide chains (FIG. 3A). The first polypeptide chain will comprise CH2 and CH3 Domains of a knob-containing IgG Fc Region having the sequence (SEQ ID NO:38):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGK

Thus, the first polypeptide chain of such exemplary Fc diabody has the structure, in the N-terminal to C-terminal direction, of: Peptide 1, a CH2-CH3 Domain of an IgG Fc Region, Linker 1, a VL Domain of an antibody that binds CD32B (VL_{CD32B}), a cysteine-containing Linker 2, a VH Domain of an antibody that binds CD79B (VH_{CD79B}), Linker 3, an E-coil Domain, a Linker 4 and a C-terminus. The amino acid sequence of such a preferred polypeptide is (SEQ ID NO:39):

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLWCLVK GFYPSDIAVE
WESNGQPENN YKTTPPVLDS DGSFFLYSKL TVDKSRWQQG
NVFSCSVMHE ALHNHYTQKS LSLSPGKAPS SSPMEDIQMT
QSPSSLSASV GDRVTITCRA SQEISGYLSW LQQKPGKAPR
RLIYAASTLD SGVPSRFSGS ESGTEFTLTI SSLQPEDFAT
YYCLQYFSYP LTFGGGTKVE IKGGGSGGGG QVQLVQSGAE
VKKPGASVKV SCKASGYTFT SYWMNWVRQA PGQGLEWIGM
IDPSDSETHY NQKFKDRVTM TTDTSTSTAY MELRSLRSDD
TAVYYCARAM GYWGQGTTVT VSSGGCGGGE VAALEKEVAA
LEKEVAALEK EVAALEKGGG NS

In SEQ ID NO:39, amino acid residues 1-10 are Peptide 1 (SEQ ID NO:25), amino acid residues 11-227 are the CH2 and CH3 Domains of a knob-containing IgG Fc Region (SEQ ID NO:38), amino acid residues 228-235 are Linker 3 (SEQ ID NO:28), amino acid residues 236-342 is the VL Domain of an antibody that binds CD32B (VL_{CD32B}) (SEQ ID NO:30), amino acid residues 343-350 are Linker 1 (SEQ ID NO:14), amino acid residues 351-463 is the VH Domain of an antibody that binds CD79B (VH_{CD79B}) (SEQ ID NO:33), amino acid residues 464-469 are a cysteine-containing Linker 2 (SEQ ID NO:16), amino acid residues 470-497 are the heterodimer-promoting E-coil Domain (SEQ ID NO:21), and amino acid residues 498-502 are Linker 4 (SEQ ID NO:29).

A preferred polynucleotide that encodes the first polypeptide chain has the sequence (SEQ ID NO:40):

gacaaaactc acacatgccc accgtgccca gcacctgaag
ccgcgggggg accgtcagtc ttcctcttcc ccccaaaacc
caaggacacc ctcatgatct cccggacccc tgaggtcaca
tgcgtggtgg tggacgtgag ccacgaagac cctgaggtca
agttcaactg gtacgtggac ggcgtggagg tgcataatgc
caagacaaag ccgcgggagg agcagtacaa cagcacgtac
cgtgtggtca gcgtcctcac cgtcctgcac caggactggc
tgaatggcaa ggagtacaag tgcaaggtct ccaacaaagc
cctcccagcc cccatcgaga aaaccatctc caaagccaaa
gggcagcccc gagaaccaca ggtgtacacc ctgcccccat
cccgggagga gatgaccaag aaccaggtca gcctgtggtg
cctggtcaaa ggcttctatc ccagcgacat cgccgtggag
tgggagagca atgggcagcc ggagaacaac tacaagacca
cgcctcccgt gctggactcc gacggctcct tcttcctcta
cagcaagctc accgtggaca agagcaggtg gcagcagggg
aacgtcttct catgctccgt gatgcatgag gctctgcaca
accactacac gcagaagagc ctctccctgt ctccgggtaa
agccccttcc agctccccta tggaagacat ccagatgacc
cagtctccat cctccttatc tgcctctgtg ggagatagag
tcaccatcac ttgtcgggca agtcaggaaa ttagtggtta
cttaagctgg ctgcagcaga aaccaggcaa ggcccctaga
cgcctgatct acgccgcatc cactttagat tctggtgtcc
catccaggtt cagtggcagt gagtctggga ccgagttcac
cctcaccatc agcagccttc agcctgaaga ttttgcaacc
tattactgtc tacaatattt tagttatccg ctcacgttcg
gaggggggac caaggtggaa ataaaaggag gcggatccgg
cggcggaggc caggttcagc tggtgcagtc tggagctgag
gtgaagaagc ctggcgcctc agtgaaggtc tcctgcaagg
cttctggtta cacctttacc agctactgga tgaactgggt
gcgacaggcc cctggacaag ggcttgagtg gatcggaatg
attgatcctt cagacagtga aactcactac aatcaaaagt
tcaaggacag agtcaccatg accacagaca catccacgag
cacagcctac atggagctga ggagcctgag atctgacgac
acggccgtgt attactgtgc gagagctatg ggctactggg
ggcaagggac cacggtcacc gtctcctccg gaggatgtgg
cggtggagaa gtggccgcac tggagaaaga ggttgctgct
ttggagaagg aggtcgctgc acttgaaaag gaggtcgcag
ccctggagaa aggcggcggg aactct

The second polypeptide chain of such exemplary Fc diabody has the structure, in the N-terminal to C-terminal direction, of: VL Domain of an antibody that binds CD79B (VL_{CD79B}), Linker 1, VH Domain of an antibody that binds CD32B (VH_{CD32B}), a cysteine-containing Linker 2, the heterodimer-promoting K-coil Domain, and a C-terminus.

A preferred sequence for the second polypeptide chain is (SEQ ID NO:41):

DVVMTQSPLS LPVTLGQPAS ISCKSSQSLL DSDGKTYLNW
FQQRPGQSPN RLIYLVSKLD SGVPDRFSGS GSGTDFTLKI
SRVEAEDVGV YYCWQGTHFP LTFGGGTKLE IKGGGSGGGG
EVQLVESGGG LVQPGGSLRL SCAASGFTFS DAWMDWVRQA
PGKGLEWVAE IRNKAKNHAT YYAESVIGRF TISRDDAKNS
LYLQMNSLRA EDTAVYYCGA LGLDYWGQGT LVTVSSGGCG
GGKVAALKEK VAALKEKVAA LKEKVAALKE

In SEQ ID NO:41, amino acid residues 1-112 is the VL Domain of an antibody that binds CD79B (VL_{CD79B}) (SEQ ID NO:32), amino acid residues 113-120 are Linker 1 (SEQ ID NO:14), amino acid residues 121-236 is the VH Domain of an antibody that binds CD32B (VH_{CD32B}) (SEQ ID NO:31), amino acid residues 237-242 are the cysteine-containing Linker 2 (SEQ ID NO:16), and amino acid residues 243-270 are the heterodimer-promoting K-coil Domain (SEQ ID NO:22).

A preferred polynucleotide that encodes the second polypeptide chain has the sequence (SEQ ID NO:42):

gatgttgtga tgactcagtc tccactctcc ctgcccgtca
cccttggaca gccggcctcc atctcctgca agtcaagtca
gagcctctta gatagtgatg gaaagacata tttgaattgg
tttcagcaga ggccaggcca atctccaaac cgcctaattt
atctggtgtc taaactggac tctggggtcc cagacagatt
cagcggcagt gggtcaggca ctgatttcac actgaaaatc
agcagggtgg aggctgagga tgttggggtt tattactgct
ggcaaggtac acattttccg ctcacgttcg gcggagggac
caagcttgag atcaaaggag gcggatccgg cggcggaggc
gaagtgcagc ttgtggagtc tggaggaggc ttggtgcaac
ctggaggatc cctgagactc tcttgtgccg cctctggatt
cacttttagt gacgcctgga tggactgggt ccgtcaggcc
ccaggcaagg ggcttgagtg ggttgctgaa attagaaaca
aagctaaaaa tcatgcaaca tactatgctg agtctgtgat
agggaggttc accatctcaa gagatgacgc caaaaacagt
ctgtacctgc aaatgaacag cttaagagct gaagacactg
ccgtgtatta ctgtggggct ctgggccttg actactgggg
ccaaggcacc ctggtgaccg tctcctccgg aggatgtggc
ggtggaaaag tggccgcact gaaggagaaa gttgctgctt
tgaaagagaa ggtcgccgca cttaaggaaa aggtcgcagc
cctgaaagag

Such exemplary CD32B x CD79B bispecific Fc diabody will have a third polypeptide chain that will comprise CH2 and CH3 Domains of a hole-containing IgG Fc Region having the amino acid sequence (SEQ ID NO:43):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGK

Thus, the amino acid sequence of the third polypeptide chain of such exemplary CD32B x CD79B bispecific Fc diabody is SEQ ID NO:44:

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GFYPSDIAVE
WESNGQPENN YKTTPPVLDS DGSFFLVSKL TVDKSRWQQG
NVFSCSVMHE ALHNRYTQKS LSLSPGK

In SEQ ID NO:44, amino acid residues 1-10 are Peptide 1 (SEQ ID NO:25), and amino acid residues 11-227 are the CH2 and CH3 Domains of a hole-containing IgG Fc Region (SEQ ID NO:43).

A preferred polynucleotide that encodes the third polypeptide chain has the sequence (SEQ ID NO:45):

gacaaaactc acacatgccc accgtgccca gcacctgaag
ccgcgggggg accgtcagtc ttcctcttcc ccccaaaacc
caaggacacc ctcatgatct cccggacccc tgaggtcaca
tgcgtggtgg tggacgtgag ccacgaagac cctgaggtca
agttcaactg gtacgtggac ggcgtggagg tgcataatgc
caagacaaag ccgcgggagg agcagtacaa cagcacgtac
cgtgtggtca gcgtcctcac cgtcctgcac caggactggc
tgaatggcaa ggagtacaag tgcaaggtct ccaacaaagc
cctcccagcc cccatcgaga aaaccatctc caaagccaaa
gggcagcccc gagaaccaca ggtgtacacc ctgcccccat
cccgggagga gatgaccaag aaccaggtca gcctgagttg
cgcagtcaaa ggcttctatc ccagcgacat cgccgtggag
tgggagagca atgggcagcc ggagaacaac tacaagacca
cgcctcccgt gctggactcc gacggctcct tcttcctcgt
cagcaagctc accgtggaca agagcaggtg gcagcagggg
aacgtcttct catgctccgt gatgcatgag gctctgcaca
accgctacac gcagaagagc ctctccctgt ctccgggtaa a

A second exemplary CD32B x CD79B bispecific Fc diabody of the invention also has three polypeptide chains (FIG. 3B). The first polypeptide chain comprises CH2 and CH3 Domains of a knob-containing IgG Fc Region having the amino acid sequence of SEQ ID NO:38.

Thus, the first polypeptide chain of such second exemplary Fc diabody has the structure, in the N-terminal to C-terminal direction, of: Peptide 1, a CH2-CH3 Domain of a knob-containing IgG Fc Region, Linker 1, a VL Domain of an antibody that binds CD32B (VL_{CD32B}), Linker 2, a VH Domain of an antibody that binds CD79B (VH_{CD79B}), Linker 3, a cysteine-containing E-coil Domain, a Linker 4 and a C-terminus. The amino acid sequence of such a preferred polypeptide is (SEQ ID NO:46):

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLWCLVK GFYPSDIAVE
WESNGQPENN YKTTPPVLDS DGSFFLYSKL TVDKSRWQQG
NVFSCSVMHE ALHNHYTQKS LSLSPGKAPS SSPMEDIQMT
QSPSSLSASV GDRVTITCRA SQEISGYLSW LQQKPGKAPR
RLIYAASTLD SGVPSRFSGS ESGTEFTLTI SSLQPEDFAT
YYCLQYFSYP LTFGGGTKVE IKGGGSGGGG QVQLVQSGAE
VKKPGASVKV SCKASGYTFT SYWMNWVRQA PGQGLEWIGM
IDPSDSETHY NQKFKDRVTM TTDTSTSTAY MELRSLRSDD
TAVYYCARAM GYWGQGTTVT VSSASTKGEV AACEKEVAAL
EKEVAALEKE VAALEKGGGN S

In SEQ ID NO:46, amino acid residues 1-10 are Peptide 1 (SEQ ID NO:25), amino acid residues 11-227 are the CH2 and CH3 Domains of a knob-containing IgG Fc Region (SEQ ID NO:38), amino acid residues 228-235 are Linker 3 (SEQ ID NO:28), amino acid residues 236-342 is the VL Domain of an antibody that binds CD32B (VL_{CD32B}) (SEQ ID NO:30), amino acid residues 343-350 are Linker 1 (SEQ ID NO:14), amino acid residues 351-463 is the VH Domain of an antibody that binds CD79B (VH_{CD79B}) (SEQ ID NO:33), amino acid residues 464-468 are Linker 2 (SEQ ID NO:15), amino acid residues 469-496 are the cysteine-containing heterodimer-promoting E-coil Domain (SEQ ID NO:23), and amino acid residues 497-501 are Linker 4 (SEQ ID NO:29).

The second polypeptide chain of such second exemplary Fc diabody has the structure, in the N-terminal to C-terminal direction, of: VL Domain of an antibody that binds CD79B (VL_{CD79B}), Linker 1, VH Domain of an antibody that binds CD32B (VH_{CD32B}), Linker 2, a cysteine-containing heterodimer-promoting K-coil Domain, and a C-terminus.

A preferred sequence for the second polypeptide chain is (SEQ ID NO:47):

DVVMTQSPLS LPVTLGQPAS ISCKSSQSLL DSDGKTYLNW
FQQRPGQSPN RLIYLVSKLD SGVPDRFSGS GSGTDFTLKI
SRVEAEDVGV YYCWQGTHFP LTFGGGTKLE IKGGGSGGGG
EVQLVESGGG LVQPGGSLRL SCAASGFTFS DAWMDWVRQA
PGKGLEWVAE IRNKAKNHAT YYAESVIGRF TISRDDAKNS
LYLQMNSLRA EDTAVYYCGA LGLDYWGQGT LVTVSSASTK
GKVAACKEKV AALKEKVAAL KEKVAALKE

In SEQ ID NO:47, amino acid residues 1-112 is the VL Domain of an antibody that binds CD79B (VL_{CD79B}) (SEQ ID NO:32), amino acid residues 113-120 are Linker 1 (SEQ ID NO:14), amino acid residues 121-236 is the VH Domain of an antibody that binds CD32B (VH_{CD32B}) (SEQ ID NO:31), amino acid residues 237-241 are Linker 2 (SEQ ID NO:15), and amino acid residues 242-269 are the cysteine-containing heterodimer-promoting K-coil Domain (SEQ ID NO:24).

The third polypeptide chain of such second exemplary Fc diabody will comprise CH2 and CH3 Domains of a hole-containing IgG region (SEQ ID NO:43).

Thus, the amino acid sequence of the third polypeptide chain of such exemplary CD32B x CD79B bispecific Fc diabody is SEQ ID NO:48:

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GFYPSDIAVE
WESNGQPENN YKTTPPVLDS DGSFFLVSKL TVDKSRWQQG
NVFSCSVMHE ALHNRYTQKS LSLSPGK

In SEQ ID NO:48, amino acid residues 1-10 are Peptide 1 (SEQ ID NO:25), and amino acid residues 11-227 are the CH2 and CH3 Domains of a hole-containing IgG Fc Region (SEQ ID NO:43).

An alternative CD32B x CD79B bispecific monovalent Fc diabody molecule of the present invention is shown schematically in FIG. 3C. Such alternative CD32B x CD79B Fc diabody molecules possess three polypeptide chains, of which the first and second polypeptide chains are covalently bonded to one another and the first and third polypeptide chains are bonded to one another. The alternative CD32B x CD79B bispecific monovalent Fc diabody molecules differ in the order of its domains relative to the order present in the preferred CD32B x CD79B bispecific monovalent Fc diabody molecules. However, as in the case of the preferred CD32B x CD79B Fc diabody, the VL Domain of the first polypeptide chain of the alternative CD32B x CD79B bispecific monovalent Fc diabody interacts with the VH Domain of the second polypeptide chain of the alternative CD32B x CD79B bispecific monovalent Fc diabody in order to form a first functional antigen binding site that is specific for the first antigen (i.e., either CD32B or CD79B). Likewise, the VL Domain of the second polypeptide chain of the alternative CD32B x CD79B bispecific monovalent Fc diabody interacts with the VH Domain of the first polypeptide chain of the alternative CD32B x CD79B bispecific monovalent Fc diabody in order to form a second functional antigen binding site that is specific for the second antigen (i.e., either CD79B or CD32B, depending upon the identity of the first antigen). Thus, the selection of the VL and VH Domains of the first and second polypeptide chains are coordinated, such that the two polypeptide chains collectively comprise VL and VH Domains capable of binding to CD32B and CD79B (i.e., they comprise VL_{CD32B}/VH_{CD32B} and VL_{CD79B}/VH_{CD79B}) (FIG. 3C). Collectively, each such VL and VH Domain, and the intervening Linker that separates them, are referred to as an Antigen-Binding Domain of the molecule.

The first polypeptide chain of such alternative CD32B x CD79B Fc diabody comprises, in the N-terminal to C-terminal direction, an amino terminus, the VL Domain of a monoclonal antibody capable of binding to either CD32B or CD79B (i.e., either VL_{CD32B} or VL_{CD79B}), an intervening spacer peptide (Linker 1), a VH Domain of a monoclonal antibody capable of binding to either CD79B (if such first polypeptide chain contains VL_{CD32B}) or CD32B (if such first polypeptide chain contains VL_{CD79B}), a cysteine-containing third intervening spacer peptide (Linker 2), a Heterodimer-Promoting Domain, an optional fourth spacer peptide (Linker 4) to provide improved stabilization to the Heterodimer-Promoting Domain (preferably an E-coil Domain), a cysteine-containing peptide (Peptide 1), an IgG Fc Domain (preferably, the CH2 and CH3 Domains of a knob-containing IgG Fc Region, and a C-terminus. Preferably, the Fc Domain of the first polypeptide chain will have substantially reduced or no ability to bind to FcγRIA (CD64), FcγRIIA (CD32A), FcγRIM (CD32B), FcγRIIIA (CD16a) or FcγRIIIB (CD16b) (relative to the binding exhibited by a wild-type Fc Region) (FIG. 3C).

The second polypeptide chain of such alternative CD32B x CD79B Fc diabody comprises, in the N-terminal to C-terminal direction, an amino terminus, a VL Domain of a monoclonal antibody capable of binding to either CD79B or CD32B (i.e., either VL_{CD79B} or VL_{CD32B}, depending upon the VL Domain selected for the first polypeptide chain of the diabody), an intervening linker peptide (Linker 1), a VH Domain of a monoclonal antibody capable of binding to either CD32B (if such second polypeptide chain contains VL_{CD79B}) or CD32B (if such second polypeptide chain contains VL_{CD32B}), a cysteine-containing spacer peptide (Linker 2), a Heterodimer-Promoting Domain (preferably a K-coil Domain), and a C-terminus (FIG. 3C).

The third polypeptide chain of the preferred CD32B x CD79B Fc diabody comprises, in the N-terminal to C-terminal direction, an amino terminus, a cysteine-containing peptide (Peptide 1), an IgG Fc Domain (preferably, the CH2 and CH3 Domains of a hole-containing IgG Fc Region) having the same isotype as that of the Fc Domain of the first polypeptide chain and a C-terminus. Preferably, the Fc Domain of the third polypeptide chain will have substantially reduced or no ability to bind to FcγRIA (CD64), FcγRIIA (CD32A), FcγRIM (CD32B), FcγRIIIA (CD16a) or FcγRIIIB (CD16b) (relative to the binding exhibited by a wild-type Fc Region) (FIG. 3C).

FIG. 3D shows a variant of such Fc diabody, in which the cysteine-containing Linker 2 (e.g., GGCGGG (SEQ ID NO:16)) has been replaced with a non-cysteine-containing Linker (e.g., AS TKG (SEQ ID NO:15)) and in which the respective Heterodimer-Promoting Domains contain cysteine residues (e.g., EVAACEK-EVAALEK-EVAALEK-EVAALEK (SEQ ID NO:23) and KVAACKE-KVAALKE-KVAALKE-KVAALKE (SEQ ID NO:24)).

### Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of the CD32B x CD79B binding molecules of the present invention, and in particular any of the CD32B x CD79B diabodies or Fc diabodies of the invention or a combination of such agents and a pharmaceutically acceptable carrier. Preferably, compositions of the invention comprise a prophylactically or therapeutically effective amount of one or more molecules of the invention and a pharmaceutically acceptable carrier.

The invention also encompasses pharmaceutical compositions comprising such CD32B x CD79B Binding Molecules, and in particular any of the CD32B x CD79B diabodies or Fc diabodies of the invention and a second therapeutic antibody (e.g., autoimmune or inflammatory disease antigen specific monoclonal antibody) that is specific for a particular autoimmune or inflammatory disease antigen, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The invention also provides a pharmaceutical pack or kit comprising one or more containers filled with CD32B x CD79B Binding Molecules, and in particular any of the CD32B x CD79B diabodies or Fc diabodies of the invention alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises one or more molecules of the invention. In another embodiment, a kit further comprises one or more other prophylactic or therapeutic agents useful for the treatment of an autoimmune or inflammatory disease, in one or more containers. In another embodiment, a kit further comprises one or more antibodies that bind one or more autoimmune or inflammatory disease antigens associated with autoimmune or inflammatory disease. In certain embodiments, the other prophylactic or therapeutic agent is a chemotherapeutic. In other embodiments, the prophylactic or therapeutic agent is a biological or hormonal therapeutic.

### Uses of the Compositions of the Invention

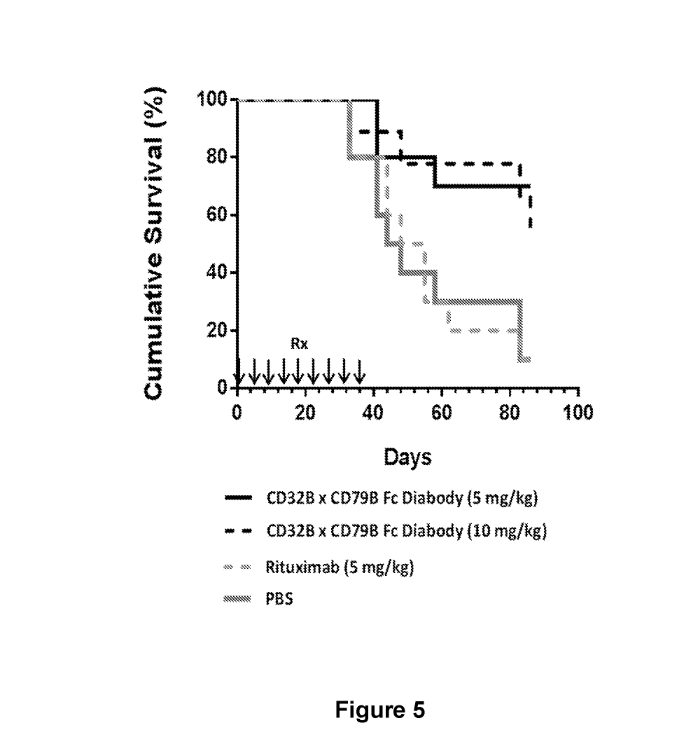

The CD32B x CD79B Binding Molecules, and in particular any of the CD32B x CD79B diabodies or Fc diabodies of the invention have the ability to treat any disease or condition associated with or characterized by the expression of CD79B or having a B-cell component to the disease. Thus, without limitation, pharmaceutical compositions comprising such molecules may be employed in the diagnosis or treatment of autoimmune or inflammatory diseases or conditions. Thus, the invention may be used to treat, prevent, slow the progression of, and/or ameliorate a symptom of B-cell mediated diseases or disorders, including graft rejection, graft-versus-host disease (GvHD), rheumatoid arthritis (RA), multiple sclerosis (MS), and systemic lupus erythematosis (SLE). FIG. 5 shows the ability of the preferred CD32B x CD79B Fc diabody to decrease xenogeneic GvHD in the mouse (see, WO 2015/021089, incorporated herein by reference). Similarly, the CD32B x CD79B Binding Molecules of the invention may be employed to reduce or inhibit B-cell mediated immune responses (e.g., response to antigens, including auto-antigens), to attenuate B-cell activation, and/or to reduce or inhibit B-cell proliferation.

### Methods of Administration

The compositions of the present invention may be provided for the treatment, prophylaxis, and amelioration of one or more symptoms associated with a disease, disorder or infection by administering to a subject an effective amount of a pharmaceutical composition of the invention. In a preferred aspect, such compositions are substantially purified (i.e., substantially free from substances that limit its effect or produce undesired side-effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

The dose of administration and the “dosage regimen” (administration frequency) of the CD32B x CD79B Binding Molecules of the invention may be reduced or altered by enhancing uptake and tissue penetration of such Binding Molecules by modifications such as, for example, lipidation. In one embodiment, a single dosage level (see below) will be administered once or multiple times over a course of therapy. In a second embodiment, the dosage provided over a course of treatment will vary, for example an escalating dosage regimen or a de-escalating dosage regimen. The administered dosage may additionally be adjusted to reflect subject tolerance for the therapy and the degree of therapeutic success associated with the therapy.

The dose of the CD32B x CD79B Binding Molecules of the invention that will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems. The CD32B x CD79B Binding Molecules of the invention are, however, preferably administered at a dosage that is typically at least about 0.1 mg/kg, at least about 0.2 mg/kg, at least about 0.3 mg/kg, at least about 0.5 mg/kg, at least about 1.0 mg/kg, at least about 3.0 mg/kg, at least about 5.0 mg/kg, at least about 7.5 mg/kg, at least about 10.0 mg/kg, at least about 15 mg/kg, at least about 20 mg/kg or more of the subject's body weight. In particular, the CD32B x CD79B Binding Molecules of the invention are administered at a dosage that is about 1.0 mg/kg, about 3.0 mg/kg, about 10.0 mg/kg, about 20.0 mg/kg, or about 30.0 mg/kg. The CD32B x CD79B Binding Molecules, and in particular any of the CD32B x CD79B diabodies or Fc diabodies of the invention are preferably packaged in a hermetically sealed container such as an ampoule or sachette indicating the quantity of such molecules. As used herein, a dosage is said to be “about” a recited dosage if it is described within the significant figures used to describe the recited dosage (for example, a dosage is about 0.1 mg/kg if it is ±0.05 mg/kg of such dosage, and a dosage is about 15 mg/kg if it is ±0.5 mg/kg of such dosage).

The frequency of administration of the CD32B x CD79B Binding Molecules of the invention may range substantially, depending, for example, on patient response or the method of administration. Thus, the compositions of the invention may be administered once a day, twice a day, or three times a day, once a week, twice a week, once every two weeks, once every three weeks, once every four weeks, once a month, once every six weeks, once every twelve weeks, once every two months, once every three months twice a year, once per year, etc. It will also be appreciated that the effective dosage of the molecules used for treatment may increase or decrease over the course of a particular treatment.

However, it is preferred to administer the CD32B x CD79B Binding Molecules of the invention in a course of therapy of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or more than 15 weeks, and such single course of treatment may be repeated 1, 2, 3, 4, 5, or more times. In a preferred embodiment, a subject is treated with a course of therapy of molecules of the invention one time per 2 weeks (Q2W), one time per 3 weeks (Q3W), one time per 4 weeks (Q4W), one time per 5 weeks (Q5W), one time per 6 weeks (Q6W), one time per 7 weeks (Q7W), one time per 8 weeks (Q8W), one time per 9 weeks (Q9W), one time per 10 weeks (Q10W), one time per 11 weeks (Q11W), or one time per 12 weeks (Q2W). It is particularly preferred to administer the CD32B x CD79B Binding Molecules of the invention in a course of therapy one time per 2 weeks (Q2W), one time per 3 weeks (Q3W), or one time per 4 weeks (Q4W). At the conclusion of any such single course of therapy, the therapy may be reinstituted at the same dosage schedule or at a different dosage schedule and may involve the same dosage, or a different dosage, of the administered CD32B x CD79B Binding Molecule. Treatment of a subject with a therapeutically or prophylactically effective amount of the CD32B x CD79B Binding Molecule of the invention thus can comprise a single course of treatment or can include multiple courses of treatment, which may be the same or different from any prior course of treatment.

In preferred embodiments, a CD32B x CD79B Binding Molecule, the CD32B x CD79B Binding Molecules, and in particular any of the CD32B x CD79B diabodies or Fc diabodies of the invention are administered at a dosage of about 3.0 mg/kg, about 10.0 mg/kg, about 20.0 mg/kg, or about 30.0 mg/kg, in a course of therapy Q2W, Q3W, or Q4W.

In one embodiment, the CD32B x CD79B Fc diabodies of the invention are supplied as a dry sterilized lyophilized powder or water free concentrate in a hermetically sealed container and can be reconstituted, e.g., with water or saline to the appropriate concentration for administration to a subject. Preferably, the CD32B x CD79B Binding Molecules, and in particular any of the CD32B x CD79B diabodies or Fc diabodies of the invention are supplied as a dry sterile lyophilized powder in a hermetically sealed container at a unit dosage of at least 1 mg, more preferably at least 2 mg, at least 3 mg, at least 5 mg, at least 10 mg, at least 20 mg, at least 30 mg, at least 50 mg, at least 100 mg, at least 200 mg, at least 300 mg, at least 500 mg, or at least 1000 mg, such that, for example, upon addition of an appropriate volume of carrier an administrable dosage of 0.1 mg/kg, 0.3 mg/kg, 1 mg/kg or 10 mg/kg may be prepared.

The lyophilized CD32B x CD79B Binding Molecules, and in particular any of the CD32B x CD79B diabodies or Fc diabodies of the invention should be stored at between 2° C. and 8° C. in their original container and the molecules should be administered within 12 hours, preferably within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. Preferably, the liquid form of the CD32B x CD79B Binding Molecules of the invention is supplied in a hermetically sealed container in which the molecules are present at a concentration of least 1 μg/ml, more preferably at least 2.5 mg/ml, at least 5 mg/ml, at least 10 mg/ml, at least 50 mg/ml, at least 100 mg/ml, or at least 200 mg/ml.

In one embodiment, the dosage of the CD32B x CD79B Binding Molecules of the invention administered to a patient may be calculated for use as a single agent therapy. In another embodiment, the Binding Molecules of the invention are used in combination with other therapeutic compositions and the dosage administered to a patient are lower than when such Binding Molecules are used as a single agent therapy.

Preferred methods of administering the CD32B x CD79B Binding Molecules, and in particular any of the CD32B x CD79B diabodies or Fc diabodies of the invention include, but are not limited to, parenteral administration (e.g., intradermal, intramuscular, intraperitoneal, intravenous and subcutaneous), epidural, and mucosal (e.g., intranasal and oral routes). In a specific embodiment, the molecules of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a molecule of the invention, care must be taken to use materials to which the molecule does not absorb.

### Embodiments of the Invention

Provided hereafter are non-limiting examples of certain Embodiments of the invention.

### Embodiment 1

A method of treating an inflammatory disease or condition that comprises administering a therapeutically effective amount of a CD32B x CD79B Binding Molecule to a subject in need thereof, wherein said CD32B x CD79B Binding Molecule is capable of immunospecifically binding an epitope of CD32B and an epitope of CD79B, and wherein said CD32B x CD79B Binding Molecule is administered at a dose of between about 1 mg/kg and about 30 mg/kg, and at a dosage regimen of between one dose per week and one dose per 8 weeks.

### Embodiment 2

A method of reducing or inhibiting B-cell mediated immune responses that comprises administering a therapeutically effective amount of a CD32B x CD79B Binding Molecule to a subject in need thereof, wherein said CD32B x CD79B Binding Molecule is capable of immunospecifically binding an epitope of CD32B and an epitope of CD79B, and wherein said CD32B x CD79B Binding Molecule is administered at a dose of between about 1 mg/kg and about 30 mg/kg, and at a dosage regimen of between one dose per week and one dose per 8 weeks.

### Embodiment 3

A method of attenuating B-cell activation that comprises administering a therapeutically effective amount of a CD32B x CD79B Binding Molecule to a subject in need thereof, wherein said CD32B x CD79B Binding Molecule is capable of immunospecifically binding an epitope of CD32B and an epitope of CD79B, and wherein said CD32B x CD79B Binding Molecule is administered at a dose of between about 1 mg/kg and about 30 mg/kg, and at a dosage regimen of between one dose per week and one dose per 8 weeks.

### Embodiment 4

A method of reducing or inhibiting B-cell proliferation that comprises administering a therapeutically effective amount of a CD32B x CD79B Binding Molecule to a subject in need thereof, wherein said CD32B x CD79B Binding Molecule is capable of immunospecifically binding an epitope of CD32B and an epitope of CD79B, and wherein said CD32B x CD79B Binding Molecule is administered at a dose of between about 1 mg/kg and about 30 mg/kg, and at a dosage regimen of between one dose per week and one dose per 8 weeks.

### Embodiment 5

The method of any one of Embodiments 1-4, wherein said CD32B x CD79B Binding Molecule is administered at a dose of about 1 mg/kg.

### Embodiment 6

The method of any one of Embodiments 1-4, wherein said CD32B x CD79B Binding Molecule is administered at a dose of about 3 mg/kg.

### Embodiment 7

The method of any one of Embodiments 1 or 4, wherein said CD32B x CD79B Binding Molecule is administered at a dose of about 10 mg/kg.

### Embodiment 8

The method of any one of Embodiments 1-7, wherein said dosage regimen is one dose per 2 weeks (Q2W).

### Embodiment 9

The method of any one of Embodiments 1-7, wherein said dosage regimen is one dose per 3 weeks (Q3W).

### Embodiment 10

The method of any one of Embodiments 1-7, wherein said dosage regimen is one dose per 4 weeks (Q4W).

### Embodiment 11

The method of any one of Embodiments 1-10, wherein said CD32B x CD79B Binding Molecule is a bispecific antibody that binds an epitope of CD32B and an epitope of CD79B, or a molecule that comprises the CD32B- and CD79B-binding domains of said antibody.

### Embodiment 12

The method of any one of Embodiments 1-11, wherein said CD32B x CD79B Binding Molecule is a CD32B x CD79B bispecific diabody that binds an epitope of CD32B and an epitope of CD79B.

### Embodiment 13

The method of Embodiment 12, wherein said CD32B x CD79B bispecific diabody is a CD32B x CD79B Fc diabody.

### Embodiment 14

The method of any one of Embodiments 1, or 5-13, wherein said inflammatory disease or condition is an autoimmune disease.

### Embodiment 15

The method of Embodiment 14, wherein said autoimmune disease is selected from the group consisting of: Addison's disease, autoimmune hepatitis, autoimmune inner ear disease myasthenia gravis, Crohn's disease, dermatomyositis, familial adenomatous polyposis, graft vs. host disease (GvHD), Graves' disease, Hashimoto's thyroiditis, lupus erythematosus, multiple sclerosis (MS); pernicious anemia, Reiter's syndrome, rheumatoid arthritis (RA), Sjogren's syndrome, systemic lupus erythematosus (SLE), type 1 diabetes, primary vasculitis (e.g., polymyalgia rheumatic, giant cell arteritis, Behcets), pemphigus, neuromyelitis optica, anti-NMDA receptor encephalitis, Guillain-Barré syndrome, chronic inflammatory demyelinating polyneuropathy (CIDP), Grave's opthalmopthy, IgG4 related diseases, idiopathic thrombocytopenic purpura (ITP), and ulcerative colitis.

### Embodiment 16

The method of Embodiment 15, wherein said inflammatory disease or condition is GvHD, RA, MS, or SLE.

### Embodiment 17

The method of any one of Embodiments 1-16, wherein the serum level of an immunoglobulin is reduced by day 36 after administration of a first dose of said CD32B x CD79B Binding Molecule.

### Embodiment 18

The method of Embodiment 17, wherein said immunoglobulin is IgM, IgA or IgG.

### Embodiment 19

The method of Embodiment 18, wherein said immunoglobulin is IgM.

### Embodiment 20

The method of any one of Embodiments 1-19, wherein BCR-mediated peripheral B-cell activation is inhibited by 24 hours after administration of a first dose of said CD32B x CD79B Binding Molecule, wherein said B-cell activation is determined by an ex vivo calcium mobilization assay.

### Embodiment 21

The method of Embodiment 20, wherein said BCR-mediated B-cell activation is inhibited by at least 50%, and wherein said inhibition is sustained for at least 6 days.

### Embodiment 22

The method of any one of Embodiments 1-21, wherein at least 20% of CD32B x CD79B binding sites on peripheral B-cell are occupied 6 hours after administration of a first dose of said CD32B x CD79B Binding Molecule.

### Embodiment 23

The method of any one of Embodiments 1-22, wherein:
(A) the expression of CD40 on B-cells is down regulated; and/or
(B) CD40 mediated IgG secretion is inhibited.

### Embodiment 24

The method of any one of Embodiments 1-23, wherein said subject is a human.

### Embodiment 25

The method of any one of Embodiments 1-24, wherein said CD32B x CD79B Binding Molecule comprises:
(A) a VL_{CD32B} Domain that comprises the amino acid sequence of SEQ ID NO:30; and
(B) a VH_{CD32B} Domain that comprises the amino acid sequence of SEQ ID NO:31.

### Embodiment 26

The method of any one of Embodiments 1-24, wherein said CD32B x CD79B Binding Molecule comprises:
(A) a VL_{CD79B} Domain that comprises the amino acid sequence of SEQ ID NO:32; and
(B) a VH_{CD79B} Domain that comprises the amino acid sequence of SEQ ID NO:33.

### Embodiment 27

The method of any one of Embodiments 1-24, wherein said CD32B x CD79B Binding Molecule comprises:
(A) a VL_{CD32B} Domain that comprises the amino acid sequence of SEQ ID NO:30;
(B) a VH_{CD32B} Domain that comprises the amino acid sequence of SEQ ID NO:31;
(C) a VL_{CD79B} Domain that comprises the amino acid sequence of SEQ ID NO:32; and
(D) a VH_{CD79B} Domain that comprises the amino acid sequence of SEQ ID NO:33.

### Embodiment 28

The method of any one of Embodiments 25-27, wherein said CD32B x CD79B Binding Molecule is a bispecific antibody or a bispecific antigen-binding fragment thereof.

### Embodiment 29

The method of any one of Embodiments 25-27, wherein said CD32B x CD79B Binding Molecule is a CD32B x CD79B bispecific diabody.

### Embodiment 30

The method of Embodiment 29, wherein said CD32B x CD79B bispecific diabody is a CD32B x CD79B Fc diabody.

### Embodiment 31

The method of Embodiment 30, wherein said CD32B x CD79B Fc diabody comprises:
(A) a first polypeptide chain that comprises the amino acid sequence of SEQ ID NO:39;
(B) a second polypeptide chain that comprises the amino acid sequence of SEQ ID NO:41; and
(C) a third polypeptide chain that comprises the amino acid sequence of SEQ ID NO:44.

Having now generally described the invention, the same will be more readily understood through reference to the following examples, which are provided by way of illustration and are not intended to be limiting of the present invention unless specified.

### EXAMPLES

### Example 1

### Construction of CD32B x CD79B Bispecific Monovalent Fc Diabodies and Control Diabodies

A CD32B x CD79B Fc diabody was prepared and employed as an exemplary CD32B x CD79B Binding Molecule of the invention. The CD32B x CD79B Fc diabody comprised three polypeptide chains having the amino acid sequences shown in Table 1:

TABLE 1

| | Substituent Polypeptides (in the |
|---|---|
| Preferred CD32B × CD79B | N-Terminal to C-Terminal |
| Bispecific Fc Diabody | Direction) |
| First Polypeptide Chain | SEQ ID NO: 25 |
| (SEQ ID NO: 39) | SEQ ID NO: 38 |
| | SEQ ID NO: 28 |
| | SEQ ID NO: 30 |
| | SEQ ID NO: 14 |
| | SEQ ID NO: 33 |
| | SEQ ID NO: 16 |
| | SEQ ID NO: 21 |
| | SEQ ID NO: 29 |
| Second Polypeptide Chain | SEQ ID NO: 32 |
| (SEQ ID NO: 41) | SEQ ID NO: 14 |
| | SEQ ID NO: 31 |
| | SEQ ID NO: 16 |
| | SEQ ID NO: 22 |
| Third Polypeptide Chain | SEQ ID NO: 25 |
| (SEQ ID NO: 44) | SEQ ID NO: 43 |

The above-described CD32B x CD79B Fc diabody was found to be capable of simultaneously binding to CD32B and to CD79B. Methods for forming bispecific monovalent diabodies are provided in WO 2006/113665, WO 2008/157379, WO 2010/080538, WO 2012/018687, WO 2012/162068 and WO 2012/162067.

### Example 2

### Evaluation of the In Vivo Administration of CD32B x CD79B Bispecific Fc Diabodies

In order to assess the safety and tolerability of the CD32B x CD79B Binding Molecules of the present invention, the CD32B x CD79B Fc diabody of Example 1 was administered to healthy human subjects, age 18-50, having a BMI of 18-30 kg/m². The subjects did not include pregnant women or women of child-bearing potential. Additionally, the subjects did not include individuals having significant acute or chronic medical illness, individuals who had used any prescription drugs within 4 weeks of dosing or who had used over-the-counter drugs within 1 week of dosing, individuals who smoke more than 10 cigarettes per day, individuals having tuberculosis, hepatitis B infections, hepatitis C infections or HIV infections, individuals having a known history of autoimmune or vascular disorders, or individuals having a positive drug test result. The subjects additionally did not include individuals having a corrected QT (QTc) greater than 450 msec, a heart rate less than 45 bpm or greater than 120 bpm, a systolic blood pressure (SBP) greater than 140 mm Hg, or a diastolic blood pressure (DBP) greater than 90 mm Hg. The baseline demographics of the subjects involved in the study are presented in Table 2.

TABLE 2

| Attribute | Value |
|---|---|
| Number | 49 |
| Mean Age (years) | 33.4 ± 7.4 |
| Gender (M:F) | 48:1 |
| Race | |
| White | 12 |
| Black | 35 |
| Other | 2 |
| Mean Weight (kg) | 78.0 ± 12.1 |
| Mean Height (cm) | 177.5 ± 7.8 |

The evaluation comprised the use of 6 dosage cohorts (each composed of 8 subjects, of which Subjects 1-6 would be administered the CD32B x CD79B Fc diabody and Subjects 7-8 would be treated with placebo). Within each cohort, the administration of the CD32B x CD79B Fc diabody was staggered, such that Subject 2 received treatment 24 hours after Subject 1, Subjects 3-5 received treatment 24 hours after Subject 2, and Subjects 7-8 received treatment 24 hours after Subjects 3-5. The dosage cohorts are described in Table 3.

TABLE 3

| Dose Cohort | CD32B × CD79B Fc Diabody Dosage |
|---|---|
| 1 | 0.01 mg/kg |
| 2 | 0.1 mg/kg |
| 3 | 0.3 mg/kg |
| 4 | 1.0 mg/kg |
| 5 | 3.0 mg/kg |
| 6 | 10.0 mg/kg |

Administered subjects were monitored for CD32B x CD79B Fc diabody-associated adverse effects: second or third degree heart block, ventricular arrhythmia (including Torsade de Pointes) or ≥Grade 3 adverse event according to the Guidance of Toxicity Grading Scale for healthy adult and adolescent volunteers enrolled in preventive vaccine clinical trials. For issues not covered (e.g., infusion-related reaction) NCI CTCAE v4.03 was used. Fifteen adverse events were noted, of which 4 were deemed to be associated with the administration of the CD32B x CD79B Fc diabody. Table 4 summarizes the observed adverse events.

TABLE 4

| Adverse Event | All Events | | Related | |
|---|---|---|---|---|
| (MedRa Preferred Term) | All | ≥Gr 3 | All | ≥Gr 3 |
| Conjunctival Haemorrhage | 1 | — | — | — |
| Ocular Hyperemia | 1 | — | — | — |
| Pupils Unequal | 1 | — | — | — |
| Nausea | 1 | — | 1 | — |
| Chills | 1 | — | — | — |
| Vessel puncture site bruise | 1 | — | — | — |
| Folliculitis | 1 | — | 1 | — |
| Upper respiratory tract infection | 2 | — | 2 | — |
| Viral upper respiratory tract infection | 2 | — | — | — |
| Contusion | 1 | — | — | — |
| Ligament sprain | 1 | — | — | — |
| Limb injury | 1 | — | — | — |
| Muscle strain | 1 | — | — | — |
| Periorbital contusion | 1 | — | — | — |
| Muscle twitching | 1 | — | — | — |
| Headache | 3 | — | 3 | — |
| Somnolence | 1 | — | 1 | — |
| Terminal insomnia | 1 | — | — | — |
| Nasal congestions | 1 | — | — | — |
| Rhinorrhea | 1 | — | 1 | — |
| Dry skin | 1 | — | — | — |
| Night sweats | 1 | — | 1 | — |
| Pruritus | 1 | — | — | — |
| Rash | 1 | — | 1 | — |
| Hypertension | 1 | 1 | — | — |

### A. Pharmacodynamics Effects of the Fc Diabody on Humoral Immune Responses

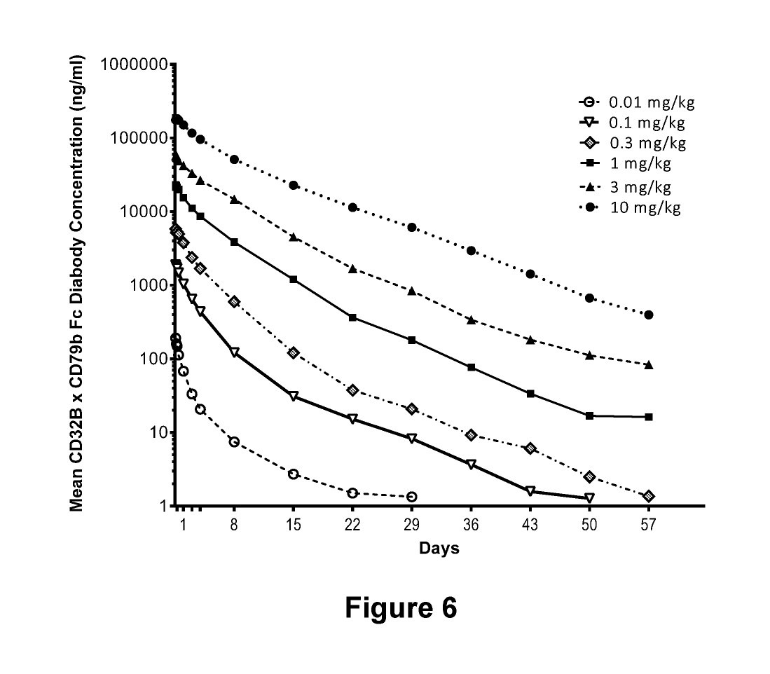

FIG. 6 shows the pharmacokinetics of the exemplary CD32B x CD79B Binding Molecule upon administration to human subjects. Binding Molecule concentrations were measured by a validated ELISA assay and PK parameters were calculated from non-compartmental analyses. As indicated in the Figure, subjects received CD32B x CD79B Fc Diabody at dosages of 0.01 mg/kg, 0.1 mg/kg, 0.3 mg/kg, 1 mg/kg, 3 mg/kg or 10 mg/kg body weight, and the serum diabody concentration was measured over a maximum of 57 days post-administration. The serum concentration of the diabody, the maximum serum concentration (Cₘₐₓ) and the AUC (area under curve) were all found to increase with increasing dose concentration. The half-life of the diabody ranged from 4-8 days. A rapid disposition of diabody was observed at the lowest dose (0.01 mg/kg). The mean clearance time (CL) ranged from 1.426 mL/h/kg (0.01 mg/kg dose) to 0.350 mL/h/kg (10 mg/kg dose), and the relationship between CL and dose was non-linear, with CL decreasing with increasing dose. The steady-state distribution volume (Vss), an indication of the distribution of diabody within the blood volume, was found to be independent of dose. The serum half-life (T_{1/2}) ranged from 92 hrs (˜4 days) for diabody administered at the 0.03 mg/kg dose to 191 hrs (˜8 days) for diabody administered at the 10 mg/kg dose, and increased with increasing dose, consistent with dose-dependent decrease in CL. Disruption in PK profiles were noted, suggesting the presence of anti-drug-antibodies (ADA). The pharmacokinetic data are summarized in Table 5.

TABLE 5

| Dose | Cₘₐₓ | AUC | CL | Vₛₛ | T_{1/2} |
|---|---|---|---|---|---|
| (mg/kg) | (μg/mL) | (h * μg/mL) | (mL/h/kg) | (mL/kg) | (Hr) |
| 0.01 | 0.192 | 7.423 | 1.426 | 154 | 156 |
| 0.1 | 1.928 | 113.861 | 0.917 | 87 | 140 |
| 0.3 | 5.903 | 423.948 | 0.722 | 65 | 92 |
| 1.0 | 23.701 | 2255.786 | 0.461 | 56 | 143 |
| 3.0 | 58.825 | 7919.163 | 0.387 | 63 | 172 |
| 10.0 | 197.633 | 29206.316 | 0.350 | 68 | 191 |

### B. Ability to Bind to CD32B and CD79B on Peripheral B-Cells

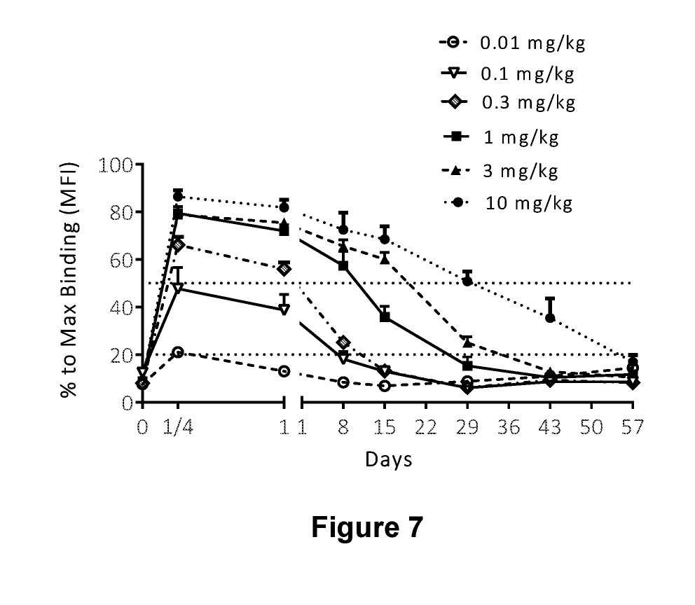

The administered CD32B x CD79B Binding Molecule was found to be capable of binding to peripheral B-cells, showing a maximum occupancy≥1 mg/kg body weight, with a sustained 50% B-cell occupancy at higher dose levels. Sustained 20% B-cell occupancy was observed at ≥0.3 mg/kg. FIG. 7 summarizes the ex vivo flow cytometric analysis of binding to peripheral B-cells over the study course.

### C. Evaluation of the Activation Status of Peripheral B-Cells and B-Cell Subsets

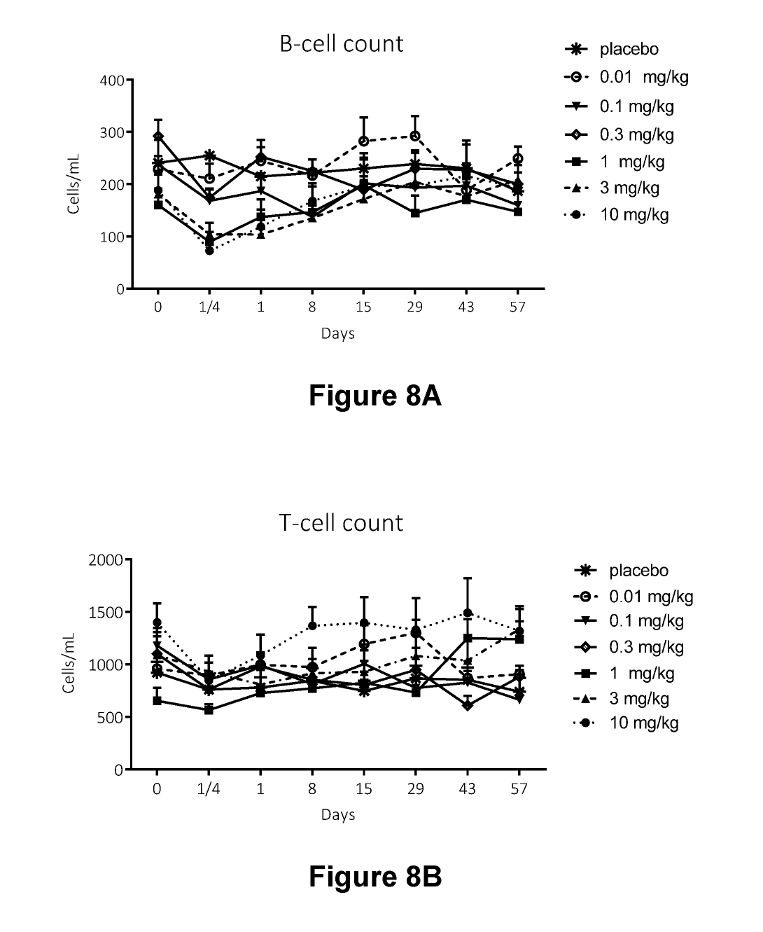

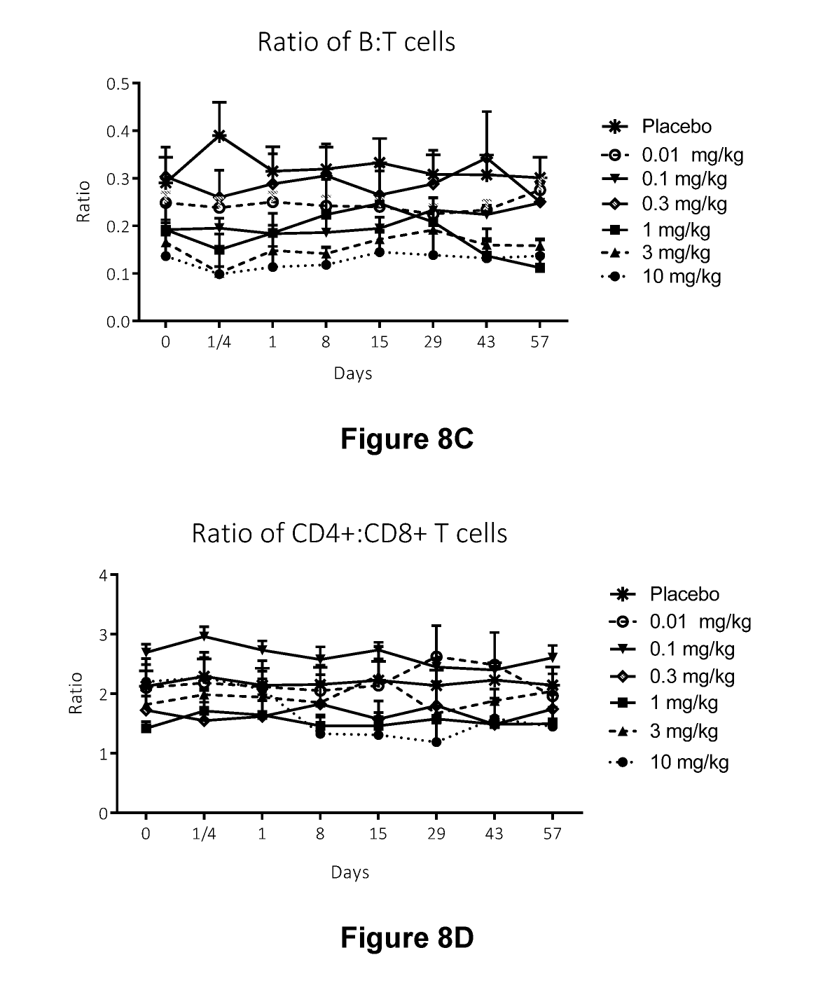

Flow cytometric analysis was conducted in order to assess whether the administration of the diabody was associated with sustained changes in the B-cell count (FIG. 8A), the T-cell count (FIG. 8B), the ratio of B-cells to T cells (FIG. 8C), and the ratio of CD4+ T cells to CD8+ T cells (FIG. 8D). As shown in FIGS. 8A-8B, no sustained changes in peripheral T-cell populations was observed, a transient reduction in B-cell populations was observed at higher doses in this study. As shown in FIGS. 8C-8D, no sustained changes in the ratios of such peripheral B- and T-cell populations was observed.

### D. Response Assessment of Peripheral B-Cells to Ex Vivo BCR Stimulation

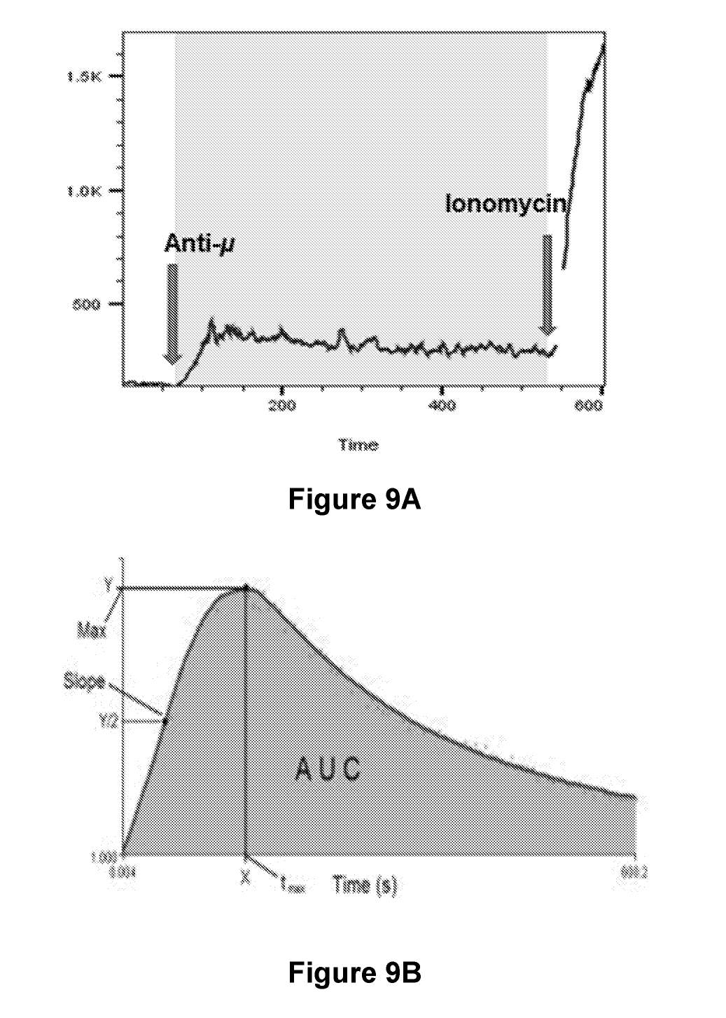

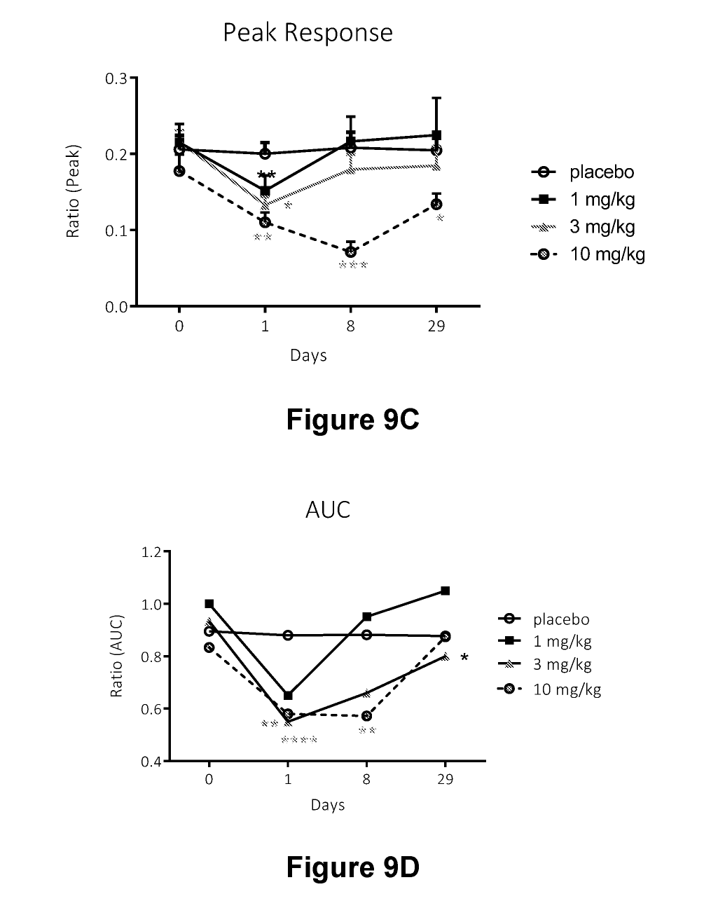

An ex vivo calcium mobilization assay was conducted in order to evaluate the B-cell function of recipients of the exemplary CD32B x CD79B Binding Molecule. In brief, PBMC were freshly isolated from blood samples collected at various pre- and post-dose time points, and a Ca⁺⁺ flux was induced by BCR ligation. Ionomycin was introduced in order to induce a maximum Ca⁺⁺ flux. The values of AUC or Peak were normalized with values generated by ionomcycin, such that the ratio(AUC)=AUC (IgM)/AUC (ionomycin). FIGS. 9A-9B illustrate the experimental procedure and data analysis methods employed in this study.

Treatment with the exemplary CD32B x CD79B Binding Molecule was found to reduce calcium flux in response to BCR ligation by anti-IgM antibody, thus demonstrating the inhibitory activity of the CD32B x CD79B Binding Molecules of the present invention on peripheral B-cells (FIGS. 9C-9D).

### E. CD32B x CD79B Binding Molecules Down-Regulate BCR Expression on CD27⁺ Memory B-Cells

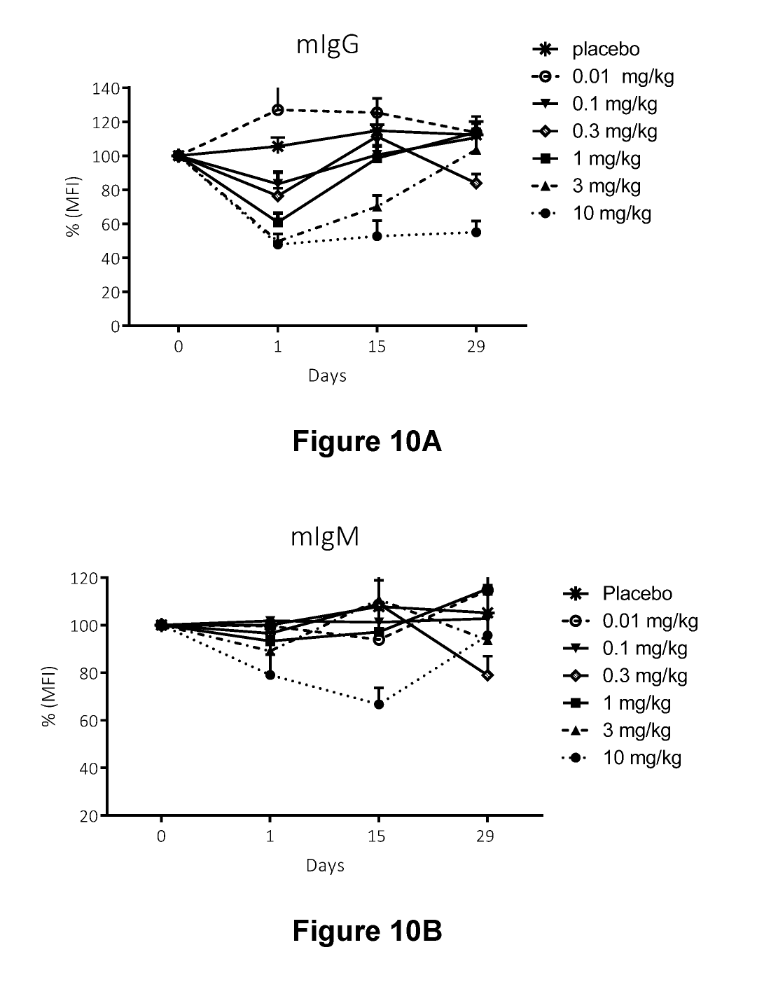

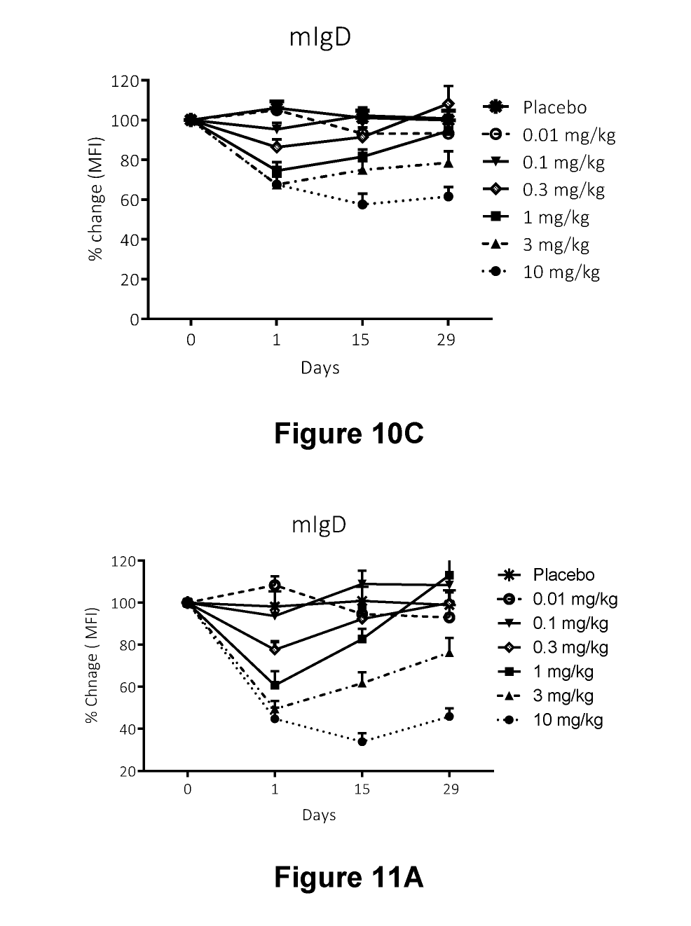

In order to further assess the effect of administration of the exemplary CD32B x CD79B Binding Molecule, flow cytometry was used to determine membrane-bound immunoglobulin levels. FIGS. 10A-10C show that administration of CD32B x CD79B Binding Molecules down-regulates BCR expression on CD27⁺ memory B-cells as determined by the expression of membrane-bound IgG (mIgG) (FIG. 10A), membrane-bound IgM (mIgM) (FIG. 10B), and membrane-bound IgD (mIgD) (FIG. 10C).

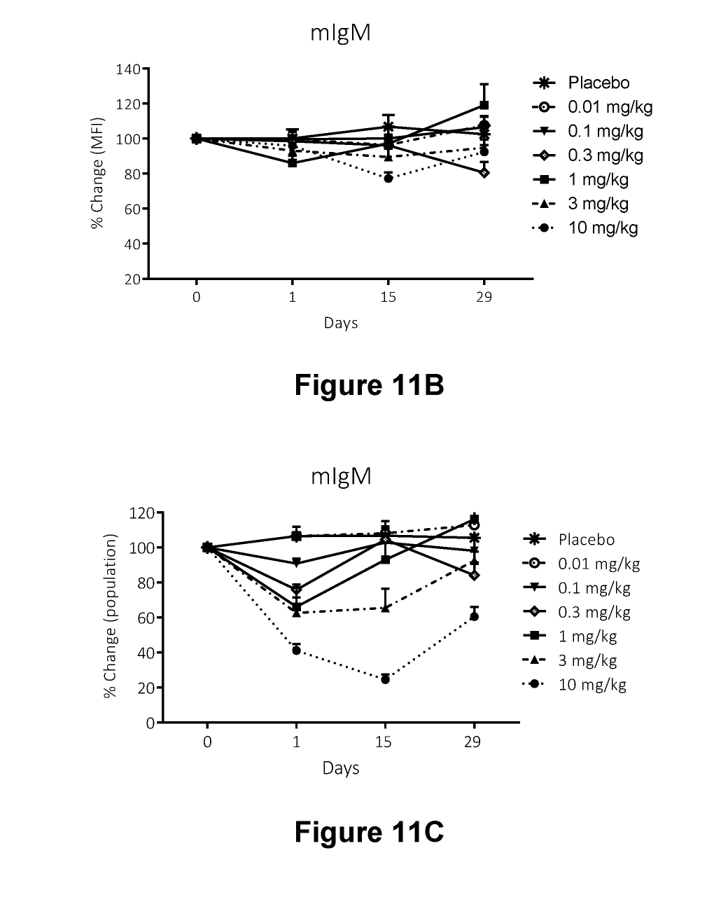

Similar studies established that administration of CD32B x CD79B Binding Molecules down-regulates BCR expression on CD27-naïve memory B-cells (FIG. 11A: membrane-bound IgD (mIgD); FIG. 11B: membrane-bound IgM; FIG. 11C: percent change in membrane-bound IgM (mIgM)).

### F. CD32B x CD79B Binding Molecules Modulate Serum Ig Levels

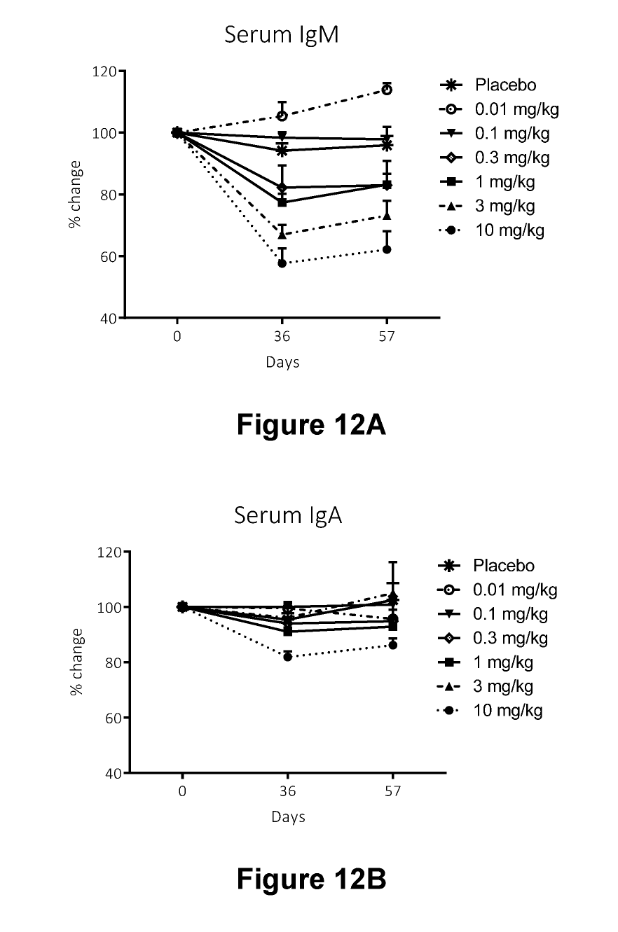

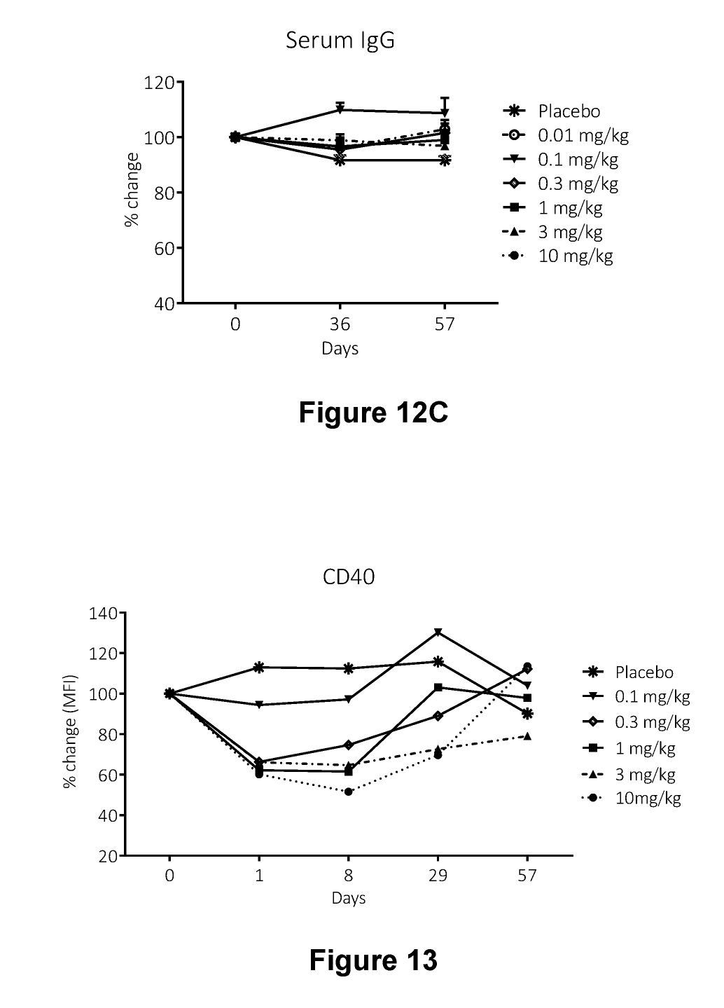

The effect of the administration of the CD32B x CD79B Binding Molecule on the serum levels of IgM, IgA and IgG immunoglobulins was evaluated. The Binding Molecule was found to modulate the serum levels of these immunoglobulins with IgM levels exhibiting the largest reduction and IgG level being largely maintained (FIGS. 12A-12C, respectively). The sparing of IgG is desirable. The decreased serum IgM levels suggests an impact on plasmablasts. These results are consistent with expression of CD79B which is not expressed on plasma cells.

### G. CD32B x CD79B Binding Molecules Reduce the Levels of the Co-Stimulatory Molecule, CD40

Administration of the CD32B x CD79B Binding Molecule was found to reduce CD40 surface expression levels on B-cells (FIG. 13A) as determined through a flow cytometric analysis of the surface co-stimulation molecules of peripheral B-cells.

A single intravenous dose of an exemplary Binding Molecule (the CD32B x CD79B Fc Diabody of Example 1) did not decrease peripheral B-cell count substantially, indicating that the Binding Molecules of the present invention do not deplete B-cells. Flow cytological analyses demonstrated that the Binding Molecules of the present invention are capable of binding to peripheral B-cells. Full saturation of such binding sites on peripheral B-cells was observed at ≥1 mg/kg does levels. The duration of such binding was found to be associated with the employed dose level.

The Binding Molecules of the present invention mediated multiple pharmacodynamics dose-dependent effects, including:
1. reduction in ex vivo BCR-induced Ca⁺⁺ mobilization;
2. down-regulation of surface IgG-BCR expression on CD27⁺ memory B-cell subset;
3. decrease of IgM⁺ naïve B-cell population;
4. down-regulation of surface IgD-BCR expression on naïve B-cells; and
5. down-regulation of CD40 expression on B-cells.

The Binding Molecules of the present invention mediated multiple favorable pharmacodynamics characteristics, suitable for clinical dosing. Peripheral B-cells are fully saturated with the exemplary CD32B x CD79B Fc Diabody of Example 1 at ≥1 mg/kg dose levels, and the duration of binding correlated with increasing dosage level with sustained 20% occupancy observed at ≥0.3 mg/kg dose levels. The Binding Molecules of the present invention did not deplete peripheral B-cells, and were found to down-modulate B-cell activity in a dose-dependent manner:
1. reducing BCR-mediated Ca2+ influx;
2. down-regulating surface immunoglobulin expression;
3. reducing the serum IgM level; and
4. down-regulating CD40 expression.

The data supports the utility of the present invention in treating inflammatory diseases, conditions and disorders, and in particular, in treating autoimmune disease.

### Example 3

### Investigation of Pharamcokinetic and Pharmacodynamic Properties of CD32B X CD79B Binding Molecules

An Eₘₐₓ (maximal effect) PK/PD model was employed in order to more fully investigate the pharamcokinetic (PK) and pharmacodynamic (PD) properties of the CD32B x CD79B Binding Molecules of the invention. In brief, B-cells were incubated in the presence of various concentrations of the Binding Molecule, and the concentrations of Binding Molecule resulting in 50%, 60%, 80% and 90% inhibition of Eₘₐₓ were determined.

Recipient subjects exhibited baseline values that ranged from 6.3% to 15.6% (Mean=9.4%). The concentration producing 50% of maximal response (EC50) was 1677 ng/mL. As the concentration of administered Binding Molecule increased, the percent B-cell binding reached a plateau exhibiting a maximum response (Emax) for the % B-cell binding at 73.1%. The data is shown in Table 6. Based on the in vivo humanized mouse model, the predicted target concentration demonstrating complete inhibition of IgG and IgM is 1,500 ng/mL. The PK/PD relationship in the study suggests that a concentration≥EC50 could be a potential target concentration for therapeutic use.

TABLE 6

| % Eₘₐₓ | Concentration (ng/ml) |
|---|---|
| 50 | 1677 |
| 60 | 2658 |
| 70 | 4392 |
| 80 | 8103 |
| 90 | 20364 |

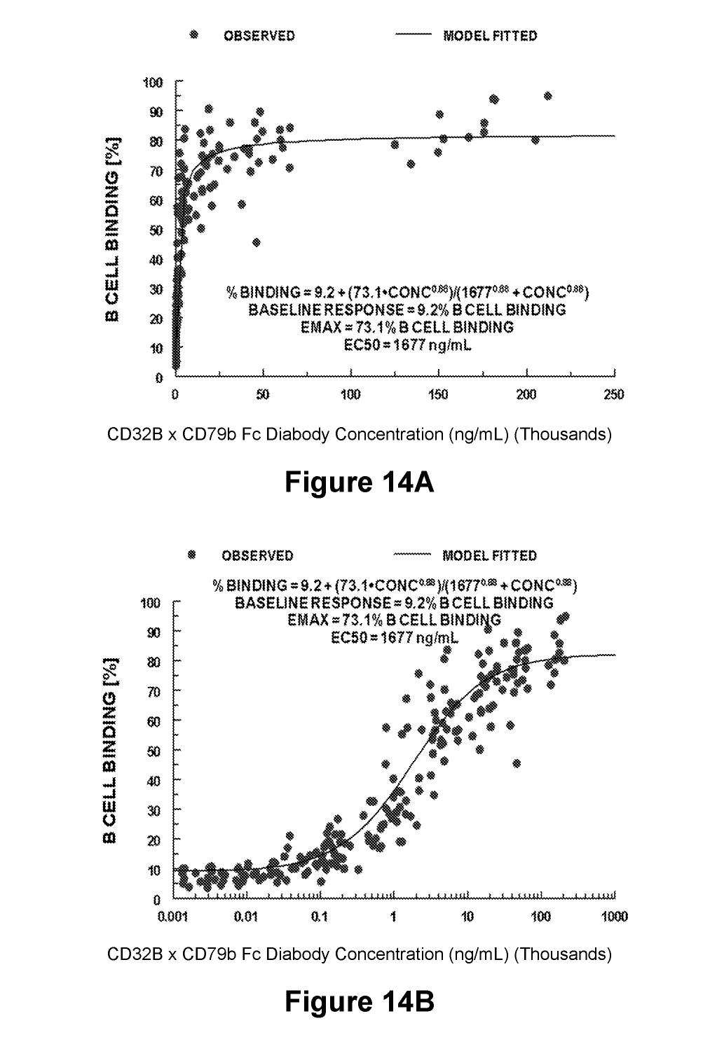

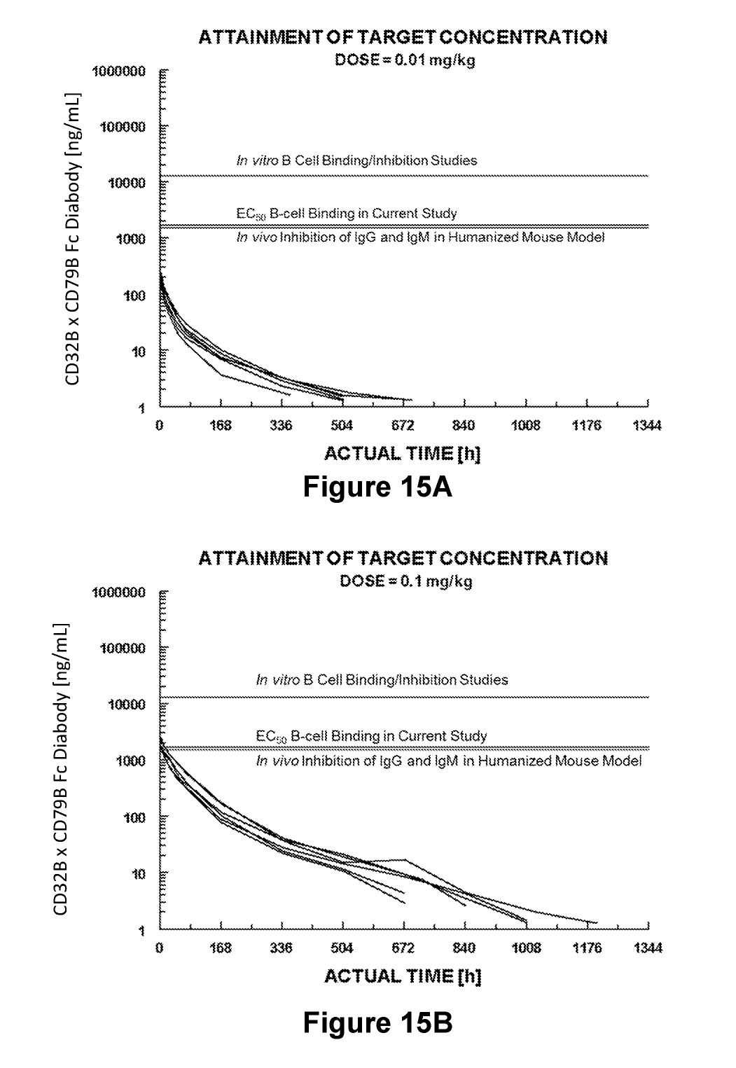

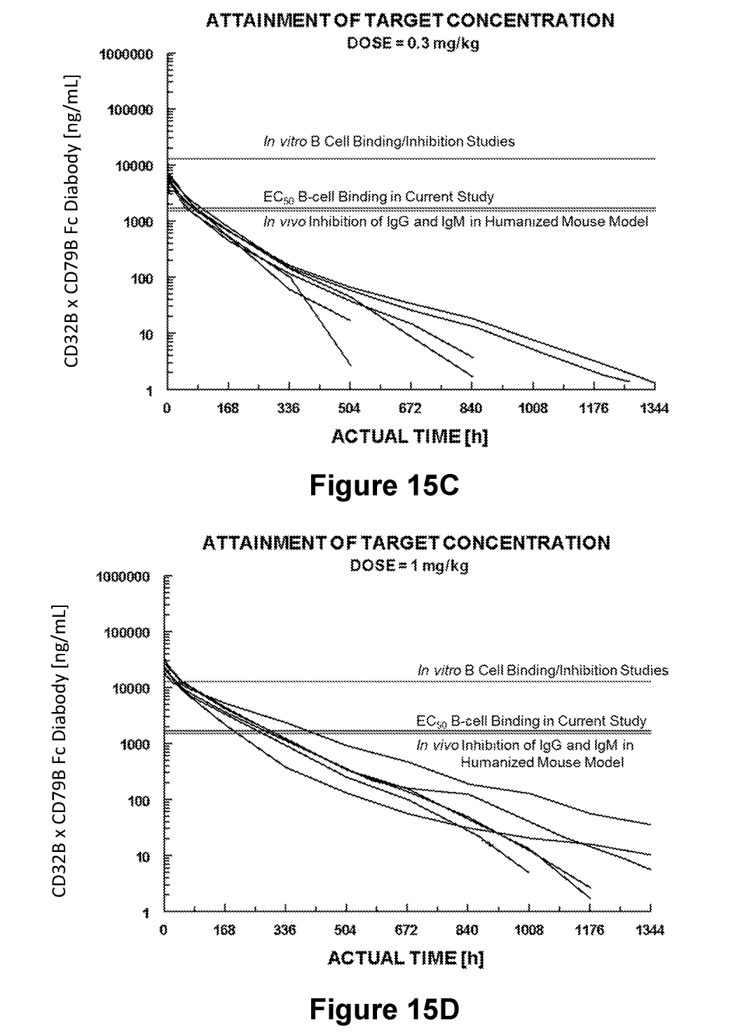

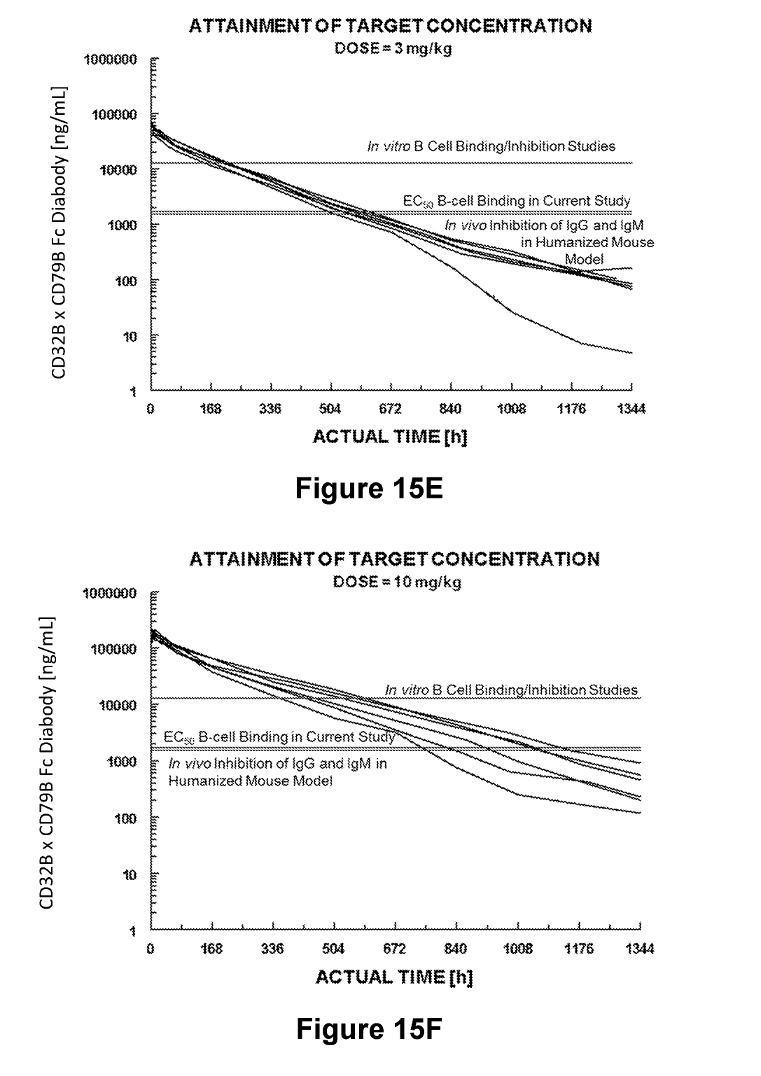

FIGS. 14A-14B show the data (at two different concentration ranges). FIGS. 15A-15F depict preclinical target concentrations with superimposed CD32B x CD79B Binding Molecule pharmacokinetic profiles in humans to identify the doses that would attain the target concentrations (attainment of target concentration at dosage of 0.01 mg/kg (FIG. 15A); at dosage of 0.1 mg/kg (FIG. 15B); at dosage of 0.3 mg/kg (FIG. 15C); at dosage of 1 mg/kg (FIG. 15D); at dosage of 3 mg/kg (FIG. 15E); at dosage of 10 mg/kg (FIG. 15F)).

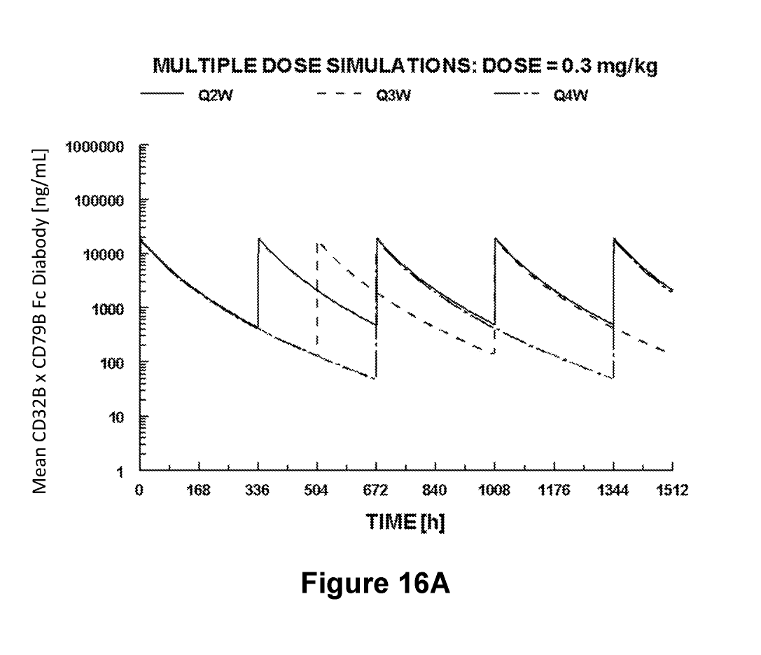

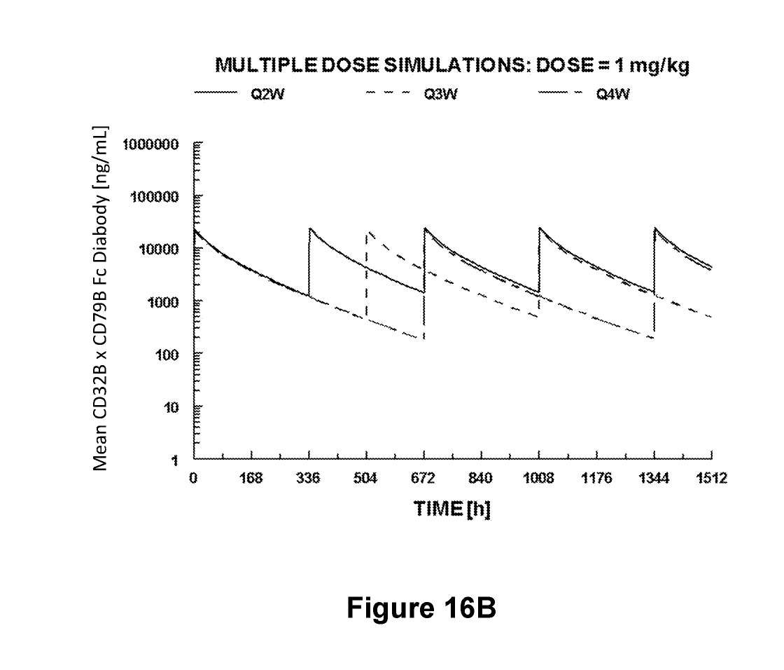

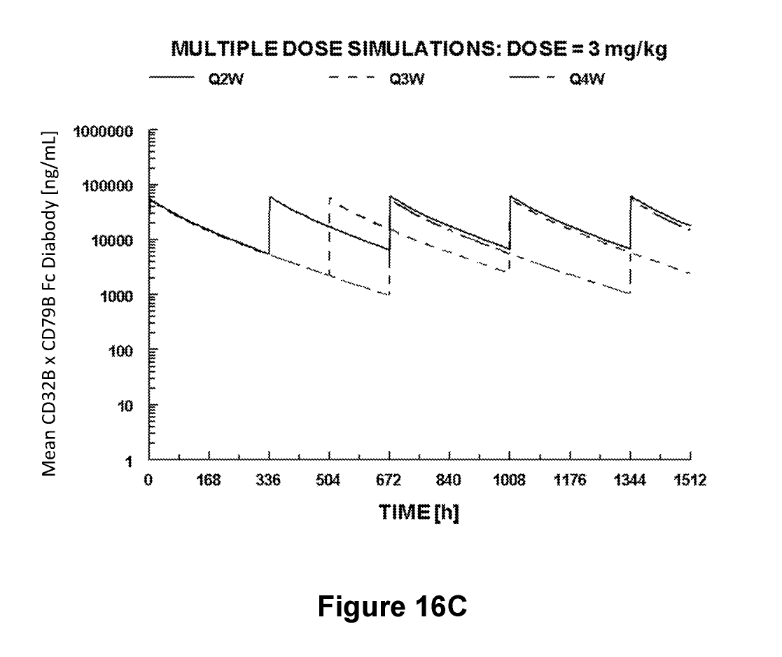

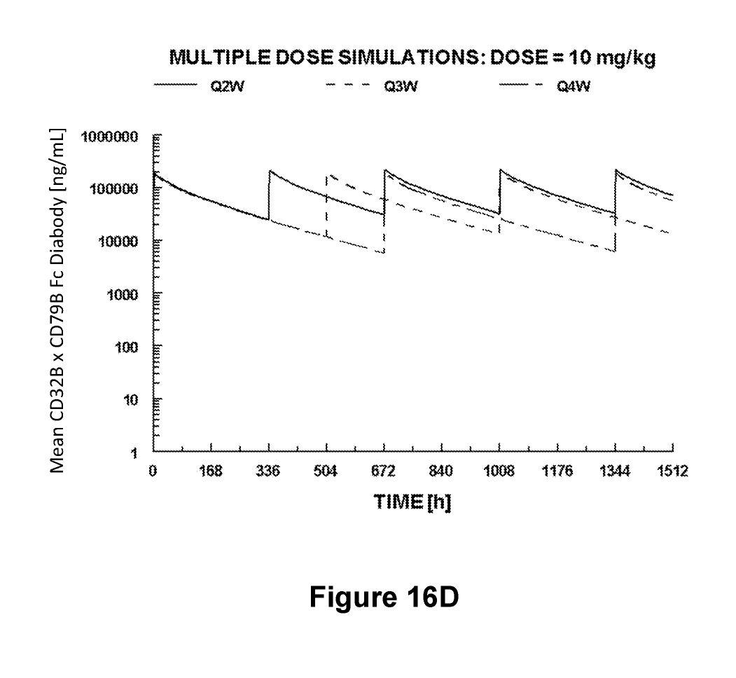

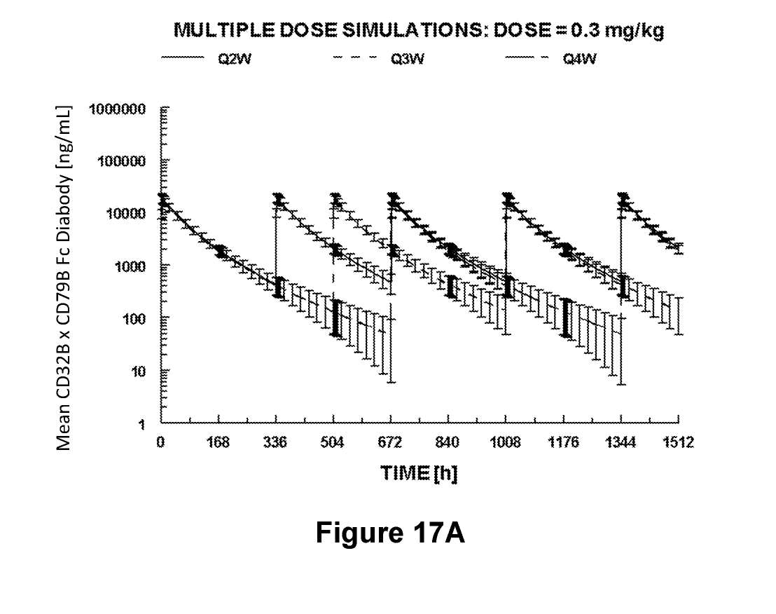

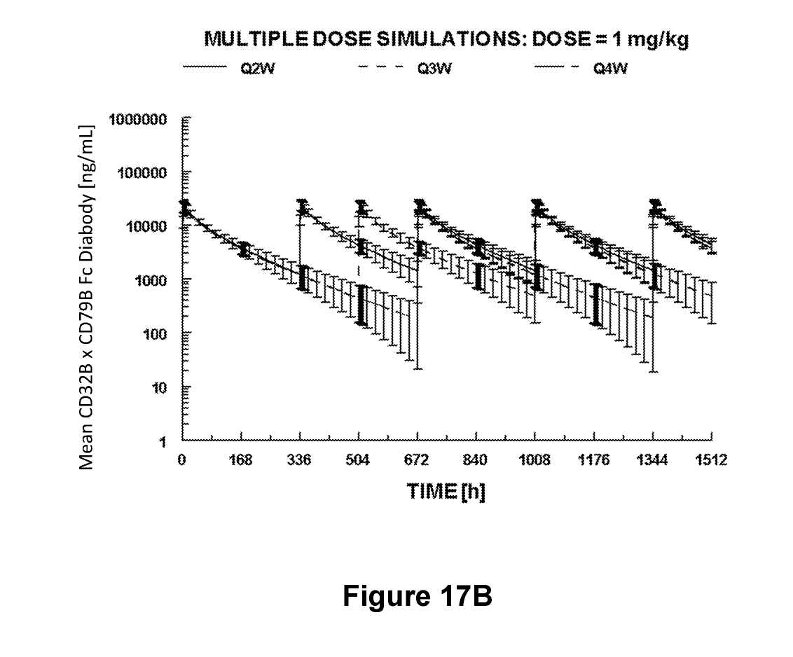

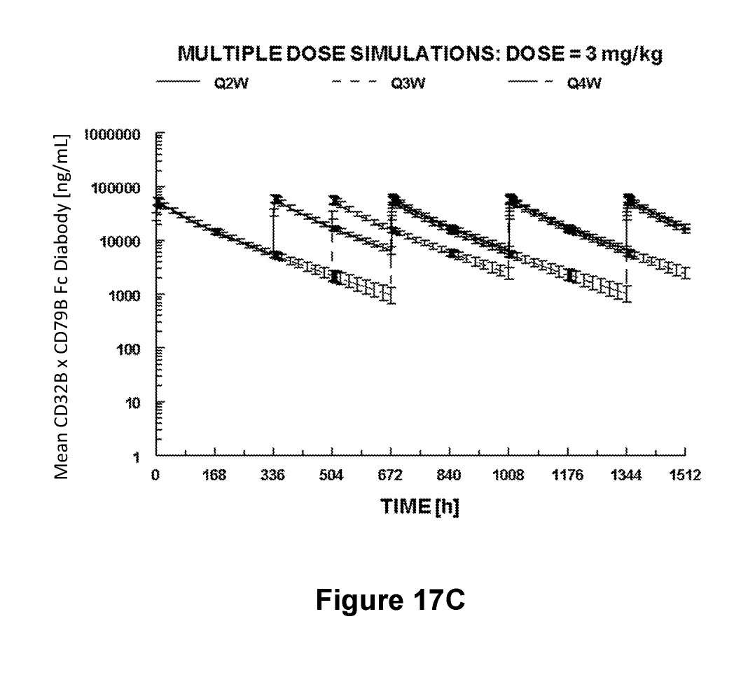

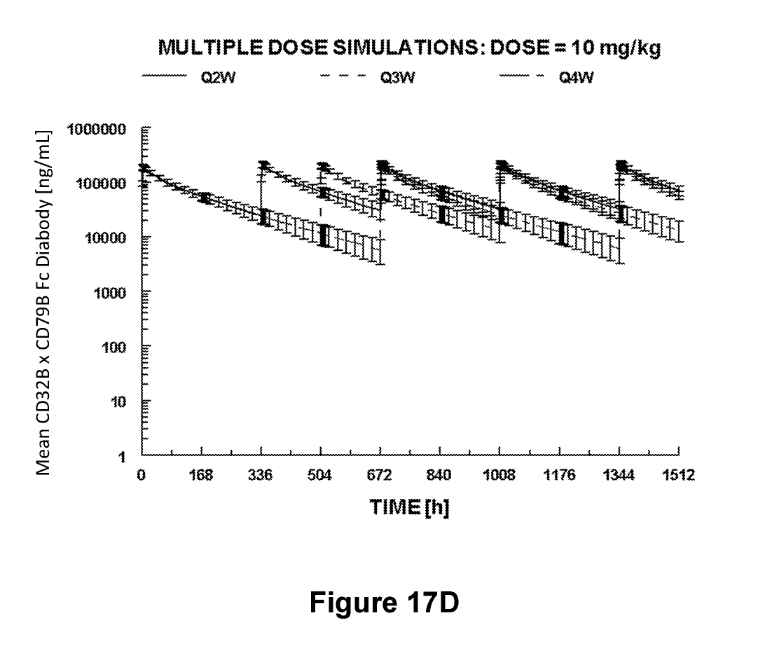

Individual subject pharmacokinetic data were modeled and best model parameter estimates were used to predict profiles for multiple dosing with administration in once per 2 week (Q2W), once per 3 week (Q3W) and once per 4 week (Q4W) dosing regimens. Doses investigated were 1, 3 and 10 mg/kg. Comparisons of exposure parameters (Cₘₐₓ, Cₘᵢₙ, and AUC) were performed and one- and two-compartment IV infusion models were investigated. The results of such modeling are shown for doses of 0.3 mg/kg subject body weight (FIG. 16A), 1 mg/kg subject body weight (FIG. 16B), 3 mg/kg subject body weight (FIG. 16C) and 10 mg/kg subject body weight (FIG. 16D) subject body weight for one dose per 2 week (Q2W), one dose per 3 week (Q3W) and one dose per 4 week (Q4W) dosing regimens. The predicted variability in the above-modeled profiles are presented (with SD) in FIGS. 17A-17D.

The Summary statistics for the exposure parameters after the first dose (Cₘₐₓ₁, Cₘᵢₙ₁, and AUC₁) and at steady-state (Cₘₐₓₛₛ, Cₘᵢₙₛₛ, and AUCₛₛ) are given in Tables 7, 8, and 9 for the 1, 3, and 10 mg/kg doses, respectively. All 3 doses were administered as Q2W, Q3W and Q4W regimens. In Tables 7, 8, 9 and 10 the AUC was determined over dosing intervals of 14, 21 and 28 days for the Q2W, Q3W and Q4W regimens, respectively.

TABLE 7
Simulated Exposure Parameters For 0.3 Mg/Kg Dose

| | | First Dose | | | Steady-State | | |
|---|---|---|---|---|---|---|---|
| | | Cₘₐₓ₁ | Cₘᵢₙ₁ | AUC₁ | Cₘₐₓₛₛ | Cₘᵢₙₛₛ | AUCₛₛ |
| | | (ng/ | (ng/ | (h*ng/ | (ng/ | (ng/ | (h*ng/ |
| Regimen | Attribute | mL) | mL) | mL) | mL) | mL) | mL) |
| 0.3 mg/kg | N | 6 | 6 | 6 | 6 | 6 | 6 |
| Q2W | GeoMean | 18468 | 399 | 1294521 | 18961 | 446 | 1359230 |
| | % CV | 21 | 45 | 14 | 20 | 51 | 14 |
| | Mean | 18793 | 429 | 1305069 | 19272 | 487 | 1369843 |
| | SD | 3718 | 162 | 182553 | 3698 | 208 | 189344 |
| | Median | 19759 | 475 | 1304448 | 20303 | 514 | 1358376 |
| | Min | 13706 | 219 | 1066584 | 14440 | 237 | 1127219 |
| | Max | 22482 | 603 | 1584875 | 23045 | 747 | 1683169 |
| 0.3 mg/kg | N | 6 | 6 | 6 | 6 | 6 | 6 |
| Q3W | GeoMean | 18468 | 107 | 1336474 | 18615 | 112 | 1359400 |
| | % CV | 21 | 92 | 14 | 21 | 98 | 14 |
| | Mean | 18793 | 134 | 1346858 | 18933 | 142 | 1370019 |
| | SD | 3718 | 85 | 184933 | 3698 | 95 | 189411 |
| | Median | 19759 | 128 | 1337058 | 19937 | 131 | 1358688 |
| | Min | 13706 | 33 | 1107607 | 13979 | 33 | 1127267 |
| | Max | 22482 | 252 | 1642310 | 22605 | 278 | 1683546 |
| 0.3 mg/kg | N | 6 | 6 | 6 | 6 | 6 | 6 |
| Q4W | GeoMean | 18468 | 31 | 1350494 | 18520 | 31 | 1359230 |
| | % CV | 21 | 182 | 14 | 21 | 187 | 14 |
| | Mean | 18793 | 49 | 1360943 | 18842 | 51 | 1369843 |
| | SD | 3718 | 43 | 186955 | 3706 | 45 | 189344 |
| | Median | 19759 | 36 | 1345334 | 19824 | 36 | 1358376 |
| | Min | 13706 | 4 | 1120902 | 13819 | 4 | 1127219 |
| | Max | 22482 | 114 | 1665734 | 22509 | 119 | 1683169 |

TABLE 8
Simulated Exposure Parameters For 1 Mg/Kg Dose

| | | First Dose | | | Steady-State | | |
|---|---|---|---|---|---|---|---|
| | | Cₘₐₓ₁ | Cₘᵢₙ₁ | AUC₁ | Cₘₐₓₛₛ | Cₘᵢₙₛₛ | AUCₛₛ |
| | | (ng/ | (ng/ | (h*ng/ | (ng/ | (ng/ | (h*ng/ |
| Regimen | Attribute | mL) | mL) | mL) | mL) | mL) | mL) |
| 1 mg/kg | N | 6 | 6 | 6 | 6 | 6 | 6 |
| Q2W | GeoMean | 22538 | 1099 | 1941942 | 23987 | 1292 | 2148025 |
| | % CV | 22 | 64 | 18 | 22 | 64 | 21 |
| | Mean | 23013 | 1236 | 1966948 | 24454 | 1467 | 2185411 |
| | SD | 5291 | 560 | 341430 | 5389 | 749 | 437841 |
| | Median | 21172 | 1307 | 1967100 | 23043 | 1503 | 2175560 |
| | Min | 17327 | 368 | 1547998 | 18798 | 461 | 1613971 |
| | Max | 31526 | 2079 | 2373060 | 33025 | 2744 | 2714620 |
| 1 mg/kg | N | 6 | 6 | 6 | 6 | 6 | 6 |
| Q3W | GeoMean | 22538 | 379 | 2066652 | 23044 | 413 | 2148987 |
| | % CV | 22 | 83 | 19 | 22 | 78 | 21 |
| | Mean | 23013 | 461 | 2098097 | 23504 | 501 | 2186425 |
| | SD | 5291 | 306 | 392549 | 5258 | 351 | 438369 |
| | Median | 21172 | 421 | 2098349 | 21884 | 434 | 2175963 |
| | Min | 17327 | 113 | 1582285 | 17862 | 143 | 1615276 |
| | Max | 31526 | 1019 | 2509219 | 31956 | 1160 | 2718422 |
| 1 mg/kg | N | 6 | 6 | 6 | 6 | 6 | 6 |
| Q4W | GeoMean | 22538 | 144 | 2114117 | 22732 | 150 | 2148025 |
| | % CV | 22 | 87 | 20 | 22 | 85 | 21 |
| | Mean | 23013 | 186 | 2148803 | 23197 | 195 | 2185411 |
| | SD | 5291 | 165 | 416875 | 5253 | 176 | 437841 |
| | Median | 21172 | 129 | 2147170 | 21465 | 130 | 2175560 |
| | Min | 17327 | 59 | 1595946 | 17534 | 69 | 1613971 |
| | Max | 31526 | 507 | 2599666 | 31654 | 538 | 2714620 |

TABLE 9
Simulated Exposure Parameters For 3 Mg/Kg Dose

| | | First Dose | | | Steady-State | | |
|---|---|---|---|---|---|---|---|
| | | Cₘₐₓ₁ | Cₘᵢₙ₁ | AUC₁ | Cₘₐₓₛₛ | Cₘᵢₙₛₛ | AUCₛₛ |
| | | (ng/ | (ng/ | (h*ng/ | (ng/ | (ng/ | (h*ng/ |
| Regimen | Attribute | mL) | mL) | mL) | mL) | mL) | mL) |
| 3 mg/kg | N | 6 | 6 | 6 | 6 | 6 | 6 |
| Q2W | GeoMean | 54486 | 5472 | 6488327 | 61116 | 6731 | 7566598 |
| | % CV | 18 | 13 | 10 | 18 | 16 | 10 |
| | Mean | 55202 | 5510 | 6515372 | 61868 | 6799 | 7599760 |
| | SD | 9342 | 706 | 649746 | 10185 | 1034 | 771685 |
| | Median | 57786 | 5467 | 6374280 | 64553 | 6923 | 7614517 |
| | Min | 40276 | 4847 | 5569784 | 46074 | 5383 | 6483798 |
| | Max | 64467 | 6320 | 7469875 | 71898 | 8065 | 8583898 |
| 3 mg/kg | N | 6 | 6 | 6 | 6 | 6 | 6 |
| Q3W | GeoMean | 54486 | 2216 | 7096298 | 56916 | 2461 | 7574231 |
| | % CV | 18 | 24 | 10 | 18 | 27 | 10 |
| | Mean | 55202 | 2268 | 7125716 | 57674 | 2529 | 7607408 |
| | SD | 9342 | 522 | 706806 | 9848 | 609 | 772412 |
| | Median | 57786 | 2292 | 7015300 | 60333 | 2618 | 7623535 |
| | Min | 40276 | 1529 | 6091270 | 42319 | 1579 | 6494141 |
| | Max | 64467 | 2950 | 8113876 | 67381 | 3282 | 8594913 |
| 3 mg/kg | N | 6 | 6 | 6 | 6 | 6 | 6 |
| Q4W | GeoMean | 54486 | 946 | 7353730 | 55451 | 1002 | 7566598 |
| | % CV | 18 | 40 | 10 | 18 | 41 | 10 |
| | Mean | 55202 | 1000 | 7384883 | 56193 | 1061 | 7599760 |
| | SD | 9342 | 328 | 738474 | 9608 | 346 | 771685 |
| | Median | 57786 | 1065 | 7330484 | 58842 | 1149 | 7614517 |
| | Min | 40276 | 482 | 6299207 | 41059 | 487 | 6483798 |
| | Max | 64467 | 1379 | 8365442 | 65674 | 1444 | 8583898 |

TABLE 10
Simulated Exposure Parameters For 10 Mg/Kg Dose

| | | First Dose | | | Steady-State | | |
|---|---|---|---|---|---|---|---|
| | | Cₘₐₓ₁ | Cₘᵢₙ₁ | AUC₁ | Cₘₐₓₛₛ | Cₘᵢₙₛₛ | AUCₛₛ |
| Regimen | Attribute | (ng/mL) | (ng/mL) | (h*ng/mL) | (ng/mL) | (ng/mL) | (h*ng/mL) |
| 10 mg/kg | N | 6 | 6 | 6 | 6 | 6 | 6 |
| Q2W | GeoMean | 187747 | 23684 | 22895439 | 218961 | 30796 | 28470994 |
| | % CV | 10 | 32 | 14 | 12 | 38 | 17 |
| | Mean | 188552 | 24638 | 23068595 | 220351 | 32493 | 28803009 |
| | SD | 19555 | 7243 | 3101087 | 27625 | 10968 | 4747240 |
| | Median | 184731 | 25253 | 23091722 | 216792 | 33549 | 28899625 |
| | Min | 167970 | 14286 | 19424051 | 184962 | 17285 | 22095139 |
| | Max | 223199 | 34575 | 27270524 | 268285 | 46008 | 35558762 |
| 10 mg/kg | N | 6 | 6 | 6 | 6 | 6 | 6 |
| Q3W | GeoMean | 187747 | 10983 | 25764809 | 200795 | 12486 | 28519275 |
| | % CV | 10 | 47 | 15 | 11 | 50 | 17 |
| | Mean | 188552 | 11863 | 25996110 | 201839 | 13614 | 28854051 |
| | SD | 19555 | 4719 | 3795674 | 22994 | 5715 | 4769110 |
| | Median | 184731 | 12454 | 25932750 | 198684 | 14095 | 28970836 |
| | Min | 167970 | 5649 | 20974439 | 174036 | 6172 | 22106333 |
| | Max | 223199 | 17340 | 31468974 | 242566 | 19802 | 35625556 |
| 10 mg/kg | N | 6 | 6 | 6 | 6 | 6 | 6 |
| Q4W | GeoMean | 187747 | 5283 | 27155195 | 193509 | 5621 | 28470994 |
| | % CV | 10 | 58 | 16 | 10 | 60 | 17 |
| | Mean | 188552 | 5907 | 27429401 | 194411 | 6320 | 28803009 |
| | SD | 19555 | 2789 | 4233452 | 21004 | 3058 | 4747240 |
| | Median | 184731 | 6087 | 27365849 | 191314 | 6443 | 28899625 |
| | Min | 167970 | 2423 | 21612851 | 170370 | 2520 | 22095139 |
| | Max | 223199 | 8864 | 33574511 | 231824 | 9756 | 35558762 |

As indicated in Tables 7, 8, 9 and 10 with regard to the first dose data, the Mean Cₘₐₓ₁ values were similar for the Q2W, Q3W, and Q4W regimens (i.e., for the 1 mg/kg dosing regimen, approximately 22 μg/mL; for the 3 mg/kg dosing regimen, approximately 54 μg/mL; and for the 10 mg/kg dosing regimen, approximately 187 μg/mL). Slight differences were observed in the mean AUC₁ values between the 3 regimens. The Mean Cmin1 (trough concentration) was observed to be higher for the Q2W regimen (i.e., for the 1 mg/kg dosing regimen, approximately 1.1 μg/mL; for the 3 mg/kg dosing regimen, approximately 5 μg/mL; and for the 10 mg/kg dosing regimen, approximately 24 μg/mL), which had a shorter dosing interval (14 days) compared to the Q4W regimen (i.e., for the 1 mg/kg dosing regimen, approximately 0.14 μg/mL; for the 3 mg/kg dosing regimen, approximately 0.95 μg/mL; and for the 10 mg/kg dosing regimen, approximately 5 μg/mL) which had a longer dosing interval of 28 days. The Mean Cmin1 for the Q3W regimen was observed to be intermediate to that of the Q2W and Q4W regimens (i.e., for the 1 mg/kg dosing regimen, approximately 0.38 μg/mL; for the 3 mg/kg dosing regimen, approximately 2.2 μg/mL; and for the 10 mg/kg dosing regimen, approximately 5 μg/mL).

As indicated in Tables 7, 8, 9 and 10 with regard to the Steady-State, the Mean Cₘₐₓₛₛ, Cₘᵢₙₛₛ, and AUCₛₛ were observed to be numerically higher compared to first dose values, and consistent with first dose data, the mean Cₘₐₓₛₛ of the Q2W, Q3W, and Q4W regimens were observed to be similar (<13% difference). Additionally, consistent with first dose data, slight differences were observed in the mean AUCₛₛ values between the 3 regimens. However, the dosing interval for the Q4W regimen is 28 days, and over the same interval, the number of doses administered for the Q2W regimen is 2-times higher than that of the Q4W regimen. Therefore, over the 28-day dosing interval, the AUCₛₛ for the Q2W regimen is expected to be approximately 2-times higher than that of the Q4W regimen. Regardless of the regimen administered, exposure parameters at steady-state suggest minimal accumulation of CD32B x CD79B Binding Molecule when compared to first dose data.

In sum, the Binding Molecules of the present invention have been administered to human subjects at dosages up to 10 mg/kg, and exhibited a clinical dose range of 0.3 mg/kg to 10 mg/kg. Higher dose rates may however, be additionally effective. For Q2W regimens, doses≥1 mg/kg should attain target concentration. For Q3W regimens, doses≥3 mg/kg should attain target concentration. Lower doses (e.g., 0.3 mg/kg) may however, be effective in achieving the desired biological activity. The administration was found to be safe in human healthy subjects and to demonstrate immunomodulatory activities. The administration of the Binding Molecules of the present invention was not followed by, or associated with, undesired cytokine release. Regardless of the dose administered, pharmacokinetic/pharmacodynamic (PK/PD) and modeling & simulation (M&S) analyses indicate that first dose or steady-state Cₘₐₓ values are similar for the Q2W, Q3W, and Q4W regimens. At steady-state, AUCₛₛ over a 28-day interval (dosing interval for the Q2W regimen) is expected to be approximately 2-times higher for the Q2W regimen compared to that of the Q4W regimen. First dose or steady-state Cₘᵢₙ values for the Q2W and Q3W regimens with shorter dosing intervals (14 and 21 days, respectively) are higher compared to Q4W regimen which has a longer dosing interval of 28 days (longer duration of washout).

Although the serum half-life (T_{1/2}) of the CD32B x CD79B Binding Molecules of the invention ranged from approximately 4-8 days, even after a first half-life had transpired, a significant proportion of administered CD32B x CD79B Binding Molecules were observed to remain bound to peripheral B-cells. For example, with respect to the CD32B x CD79B Fc Diabody of Example 1, greater than 20% occupancy was observed for at least 8 days at the 0.3 mg/kg dosage regimen, and for as long as 30 to almost 50 days at the 3 mg/kg and 10 mg/kg dosage regimens.

Inhibition of peripheral B-cell activation (particularly as reflected in AUC measurements) returns to baseline at about day 30. The CD32B x CD79B Binding Molecules also down-regulate the BCR target on memory B-cells (FIGS. 10A-10C) and on naïve B-cells (FIGS. 11A-11C) and this down-regulation also persists for about 27 days or longer. Similarly, the modulation of serum Ig levels (FIGS. 12A-12C) persists for at least 57 days at even the lowest dose. These results are from single dose administration. Accordingly, a preferred dosage schedule may be based on half-life, on the long acting biological activity of the CD32B x CD79B Binding Molecules, or on both half-life and on such long acting biological activity.

### Example 4

### Evaluation of the In Vivo Administration of CD32B x CD79B Bispecific Fc Diabodies on Humoral Immune Responses

As described herein, the CD32B x CD79B Binding Molecules, particularly the Bispecific Fc Diabodies, of the present invention are designed to inhibit activated B-cells by triggering a physiological negative feedback loop that is based on activation-inhibition coupling. The inhibitory effects of the CD32B x CD79B Binding Molecules on humoral immune responses in response to vaccination with an immunogen such as Keyhole Limpet Hemocyanin (KLH) may be investigated. In particular, based on extrapolation of PK data from non-human primates, when administered at 0.3 mg/kg circulating CD32B x CD79B Fc diabody will be maintained above a level to sustain 20% occupancy of CD32B x CD79B Fc diabody binding sites on peripheral blood B-cells for 6 days. Based on in vitro studies such occupancy levels would be predicted to sustain a minimum of 50% inhibition of BCR-mediated B-cell activation for at least 6 days. Considering immune responses to KLH vaccination in humans takes approximately 4 to 6 days, it is anticipated that implementation of KLH vaccination at in subjected administered CD32B x CD79B Binding Molecules at doses of ≥0.3 mg/ml will result in detectable pharmacodynamic activity as evidenced by inhibition of B-cell responses to vaccination with KLH antigen. Accordingly, KLH may be administered at a subcutaneous (SC) dose of 1.0 mg on Day 2 for all the subjects receiving a dose of CD32B x CD79B Binding Molecules of 0.3 mg/kg or higher. In addition, KLH vaccination may be given no less than 24 hours from infusion of the CD32B x CD79B Binding Molecule, in order to establish safety and tolerability of such CD32B x CD79B Binding Molecule for each subject prior to vaccination.

Anti-KLH IgG, and IgM titers and their percent change inhibition from baseline, and the proportion of subjects who mounted a quantifiable anti-KLH, IgG, and IgM responses postimmunization will be tabulated and summarized by dose panel and time. Differences in immune responses between CD32B x CD79B Binding Molecule treatment and placebo will be assessed using descriptive statistics. Additional analyses (e.g., exposure-response analysis) may be conducted if judged appropriate.

### Example 5

### Evaluation of the In Vivo Administration of CD32B x CD79B Bispecific Fc Diabodies on Humoral Immune Responses

As described above, the inhibitory effects of the CD32B x CD79B Binding Molecules on humoral immune responses may be evaluated in response to vaccination. Inactivated Hepatitis A vaccine (HAV) is an established neo-antigen used to assess immunization response in immune impaired conditions (Valdez H, et al. (2000) “Response To Immunization With Recall And Neoantigens After Prolonged Administration Of An Hiv-1 Protease Inhibitor-Containing Regimen,” ACTG 375 team. AIDS clinical trials group. AIDS 14:11-21) and among patients who use B-cell depleting therapies (Van Der Kolk L E, et al. (2002) “Rituximab Treatment Results In Impaired Secondary Humoral Immune Responsiveness,” Blood 100:2257-9. In this study, the inhibitory effects of the exemplary CD32B x CD79B Binding Molecule described above, are evaluated in response to HAV administration in normal healthy volunteers having a negative serologic Hepatitis A titer. Briefly, a single dose of CD32B x CD79B Binding Molecule or placebo is administered at 3 mg/kg or 10 mg/kg by IV infusion, subjects also receive a single dose of HAV (VAQTA® (Hepatitis A vaccine, inactivated, Merck, 50 U/1-mL) intramuscularly on Day 2. The impact of administration of the CD32B x CD79B Binding Molecule on immune responses to a single dose of HAV is evaluated by monitoring the appearance of serum IgG specific anti-HAV-specific antibody in the vaccinated subjects. ARCHITECT HAVAb-IgG assay (Abbott Laboratories) was used to detect the presence of HAV-specific IgG. The ARCHITECT HAVAb-IgG assay is a chemiluminescent microparticle immunoassay (CMIA) for the qualitative detection of IgG antibody to HAV in human serum or plasma. Quantitative assays were also employed with a modified Abbott system.

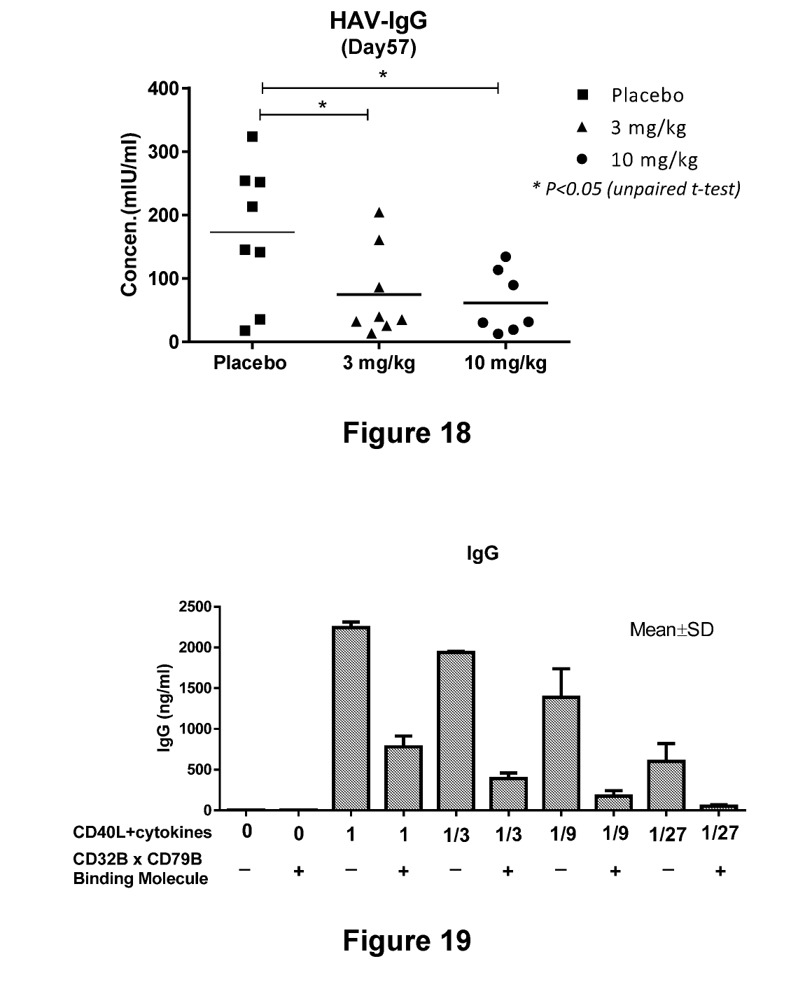

Table 11 summarizes the initial results from 10 subjects treated with placebo, or the exemplary CD32B x CD79B Binding Molecule at 3 mg/kg or 10 mg/kg. Table 12 summarizes the results at day 56 post-vaccination from all the subjects in the study. The mean concentration of HAV-specific IgG (anti-HAV IgG) present in the serum of each vaccinated group is summarized in Table 13. The concentration of anti-HAV IgG present in the serum of each of the vaccinated subjects is plotted in FIG. 18. The results of this study indicate that administration of CD32B x CD79B Binding Molecules inhibits activated B-cells and humoral immune responses in response to vaccination.

TABLE 11

| Group | Positive (n) | Negative (n) | Seroconversion Rate (%) |
|---|---|---|---|
| Placebo (n = 4) | 4 | 0 | 100 |
| 3 mg/kg (n = 3) | 1 | 2 | 33.3 |
| 10 mg/kg (n = 3) | 1 | 2 | 33.3 |

TABLE 12

| Group | Positive (n) | Negative (n) | Seroconversion Rate (%) |
|---|---|---|---|
| Placebo (n = 8) | 6 | 2 | 75 |
| 3 mg/kg (n = 8) | 3 | 5 | 37.5 |
| 10 mg/kg (n = 8) | 3 | 5 | 37.5 |

TABLE 13

HAV-IgG Concentration
(mean ± SC, mIU/mL)

| Group | Day 29 | Day 57 |
|---|---|---|
| Placebo | 34.3 ± 19.8 | 172.8 ± 108.3 |
| 3 mg/kg | 40.8 ± 1.9 | 74.5 ± 70.9 |
| 10 mg/kg | 16.2 ± 1.9 | 61.5 ± 49.7 |

### Example 6

### CD32B x CD79B Binding Molecules Block CD40 Dependent B-Cell Responses

As described above, administration of CD32B x CD79B binding molecules reduce the levels of the co-stimulatory molecule, CD40. The activity of the exemplary CD32B x CD79B Binding Molecule on CD40 dependent responses was evaluated in vitro. These studies use an assay system that mimics the process of B-cell differentiation into antibody (e.g., IgG) secreting cells in the presence of stimulation signals provided by follicular CD4-helper cells that occurs in the germinal center of secondary or territory lymphoid organs. Briefly, human B-cells were purified from peripheral whole blood of healthy donors using a negative selection kit. The purified human B-cells were cultured in complete RPMI1640 medium with or without stimulators (CD40-ligand (500 ng/mL), IL-4 (100 ng/mL) and IL-21 (20 ng/mL)), used undiluted, or as a 3-fold serial dilution (1, 1/3, 1/9, and 1/27), in the presence or absence of the exemplary CD32B x CD79B Binding Molecule described above (20 μg/mL) in a 5% CO₂ 37° C. incubator for 5 days. The culture supernatants were collected and the secreted human IgG was determined by ELISA. The results of this study are plotted in FIG. 19.

As shown in FIG. 19, CD32B x CD79B Binding Molecules can reduce IgG secretion, indicating that CD32B x CD79B Binding Molecules can inhibit CD40 mediated pathway related to IgG production.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method of treating an inflammatory disease or condition, or reducing or inhibiting an immune response, that comprises administering a therapeutically effective amount of a CD32B x CD79B Binding Molecule to a subject in need thereof, wherein said CD32B x CD79B Binding Molecule is capable of immunospecifically binding an epitope of CD32B and an epitope of CD79B, and wherein said CD32B x CD79B Binding Molecule is administered at a dose of between about 3 mg/kg and about 30 mg/kg, and at a dosage regimen of between one dose per week and one dose per 8 weeks.

2. (canceled)

3. The method of claim 1, wherein said CD32B x CD79B Binding Molecule is administered at a dose of about 3 mg/kg.

4. The method of claim 1, wherein said CD32B x CD79B Binding Molecule is administered at a dose of about 10 mg/kg.

5. The method of claim 1, wherein said CD32B x CD79B Binding Molecule is administered at a dose of about 30 mg/kg.

6. The method of claim 1, wherein said dosage regimen is one dose per 2 weeks (Q2W).

7. The method of claim 1, wherein said dosage regimen is one dose per 3 weeks (Q3W).

8. The method of claim 1, wherein said dosage regimen is one dose per 4 weeks (Q4W).

9. The method of claim 1, wherein said CD32B x CD79B Binding Molecule is a bispecific antibody that binds an epitope of CD32B and an epitope of CD79B, or a molecule that comprises the CD32B- and CD79B-binding domains of said bispecific antibody.

10. The method of claim 1, wherein said CD32B x CD79B Binding Molecule is a CD32B x CD79B bispecific diabody.

11. The method of claim 10, wherein said CD32B x CD79B bispecific diabody is a CD32B x CD79B Fc diabody.

12. The method of claim 1, wherein said inflammatory disease or condition is an autoimmune disease.

13. The method of claim 12, wherein said autoimmune disease is selected from the group consisting of: Addison's disease, autoimmune hepatitis, autoimmune inner ear disease myasthenia gravis, Crohn's disease, dermatomyositis, familial adenomatous polyposis, graft vs. host disease (GvHD), Graves' disease, Hashimoto's thyroiditis, lupus erythematosus, multiple sclerosis (MS); pernicious anemia, Reiter's syndrome, rheumatoid arthritis (RA), Sjogren's syndrome, systemic lupus erythematosus (SLE), type 1 diabetes, primary vasculitis, pemphigus, neuromyelitis optica, anti-NMDA receptor encephalitis, Guillain-Barré syndrome, chronic inflammatory demyelinating polyneuropathy (CIDP), Grave's opthalmopthy, IgG4 related diseases, idiopathic thrombocytopenic purpura (ITP), and ulcerative colitis.

14. The method of claim 13, wherein said inflammatory disease or condition is GvHD, RA, MS, or SLE.

15. The method of claim 1, wherein the serum level of an immunoglobulin is reduced by day 36 after administration of a first dose of said CD32B x CD79B Binding Molecule.

16. The method of claim 15, wherein said immunoglobulin is IgM, IgA or IgG.

17. The method of claim 1, wherein BCR-mediated peripheral B-cell activation is inhibited within 24 hours after administration of a first dose of said CD32B x CD79B Binding Molecule, wherein said B-cell activation is determined by an ex vivo calcium mobilization assay.

18. The method of claim 17, wherein said BCR-mediated B-cell activation is inhibited by at least 50%, and wherein said inhibition is sustained for at least 6 days.

19. The method of claim 1, wherein at least 20% of CD32B x CD79B binding sites on peripheral B-cell are occupied 6 hours after administration of a first dose of said CD32B x CD79B Binding Molecule.

20. The method of claim 1, wherein:
(A) the expression of CD40 on B-cells is down regulated; and/or
(B) CD40 mediated IgG secretion is inhibited.

21. The method of claim 1, wherein said subject is a human.

22. The method of claim 1, wherein said CD32B x CD79B Binding Molecule comprises:
(A) a VL_{CD32B} Domain that comprises the amino acid sequence of SEQ ID NO:30;
(B) a VH_{CD32B} Domain that comprises the amino acid sequence of SEQ ID NO:31;
(C) a VL_{CD79B} Domain that comprises the amino acid sequence of SEQ ID NO:32; and
(D) a VH_{CD79B} Domain that comprises the amino acid sequence of SEQ ID NO:33.

23. The method of claim 22, wherein said CD32B x CD79B Binding Molecule is an Fc diabody comprising:
(A) a first polypeptide chain that comprises the amino acid sequence of SEQ ID NO:39;
(B) a second polypeptide chain that comprises the amino acid sequence of SEQ ID NO:41; and
(C) a third polypeptide chain that comprises the amino acid sequence of SEQ ID NO:44.


SEQUENCE LISTING (48 sequences)

SEQ ID NO: 1 - protein, 107 aa, Homo sapiens [sequence omitted]

SEQ ID NO: 2 - protein, 104 aa, Homo sapiens
  MISC_FEATURE (1)..(104): Human IgG CL Lambda Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 3 - protein, 98 aa, Homo sapiens
  MISC_FEATURE (1)..(98): Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 4 - protein, 98 aa, Homo sapiens
  MISC_FEATURE (1)..(98): Human IgG2 CH1 Domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 5 - protein, 98 aa, Homo sapiens
  MISC_FEATURE (1)..(98): Human IgG4 CH1 Domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 6 - protein, 15 aa, Homo sapiens
  MISC_FEATURE (1)..(15): Human IgG1 Hinge Region
EPKSCDKTHTCPPCP

SEQ ID NO: 7 - protein, 12 aa, Homo sapiens
  MISC_FEATURE (1)..(12): Human IgG2 Hinge Region
ERKCCVECPPCP

SEQ ID NO: 8 - protein, 12 aa, Homo sapiens
  MISC_FEATURE (1)..(12): Human IgG4 Hinge Region
ESKYGPPCPSCP

SEQ ID NO: 9 - protein, 12 aa, Artificial Sequence
  Human IgG4 Hinge Region with S228P Substitution
ESKYGPPCPPCP

SEQ ID NO: 10 - protein, 217 aa, Homo sapiens
  MISC_FEATURE (1)..(217): CH2-CH3 Domain of Human IgG1
  MISC_FEATURE (217)..(217): XAA is Lysine (K) or is absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 11 - protein, 216 aa, Homo sapiens
  MISC_FEATURE (1)..(216): CH2-CH3 Domain of Human IgG2
  MISC_FEATURE (216)..(216): XAA is Lysine (K) or is absent
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 12 - protein, 217 aa, Homo sapiens
  MISC_FEATURE (1)..(217): CH2-CH3 Domain of Human IgG3
  MISC_FEATURE (217)..(217): XAA is Lysine (K) or is absent
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 13 - protein, 217 aa, Homo sapiens
  MISC_FEATURE (1)..(217): CH2-CH3 Domain of Human IgG4
  MISC_FEATURE (217)..(217): XAA is Lysine (K) or is absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 14 - protein, 8 aa, Artificial Sequence
  Preferred Intervening Spacer Peptide (Linker 1)
GGGSGGGG

SEQ ID NO: 15 - protein, 5 aa, Artificial Sequence
  Linker 2
ASTKG

SEQ ID NO: 16 - protein, 6 aa, Artificial Sequence
  Linker 2
GGCGGG

SEQ ID NO: 17 - protein, 7 aa, Artificial Sequence
  Heterodimer-Promoting Domain
GVEPKSC

SEQ ID NO: 18 - protein, 6 aa, Artificial Sequence
  Heterodimer-Promoting Domain
VEPKSC

SEQ ID NO: 19 - protein, 7 aa, Artificial Sequence
  Heterodimer-Promoting Domain
GFNRGEC

SEQ ID NO: 20 - protein, 6 aa, Artificial Sequence
  Heterodimer-Promoting Domain
FNRGEC

SEQ ID NO: 21 - protein, 28 aa, Artificial Sequence
  “E-coil” Heterodimer-Promoting Domain
EVAALEKEVAALEKEVAALEKEVAALEK

SEQ ID NO: 22 - protein, 28 aa, Artificial Sequence
  “K-coil” Heterodimer-Promoting Domain
KVAALKEKVAALKEKVAALKEKVAALKE

SEQ ID NO: 23 - protein, 28 aa, Artificial Sequence
  Cysteine-Containing “E-Coil” Heterodimer- Promoting Domain
EVAACEKEVAALEKEVAALEKEVAALEK

SEQ ID NO: 24 - protein, 28 aa, Artificial Sequence
  Cysteine-Containing “K-Coil” Heterodimer- Promoting Domain
KVAACKEKVAALKEKVAALKEKVAALKE

SEQ ID NO: 25 - protein, 10 aa, Artificial Sequence
  Cysteine-Containing Peptide (Peptide 1)
DKTHTCPPCP

SEQ ID NO: 26 - protein, 13 aa, Artificial Sequence
  Cysteine-Containing Peptide (Peptide 1)
GGGDKTHTCPPCP

SEQ ID NO: 27 - protein, 5 aa, Artificial Sequence
  Intervening Linker Peptide (Linker 3)
APSSS

SEQ ID NO: 28 - protein, 8 aa, Artificial Sequence
  Intervening Linker Peptide (Linker 3)
APSSSPME

SEQ ID NO: 29 - protein, 5 aa, Artificial Sequence
  Spacer Peptide (Linker 4)
GGGNS

SEQ ID NO: 30 - protein, 107 aa, Mus musculus
  MISC_FEATURE (1)..(107): VL Domain of Anti-Human CD32B Antibody
DIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIK

SEQ ID NO: 31 - protein, 116 aa, Mus musculus
  MISC_FEATURE (1)..(116): VH Domain of Anti-Human CD32B Antibody
EVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSS

SEQ ID NO: 32 - protein, 112 aa, Mus musculus
  MISC_FEATURE (1)..(112): VL Domain of Anti-Human CD79B Antibody
DVVMTQSPLSLPVTLGQPASISCKSSQSLLDSDGKTYLNWFQQRPGQSPNRLIYLVSKLDSGVPDRFSGSGSGTDFTLKISRVEAEDVGVYYCWQGTHFPLTFGGGTKLEIK

SEQ ID NO: 33 - protein, 113 aa, Mus musculus
  MISC_FEATURE (1)..(113): VH Domain of Anti-Human CD79B Antibody
QVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSS

SEQ ID NO: 34 - protein, 262 aa, Artificial Sequence
  First Polypeptide Chain of First Exemplary CD32B x CD79B Bispecific Diabody
DIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIKGGGSGGGGQVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSSGGCGGGEVAALEKEVAALEKEVAALEKEVAALEK

SEQ ID NO: 35 - protein, 270 aa, Artificial Sequence
  Second Polypeptide Chain of First Exemplary CD32B x CD79B Bispecific Diabody
DVVMTQSPLSLPVTLGQPASISCKSSQSLLDSDGKTYLNWFQQRPGQSPNRLIYLVSKLDSGVPDRFSGSGSGTDFTLKISRVEAEDVGVYYCWQGTHFPLTFGGGTKLEIKGGGSGGGGEVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSSGGCGGGKVAALKEKVAALKEKVAALKEKVAALKE

SEQ ID NO: 36 - protein, 261 aa, Artificial Sequence
  First Polypeptide Chain of Second Exemplary CD32B x CD79B Bispecific Diabody
DIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIKGGGSGGGGQVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSSASTKGEVAACEKEVAALEKEVAALEKEVAALEK

SEQ ID NO: 37 - protein, 269 aa, Artificial Sequence
  Second Polypeptide Chain of Second Exemplary CD32B x CD79B Bispecific Diabody
DVVMTQSPLSLPVTLGQPASISCKSSQSLLDSDGKTYLNWFQQRPGQSPNRLIYLVSKLDSGVPDRFSGSGSGTDFTLKISRVEAEDVGVYYCWQGTHFPLTFGGGTKLEIKGGGSGGGGEVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSSASTKGKVAACKEKVAALKEKVAALKEKVAALKE

SEQ ID NO: 38 - protein, 217 aa, Artificial Sequence
  CH2-CH3 Domain of Knob-Bearing IgG Fc Region
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 502 aa, Artificial Sequence
  First Polypeptide Chain of First Exemplary CD32B x CD79B Bispecific Fc Diabody
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAPSSSPMEDIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIKGGGSGGGGQVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSSGGCGGGEVAALEKEVAALEKEVAALEKEVAALEKGGGNS

SEQ ID NO: 40 - DNA, 1506 bp, Artificial Sequence
  Polynucleotide Encoding First Polypeptide Chain of First Exemplary CD32B x CD79B Bispecific Fc Diabody
gacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagccccttccagctcccctatggaagacatccagatgacccagtctccatcctccttatctgcctctgtgggagatagagtcaccatcacttgtcgggcaagtcaggaaattagtggttacttaagctggctgcagcagaaaccaggcaaggcccctagacgcctgatctacgccgcatccactttagattctggtgtcccatccaggttcagtggcagtgagtctgggaccgagttcaccctcaccatcagcagccttcagcctgaagattttgcaacctattactgtctacaatattttagttatccgctcacgttcggaggggggaccaaggtggaaataaaaggaggcggatccggcggcggaggccaggttcagctggtgcagtctggagctgaggtgaagaagcctggcgcctcagtgaaggtctcctgcaaggcttctggttacacctttaccagctactggatgaactgggtgcgacaggcccctggacaagggcttgagtggatcggaatgattgatccttcagacagtgaaactcactacaatcaaaagttcaaggacagagtcaccatgaccacagacacatccacgagcacagcctacatggagctgaggagcctgagatctgacgacacggccgtgtattactgtgcgagagctatgggctactgggggcaagggaccacggtcaccgtctcctccggaggatgtggcggtggagaagtggccgcactggagaaagaggttgctgctttggagaaggaggtcgctgcacttgaaaaggaggtcgcagccctggagaaaggcggcgggaactct

SEQ ID NO: 41 - protein, 270 aa, Artificial Sequence; identical to SEQ ID NO: 35
  Second Polypeptide Chain of First Exemplary CD32B x CD79B Bispecific Fc Diabody

SEQ ID NO: 42 - DNA, 810 bp, Artificial Sequence
  Polynucleotide Encoding Second Polypeptide Chain of First Exemplary CD32B x CD79B Bispecific Fc Diabody
gatgttgtgatgactcagtctccactctccctgcccgtcacccttggacagccggcctccatctcctgcaagtcaagtcagagcctcttagatagtgatggaaagacatatttgaattggtttcagcagaggccaggccaatctccaaaccgcctaatttatctggtgtctaaactggactctggggtcccagacagattcagcggcagtgggtcaggcactgatttcacactgaaaatcagcagggtggaggctgaggatgttggggtttattactgctggcaaggtacacattttccgctcacgttcggcggagggaccaagcttgagatcaaaggaggcggatccggcggcggaggcgaagtgcagcttgtggagtctggaggaggcttggtgcaacctggaggatccctgagactctcttgtgccgcctctggattcacttttagtgacgcctggatggactgggtccgtcaggccccaggcaaggggcttgagtgggttgctgaaattagaaacaaagctaaaaatcatgcaacatactatgctgagtctgtgatagggaggttcaccatctcaagagatgacgccaaaaacagtctgtacctgcaaatgaacagcttaagagctgaagacactgccgtgtattactgtggggctctgggccttgactactggggccaaggcaccctggtgaccgtctcctccggaggatgtggcggtggaaaagtggccgcactgaaggagaaagttgctgctttgaaagagaaggtcgccgcacttaaggaaaaggtcgcagccctgaaagag

SEQ ID NO: 43 - protein, 217 aa, Artificial Sequence
  CH2-CH3 Domain of Hole-Containing IgG Fc Region
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 44 - protein, 227 aa, Artificial Sequence
  Third Polypeptide Chain of First Exemplary CD32B x CD79B Bispecific Fc Diabody
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 45 - DNA, 681 bp, Artificial Sequence
  Polynucleotide Encoding Third Polypeptide Chain of First Exemplary CD32B x CD79B Bispecific Fc Diabody
gacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagttgcgcagtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccgctacacgcagaagagcctctccctgtctccgggtaaa

SEQ ID NO: 46 - protein, 501 aa, Artificial Sequence
  First Polypeptide Chain of Second Exemplary CD32B x CD79B Bispecific Fc Diabody
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAPSSSPMEDIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIKGGGSGGGGQVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSSASTKGEVAACEKEVAALEKEVAALEKEVAALEKGGGNS

SEQ ID NO: 47 - protein, 269 aa, Artificial Sequence; identical to SEQ ID NO: 37
  Second Polypeptide Chain of Second Exemplary CD32B x CD79B Bispecific Fc Diabody

SEQ ID NO: 48 - protein, 227 aa, Artificial Sequence; identical to SEQ ID NO: 44
  Third Polypeptide Chain of Second Exemplary CD32B x CD79B Bispecific Fc Diabody